(19) United States
(12) Patent Application Publication
(10) Pub. No.: US 2026/0308108 A1
(43) Pub. Date: Oct. 8, 2026

(54) COMPOSITIONS AND METHODS FOR THE TREATMENT OF OCULAR DISORDERS

(71) Applicant: Case Western Reserve University, Cleveland, OH (US)

(72) Inventors: Gregory Tochtrop, Irvine, CA (US); Philip Kiser, Irvine, CA (US); Marco Bassetto, Irvine, CA (US); Mohsen Badiee, Cleveland, OH (US)

(21) Appl. No.: 19/482,865

(22) PCT Filed: May 8, 2024
(86) PCT No.: PCT/US2024/028253

§ 371 Date: Nov. 10, 2025

Related U.S. Application Data
(60) Provisional application No. 63/500,819, filed on May 8, 2023.

(51) Int. Cl. C07C 217/72; A61K 31/137; A61P 27/02; C07B 59/00

(52) U.S. Cl. CPC C07C 217/72; A61K 31/137; A61P 27/02; C07B 59/001; C07B 2200/05; C07C 2601/14

(57) ABSTRACT

A method of treating an ocular disorder, such as light induced retinal degeneration, macular degeneration, Stargardt's disease, geographic atrophy, and retinitis pigmentosa, in a subject in need thereof, includes administering to the subject a therapeutically effective amount of a compound of formula (I), a 3-(cyclohexylmethoxy)-benzaldehyde moiety connected to a gamma-hydroxyamine moiety, which may be fluoro-substituted, as a retinoid isomerase (RPE65) inhibitor, or a pharmaceutically acceptable salt, tautomer, or solvate thereof.

### RELATED APPLICATION

This application claims priority from U.S. Provisional Application No. 63/500,819, filed May 8, 2023, the subject matter of which is incorporated herein by reference in its entirety.

### GOVERNMENT FUNDING

This invention was made with government support under 1904530 awarded by the National Science Foundation, and 101BX004939 awarded by the Department of Veterans Administration. The government has certain rights in the invention.

### BACKGROUND

Retinal photoreceptor cells can respond to light throughout life because they continuously regenerate a light-sensitive chromophore and photoreceptor structures. Defects in various proteins involved in these processes cause photoreceptor degeneration. Light-detection is mediated by a group of G protein-coupled receptor proteins called opsins located in rod and cone photoreceptor cells of the retina. The light-absorbing chromophore of most vertebrate opsins is 11-cis-retinal. Absorption of a photon by an opsin pigment causes isomerization of the chromophore to all-trans-retinal. Regeneration of the visual chromophore following light exposure is dependent upon an enzymatic pathway referred to as the visual cycle.

To understand why human vision declines with age, considerable research has focused on the retina, especially the layer of rod and cone photoreceptor cells that convert light into electrical signals. However, age-related decreases in retinal photoreceptor cell function cannot be explained alone by rod/cone cell loss, abnormal retinal plasticity, or any acute signs of retinal disease. Rather, there are pathological events that take place over time, including the aberrant metabolism of all-trans-retinal that can interfere with normal photoreceptor function.

All-trans-retinal, when released from rhodopsin, primarily re-enters the visual cycle. However, in some individuals, it also can persist as an unbound potentially toxic aldehyde or react with other molecules to create toxic compounds such as N-retinylidene-N-retinylethanolamine (A2E) and retinal dimers. These events are thought to contribute to the etiology of blinding diseases such as age-related macular degeneration (AMD) and Stargardt disease.

Various therapeutic strategies have been developed to combat the aberrant metabolism of retinoids including all-trans-retinal. One of these involves the inhibition of the retinoid isomerase (RPE65), a key enzyme of the visual cycle, to slow the generating of all-trans-retinal without significantly impairing vision. Complete suppression of RPE65 activity, however, has undesirable blinding consequences, thus only attenuated or partial suppression of RPE65 is desirable. A second strategy is to lower the toxic effects of unbound all-trans-retinal and temporarily sequester it by adduction to a primary amine. Since most RPE65 inhibitors contain an amino group, they can play a dual role to both slow the metabolism of all-trans-retinal as well as sequester it.

### SUMMARY

Embodiments described herein relate to compounds for use in the treatment of ocular disorders, such as ocular diseases and disorders related to aberrant all-trans-retinal accumulation in a subject's ocular tissue. It has been discovered that all-trans-retinal, a retinoid metabolite naturally produced during visual processing, is highly toxic when present at elevated levels. To lower its toxicity, therapeutic compounds have been identified below that can be delivered to and retained in the eye to modulate the visual (retinoid) cycle.

In some embodiments, the compounds described herein have strategically incorporated deuterium and/or fluorine to modulate the compounds' potency and metabolism. Regioselective incorporation of fluorine can impact pKa modulation; alter target selectivity through conformational variations or changes in specific hydrophobic interactions; and alter tissue-specific penetration (e.g., central nervous system (CNS)), through modification of lipophilicity. These effects of fluorination are in addition to the well-established strategy of replacing metabolically labile hydrogens with C—F bonds. Regioselective incorporation of deuterium can be used to attenuate amine oxidation and rapid metabolic elimination via engineering a localized primary isotope effect. Collectively, the compounds have improved potency, absorption, selectivity, and metabolism to mitigate the toxicity of all-trans-retinal in age-related blindness.

Other embodiments described herein relate to a compound that has formula (I):

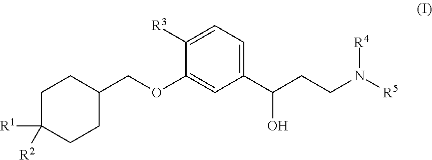

or a pharmaceutically acceptable salt, tautomer, or solvate thereof, wherein:
R¹, R², and R³ are each independently H or F;
R⁴ and R⁵ are each independently H, D, or an alkyl, which is optionally substituted with one or more D or F, and wherein at least one of R⁴ or R⁵ is an alkyl, which is optionally substituted with one or more D or F.

In some embodiments, at least one of R¹, R², or R³ is F or at least one of R⁴ or R⁵ is an alkyl substituted with one or more D or F.

In some embodiments, at least one of R¹ or R² is F.

In other embodiments, both of R¹ and R² are F.

In some embodiments, R³ is F.

In some embodiments, at least one R⁴ or R⁵ is an alkyl substituted with one or more D or F.

In other embodiments, at least one R⁴ or R⁵ is C₁-C₆ alkyl substituted with one or more D or F.

In other embodiments, both of R⁴ and R⁵ are C₁-C₆ alkyl substituted with one or more D or F.

In some embodiments, at least one R⁴ or R⁵ is methyl substituted with one or more D or F.

In other embodiments, both of R⁴ and R⁵ are methyl substituted with one or more D or F.

In still other embodiments, at least one R⁴ or R⁵ is —CD₃ or —CF₃.

In yet other embodiments, both of R⁴ and R⁵ are —CD₃ or —CF₃.

Other embodiments relate to a compound selected from:

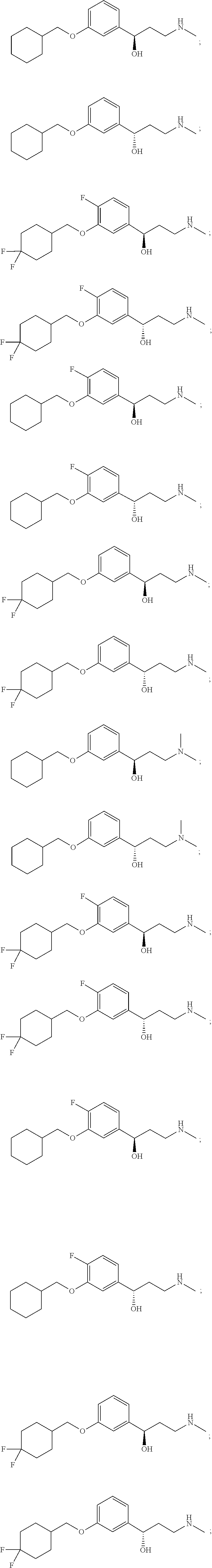

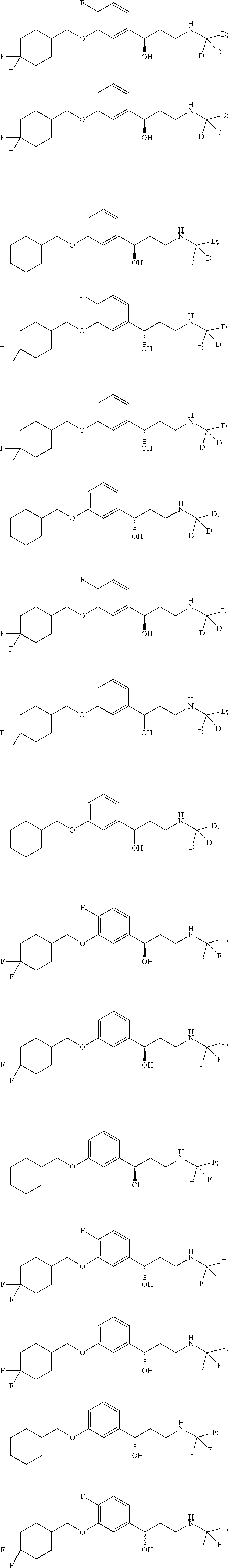

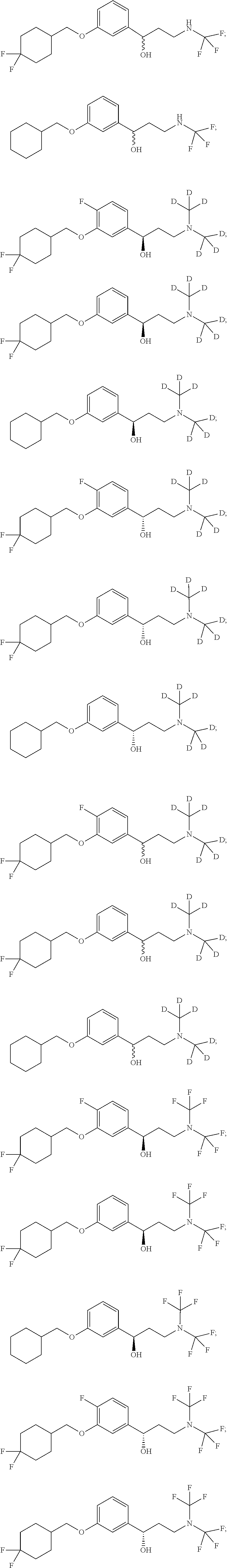

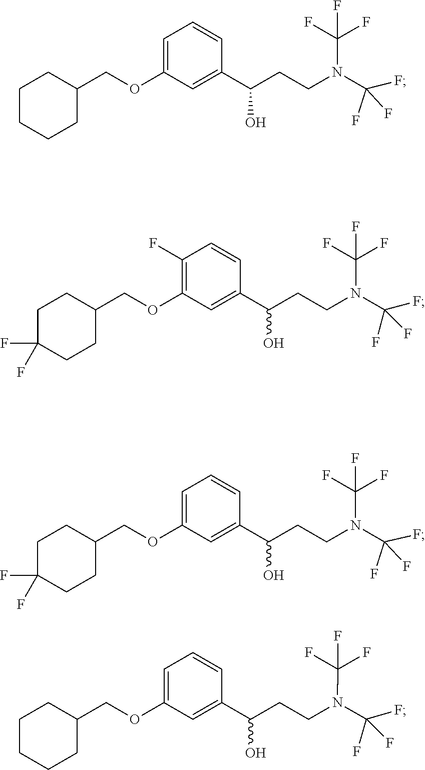 [SMILES: O[C@@H](CCN(C(F)(F)F)C(F)(F)F)c1cccc(OCC2CCCCC2)c1 ; OC(CCN(C(F)(F)F)C(F)(F)F)c1ccc(F)c(OCC2CCC(F)(F)CC2)c1 ; OC(CCN(C(F)(F)F)C(F)(F)F)c1cccc(OCC2CCC(F)(F)CC2)c1 ; OC(CCN(C(F)(F)F)C(F)(F)F)c1cccc(OCC2CCCCC2)c1]

or a pharmaceutically acceptable salt, tautomer, or solvate thereof.

Still other embodiments relate to a pharmaceutical composition comprising a compound described herein and a pharmaceutically acceptable carrier.

Other embodiments relate to a method of treating an ocular disorder in a subject in need thereof. The method includes administering to the subject a therapeutically effective amount of a compound as described herein or a pharmaceutical composition comprising the compound described herein.

In some embodiments, the pharmacokinetics of the compound upon administration to the subject is such that the compound does not promote night blindness when administered daily.

In other embodiments, the compound is not deaminated by VAP-1 enzyme.

In some embodiments, the compound can inhibit RPE65 enzymatic activity in the subject.

In some embodiments, the compound can be administered by enteral administration.

In some embodiments, the ocular disorder can include at least one of light induced retinal degeneration, macular degeneration, Stargardt's disease, geographic atrophy, and retinitis pigmentosa.

### BRIEF DESCRIPTION OF THE DRAWINGS

FIGS. 1(A-D) illustrate inhibition of visual cycle using emixustat-like inhibitors. (A) Emixustat inhibits a crucial step of the visual cycle by blocking the isomerization of all-trans-RE to 11-cis-ROL catalyzed by RPE65. Abbreviations: hv, photon; 11cROL, 11-cis-retinol; 11cRAL, 11-cis-retinal; RAL, all-trans-retinal; ROL, all-trans-retinol; RE, retinyl esters; RPE65, retinal pigment epithelium protein 65 kDa. (B) Chemical structure of targeted RPE65 inhibitors. (C) Two-dimensional interaction diagram for emixustat and palmitate ligands in RPE65's active site. Polar interactions are represented with dashed lines. Residues involved in nondipolar interactions are shown as spiked arcs. (D) Modifications of emixustat's scaffold described here: (i) γ-amino-α-propanol geometric derivatives (parent molecule is in the meta configuration); (ii) methylated derivatives; (iii) α/β-amino-γ-propanol derivatives; (iv) truncated analogues.

FIGS. 2(A-C) illustrate plots showing in vitro inhibition of RPE65 isomerase by synthesized compounds. Chemical structures and corresponding dose-response curves of (A) γ-amino-α-aryl alcohols 3a-c, (B) ethylamines 11a-d, and (C) propylamines 16a-c and 16e. All data points are plotted as mean t SD representing 3 replicates per point.

FIGS. 3(A-D) illustrate inhibition of RPE65 in vivo. (A) Schematic showing the timeline of the experiment. IP, intraperitoneal injection. (B) Representative HPLC chromatograms obtained from retinoid extracts of dark-adapted mice (black trace), and mice treated with vehicle (DMSO, green line), 3a (blue trace), and 16e (purple trace) that were dark-adapted for four (left) and 24 h (right) after exposure to light. The two solid lines indicate the peaks corresponding to the RE (a) and the 11cRAL oxime (syn) (b). Insets show the spectral identity of 11cRAL oxime (syn) (a) and the RE (b), respectively. (C, D) Kinetics of 11cRAL synthesis and RE clearance after IP injection of vehicle or test compounds for RPE65 inhibition. The dashed line indicates mean baseline levels of 11cRAL and RE from dark-adapted mice. Mean values t SD, n=3, and individual data points are shown in the graph.

FIGS. 4(A-E) illustrate test of VAP-1-dependent oxidative metabolism of compound 16e. (A) Scheme showing the oxidation of 3a (left) and 16e (right) by VAP-1 present in the aorta homogenates used for the assay. (B, C) Representative HPLC chromatograms obtained from homogenates of mice aortas showing the time course of oxidative metabolism by VAP-1 on 3a (blue traces) and 16e (purple traces). (D) Comparison between the UV/vis absorbance spectra of the peaks labeled in (B) and (C) and the UV/vis absorbance spectra of the synthetic standard ACU-5201. (E) Kinetics of product formation for 3a (blue columns) and 16e (purple columns). Mean values t SD, n=3 replicates, and individual data points are shown in the graph.

FIGS. 5(A-G) illustrate evaluation of 16e hepatic metabolism. (A) Scheme illustrating the possible synthesis of 3a from 16e by liver microsomes. (B) Representative HPLC chromatograms obtained from liver microsomes showing the time course of 3a (blue traces) and 16e (purple traces) metabolism. 3a and 16e were used at a concentration of 200 μM. (C) Plot showing the relationship between V₀ and concentration of 3a (blue trace) and 16e (purple trace). Single data are plotted and fitted with Michaelis-Menten equation using GraphPad software. 90% confidence intervals are shown as dotted liens. Progress of 16e elimination in the mouse eye (D) and serum (E). Mean values±SD, n=3 replicates, and individual data points are shown in the graph. Quantification of 16e and 3a in the mouse eye (F) and serum (G) 1 h after receiving an intraperitoneal injection of 3a or vehicle (DMSO), mean values±SD, n=3 replicates and individual data points are shown in the graph.

FIGS. 6(A-B) illustrate crystal structure of RPE65 in complex with 16e. (A) Cut-through view of the RPE65 active site showing the binding location of 16e as well as a coordinated palmitate ligand. The corresponding 2F₀-F_{c} electron density map, contoured at 1 RMSD, is shown as blue mesh within 2 Å of the bound ligands. (B) Illustration of RPE65 residues within 4.5 Å from the bound ligands. Thr147 and Glu148, which are suggested to be important for inhibitor binding, are shown as gray sticks. The PDB accession code for the structure 8DOC.

### DETAILED DESCRIPTION

For convenience, certain terms employed in the specification, examples, and appended claims are collected here. Unless defined otherwise, all technical and scientific terms used herein have the same meaning as commonly understood by one of ordinary skill in the art to which this application belongs.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.,”, as used herein are non-limiting and are for illustrative purposes only. “Including” and “including but not limited to” are used interchangeably.

The term “or” as used herein should be understood to mean “and/or”, unless the context clearly indicates otherwise.

It will be noted that the structure of some of the compounds of the application include asymmetric (chiral) carbon atoms. It is to be understood accordingly that the isomers arising from such asymmetry are included within the scope of the invention, unless indicated otherwise. Such isomers can be obtained in substantially pure form by classical separation techniques and by stereochemically controlled synthesis. The compounds of this application may exist in stereoisomeric form, therefore, can be produced as individual stereoisomers or as mixtures.

The term “isomerism” refers to compounds that have identical molecular formulae but that differ in the nature or the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images are termed “enantiomers”, or sometimes optical isomers. A carbon atom bonded to four nonidentical substituents is termed a “chiral center”.

The term “chiral isomer” refers to a compound with at least one chiral center. It has two enantiomeric forms of opposite chirality and may exist either as an individual enantiomer or as a mixture of enantiomers. A mixture containing equal amounts of individual enantiomeric forms of opposite chirality is termed a “racemic mixture”. A compound that has more than one chiral center has 2n-1 enantiomeric pairs, where n is the number of chiral centers. Compounds with more than one chiral center may exist as either an individual diastereomer or as a mixture of diastereomers, termed a “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al. Angew. Chem. Inter. Edit. 1966, 5, 385; errata 511; Cahn et al., Angew. Chem. 1966, 78, 413; Cahn and Ingold, J Chem. Soc. 1951 (London), 612; Cahn et al., Experientia 1956, 12, 81; Cahn, J., Chem. Educ. 1964, 41, 116).

The term “geometric isomers” refer to the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Further, the structures and other compounds discussed in this application include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

The terms “crystal polymorphs” or “polymorphs” or “crystal forms” means crystal structures in which a compound (or salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

The term “derivative”, refers to compounds that have a common core structure, and are substituted with various groups as described herein. For example, all of the compounds represented by formula I are secondary or tertiary amines and have formula I as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physiochemically or topologically based. Examples of carboxylic acid bioisosteres include acyl sulfonimides, tetrazoles, sulfonates, and phosphonates. See, e.g., Patani and LaVoie, Chem. Rev. 96, 3147-3176 (1996).

The phrases “parenteral administration” and “administered parenterally” refer to modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The term “treating” refers to inhibiting a disease, disorder or condition in a subject, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “preventing” refers to stopping a disease, disorder or condition from occurring in a subject, which may be predisposed to the disease, disorder and/or condition but has not yet been diagnosed as having it. Preventing a condition related to a disease includes stopping the condition from occurring after the disease has been diagnosed but before the condition has been diagnosed.

The term a “pharmaceutical composition” refers to a formulation containing the disclosed compounds in a form suitable for administration to a subject. The pharmaceutical composition can be in bulk or in unit dosage form. The unit dosage form is any of a variety of forms, including, for example, a capsule, an IV bag, a tablet, a single pump on an aerosol inhaler, or a vial. The quantity of active ingredient (e.g., a formulation of the disclosed compound or salts thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including ocular, oral, pulmonary, rectal, parenteral, transdermal, subcutaneous, intravenous, intramuscular, intraperitoneal, intranasal, and the like. Dosage forms for the topical or transdermal administration of a compound of this invention include powders, sprays, ointments, pastes, creams, lotions, gels, solutions, patches and inhalants. In a preferred embodiment, the active compound is mixed under sterile conditions with a pharmaceutically acceptable carrier, and with any preservatives, buffers, or propellants that are required.

The term “flash dose” refers to compound formulations that are rapidly dispersing dosage forms.

The term “immediate release” refers to a release of compound from a dosage form in a relatively brief period of time, generally up to about 60 minutes. The term “modified release” is defined to include delayed release, extended release, and pulsed release. The term “pulsed release” is defined as a series of releases of drug from a dosage form. The term “sustained release” or “extended release” is defined as continuous release of a compound from a dosage form over a prolonged period.

The phrase “pharmaceutically acceptable” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” refers to pharmaceutically acceptable materials, compositions or vehicles, such as a liquid or solid filler, diluent, excipient, solvent or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar, (14) buffering agents, such as magnesium hydroxide and aluminum hydroxide; (15) alginic acid; (16) pyrogen-free water, (17) isotonic saline; (18) Ringer's solution; (19) ethyl alcohol; (20) phosphate buffer solutions; and (21) other non-toxic compatible substances employed in pharmaceutical formulations.

The compounds of the application are capable of further forming salts. All of these forms are also contemplated within the scope of the claims.

The phrase “pharmaceutically acceptable salt” of a compound means a salt that is pharmaceutically acceptable and that possesses the desired pharmacological activity of the parent compound. For example, the salt can be an acid addition salt. One embodiment of an acid addition salt is a hydrochloride salt.

The pharmaceutically acceptable salts can be synthesized from a parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of salts are found in Remington's Pharmaceutical Sciences, 18th ed. (Mack Publishing Company, 1990). For example, salts can include, but are not limited to, the hydrochloride and acetate salts of the aliphatic amine-containing, hydroxyl amine-containing, and imine-containing compounds of the present invention.

It should be understood that all references to pharmaceutically acceptable salts include solvent addition forms (solvates) or crystal forms (polymorphs) as defined herein, of the same salt.

The compounds described herein can also be prepared as esters, for example pharmaceutically acceptable esters. For example, a carboxylic acid function group in a compound can be converted to its corresponding ester, e.g., a methyl, ethyl, or other ester. Also, an alcohol group in a compound can be converted to its corresponding ester, e.g., an acetate, propionate, or other ester.

The compounds described herein can also be prepared as prodrugs, for example pharmaceutically acceptable prodrugs. The terms “pro-drug” and “prodrug” are used interchangeably herein and refer to any compound, which releases an active parent drug in vivo. Since prodrugs are known to enhance numerous desirable qualities of pharmaceuticals (e.g., solubility, bioavailability, manufacturing, etc.) the compounds of the present invention can be delivered in prodrug form. Thus, the present application is intended to cover prodrugs of the presently claimed compounds, methods of delivering the same and compositions containing the same. “Prodrugs” are intended to include any covalently bonded carriers that release an active parent drug in vivo when such prodrug is administered to a subject. Prodrugs the present invention are prepared by modifying functional groups present in the compound in such a way that the modifications are cleaved, either in routine manipulation or in vivo, to the parent compound. Prodrugs include compounds described herein wherein a hydroxy, amino, sulfhydryl, carboxy, or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates, and benzoate derivatives) and carbamates (e.g., N,N-dimethylaminocarbonyl) of hydroxy functional groups, ester groups (e.g., ethyl esters, morpholinoethanol esters) of carboxyl functional groups, N-acyl derivatives (e.g., N-acetyl)N-Mannich bases, Schiff bases and enaminones of amino functional groups, oximes, acetals, ketals and enol esters of ketone and aldehyde functional groups in compounds described herein, and the like (e.g., Bundegaard, H. “Design of Prodrugs” p1-92, Elesevier, New York-Oxford (1985)).

The term “protecting group” refers to a grouping of atoms that when attached to a reactive group in a molecule masks, reduces or prevents that reactivity. Examples of protecting groups can be found in Green and Wuts, Protective Groups in Organic Chemistry, (Wiley, 2^{nd} ed. 1991); Harrison and Harrison et al., Compendium of Synthetic Organic Methods, Vols. 1-8 (John Wiley and Sons, 1971-1996); and Kocienski, Protecting Groups, (Verlag, 3^{rd} ed. 2003).

The term “amine protecting group” refers to a functional group that converts an amine, amide, or other nitrogen-containing moiety into a different chemical group that is substantially inert to the conditions of a particular chemical reaction. Amine protecting groups can be removed easily and selectively in good yield under conditions that do not affect other functional groups of the molecule. Examples of amine protecting groups include, but are not limited to, formyl, acetyl, benzyl, t-butyldimethylsilyl, t-butdyldiphenylsilyl, t-butyloxycarbonyl (Boc), p-methoxybenzyl, methoxymethyl, tosyl, trifluoroacetyl, trimethylsilyl (TMS), fluorenyl-methyloxycarbonyl, 2-trimethylsilyl-ethyoxycarbonyl, 1-methyl-1-(4-biphenylyl) ethoxycarbonyl, allyloxycarbonyl, benzyloxycarbonyl (CBZ), 2-trimethylsilyl-ethanesulfonyl (SES), trityl and substituted trityl groups, 9-fluorenylmethyloxycarbonyl (FMOC), nitro-veratryloxycarbonyl (NVOC), and the like. Other amine protecting groups can be identified by those of skill in the art.

Representative hydroxy protecting groups include those where the hydroxy group is either acylated or alkylated such as benzyl, and trityl ethers as well as alkyl ethers, tetrahydropyranyl ethers, trialkylsilyl ethers and allyl ethers.

Additionally, the salts of the compounds described herein, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

The term “solvates” refers to solvent addition forms that contain either stoichiometric or non stoichiometric amounts of solvent. Some compounds have a tendency to trap a fixed molar ratio of solvent molecules in the crystalline solid state, thus forming a solvate. If the solvent is water, the solvate formed is a hydrate; when the solvent is alcohol, the solvate formed is an alcoholate. Hydrates are formed by the combination of one or more molecules of water with one of the substances in which the water retains its molecular state as H₂O, such combination being able to form one or more hydrate.

The compounds, salts and prodrugs described herein can exist in several tautomeric forms, including the enol and imine form, and the keto and enamine form and geometric isomers and mixtures thereof. All such tautomeric forms are included within the scope of the present invention. Tautomers exist as mixtures of a tautomeric set in solution. In solid form, usually one tautomer predominates. Even though one tautomer may be described, the present application includes all tautomers of the present compounds. A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs.

Tautomerizations can be catalyzed by: Base: 1. deprotonation; 2. formation of a delocalized anion (e.g., an enolate); 3. protonation at a different position of the anion; Acid: 1. protonation; 2. formation of a delocalized cation; 3. deprotonation at a different position adjacent to the cation.

The term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

A “patient,” “subject,” or “host” to be treated by the subject method may mean either a human or non-human animal, such as primates, mammals, and vertebrates.

The term “prophylactic or therapeutic” treatment refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent”, “drug”, “medicament” and “bioactive substance” refer to molecules and other agents that are biologically, physiologically, or pharmacologically active substances that act locally or systemically in a patient or subject to treat a disease or condition, such as retinal degeneration or other forms of retinal disease whose etiology involves elevated levels of all trans-retinal in the ocular tissue of a subject. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer, the identity of the agent; the mode and method of administration; and any other materials incorporated in the polymer matrix in addition to the agent.

The term “ED50” refer to the dose of a drug, which produces 50% of its maximum response or effect, or alternatively, the dose, which produces a pre-determined response in 50% of test subjects or preparations. The term “LD50” refers to the dose of a drug, which is lethal in 50% of test subjects. The term “therapeutic index” refers to the therapeutic index of a drug, defined as LD50/ED50.

The term “substituted,” as used herein, means that any one or more hydrogens on the designated atom is replaced with a selection from the indicated group, provided that the designated atom's normal valency is not exceeded, and that the substitution results in a stable compound. When the substituent is keto (i.e., ═O), then 2 hydrogens on the atom are replaced. Ring double bonds, as used herein, are double bonds that are formed between two adjacent ring atoms (e.g., C═C, C═N, or N═N).

With respect to any chemical compounds, the present application is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

When a bond to a substituent is shown to cross a bond connecting two atoms in a ring, then such substituent can be bonded to any atom in the ring. When a substituent is listed without indicating the atom via which such substituent is bonded to the rest of the compound of a given formula, then such substituent can be bonded via any atom in such substituent. Combinations of substituents and/or variables are permissible, but only if such combinations result in stable compounds.

When an atom or a chemical moiety is followed by a subscripted numeric range (e.g., C₁₋₆), the invention is meant to encompass each number within the range as well as all intermediate ranges. For example, “C₁₋₆ alkyl” is meant to include alkyl groups with 1, 2, 3, 4, 5, 6, 1-6, 1-5, 1-4, 1-3, 1-2, 2-6, 2-5, 2-4, 2-3, 3-6, 3-5, 3-4, 4-6, 4-5, and 5-6 carbons.

As used herein, “alkyl” is intended to include both branched (e.g., isopropyl, tert-butyl, isobutyl), straight-chain e.g., methyl, ethyl, propyl, butyl, pentyl, hexyl, heptyl, octyl, nonyl, decyl), and cycloalkyl (e.g., alicyclic) groups (e.g., cyclopropyl, cyclopentyl, cyclohexyl, cycloheptyl, cyclooctyl), alkyl substituted cycloalkyl groups, and cycloalkyl substituted alkyl groups. Such aliphatic hydrocarbon groups have a specified number of carbon atoms. For example, C₁₋₄ alkyl is intended to include C₁, C₂, C₃, C₄, C₅, and C₆ alkyl groups. As used herein, “lower alkyl” refers to alkyl groups having from 1 to 6 carbon atoms in the backbone of the carbon chain. “Alkyl” further includes alkyl groups that have oxygen, nitrogen, sulfur or phosphorous atoms replacing one or more hydrocarbon backbone carbon atoms. In certain embodiments, a straight chain or branched chain alkyl has six or fewer carbon atoms in its backbone (e.g., C₁-C₆ for straight chain, C₃-C₆ for branched chain), for example four or fewer. Likewise, certain cycloalkyls have from three to eight carbon atoms in their ring structure, such as five or six carbons in the ring structure.

The term “substituted alkyls” refers to alkyl moieties having substituents replacing a hydrogen on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “aralkyl” moiety is an alkyl substituted with an aryl (e.g., phenylmethyl (benzyl)).

Furthermore, “alkyl” is intended to include moieties which are diradicals, i.e., having two points of attachment. A nonlimiting example of such an alkyl moiety that is a diradical is —CH₂CH₂—, i.e., a C₂ alkyl group that is covalently bonded via each terminal carbon atom to the remainder of the molecule.

As used herein, “halo” or “halogen” refers to fluoro, chloro, bromo, and iodo. “Counterion” is used to represent a small, negatively charged species such as fluoride, chloride, bromide, iodide, hydroxide, acetate, and sulfate.

“Stable compound” and “stable structure” are meant to indicate a compound that is sufficiently robust to survive isolation, and as appropriate, purification from a reaction mixture, and formulation into an efficacious therapeutic agent.

“Free compound” is used herein to describe a compound in the unbound state.

In the specification, the singular forms also include the plural, unless the context clearly dictates otherwise. Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

“Small molecule” refers to a molecule, which has a molecular weight of less than about 2000 amu, or less than about 1000 amu, and even less than about 500 amu.

All percentages and ratios used herein, unless otherwise indicated, are by weight.

The term “retina” refers to a region of the central nervous system with approximately 150 million neurons. It is located at the back of the eye where it rests upon a specialized epithelial tissue called retinal pigment epithelium or RPE. The retina initiates the first stage of visual processing by transducing visual stimuli in specialized neurons called “photoreceptors”. Their synaptic outputs are processed by elaborate neural networks in the retina and then transmitted to the brain. The retina has evolved two specialized classes of photoreceptors to operate under a wide range of light conditions. “Rod” photoreceptors transduce visual images under low light conditions and mediate achromatic vision. “Cone” photoreceptors transduce visual images in dim to bright light conditions and mediate both color vision and high acuity vision.

Every photoreceptor is compartmentalized into two regions called the “outer” and “inner” segment. The inner segment is the neuronal cell body containing the cell nucleus. The inner segment survives for a lifetime in the absence of retinal disease. The outer segment is the region where the light sensitive visual pigment molecules are concentrated in a dense array of stacked membrane structures. Part of the outer segment is routinely shed and regrown in a diurnal process called outer segment renewal. Shed outer segments are ingested and metabolized by RPE cells.

The term “macula” refers to the central region of the retina, which contains the fovea where visual images are processed by long slender cones in high spatial detail (“visual acuity”). “Macular degeneration” is a form of retinal neurodegeneration, which attacks the macula and destroys high acuity vision in the center of the visual field. AMD can be in a “dry form” characterized by residual lysosomal granules called lipofuscin in RPE cells, and by extracellular deposits called “drusen”. Drusen contain cellular waste products excreted by RPE cells. “Lipofuscin” and drusen can be detected clinically by ophthalmologists and quantified using fluorescence techniques. They can be the first clinical signs of macular degeneration.

Lipfuscin contains aggregations of A2E. Lipofuscin accumulates in RPE cells and poisons them by multiple known mechanisms. As RPE cells become poisoned, their biochemical activities decline and photoreceptors begin to degenerate. Extracellular drusen may further compromise RPE cells by interfering with their supply of vascular nutrients. Drusen also trigger inflammatory processes, which leads to choroidal neovascular invasions of the macula in one patient in ten who progresses to wet form AMD. Both the dry form and wet form progress to blindness.

The term “ERG” is an acronym for electroretinogram, which is the measurement of the electric field potential emitted by retinal neurons during their response to an experimentally defined light stimulus. ERG is a non-invasive measurement, which can be performed on either living subjects (human or animal) or a hemisected eye in solution that has been removed surgically from a living animal.

Embodiments described herein relate to compounds for use in the treatment of ocular disorders, such as ocular diseases and disorders related to aberrant all-trans-retinal accumulation in a subject's ocular tissue. It has been discovered that all-trans-retinal, a retinoid metabolite naturally produced during visual processing, is highly toxic when present at elevated levels. To lower its toxicity, therapeutic compounds have been identified below that can be delivered to and retained in the eye to modulate the visual (retinoid) cycle.

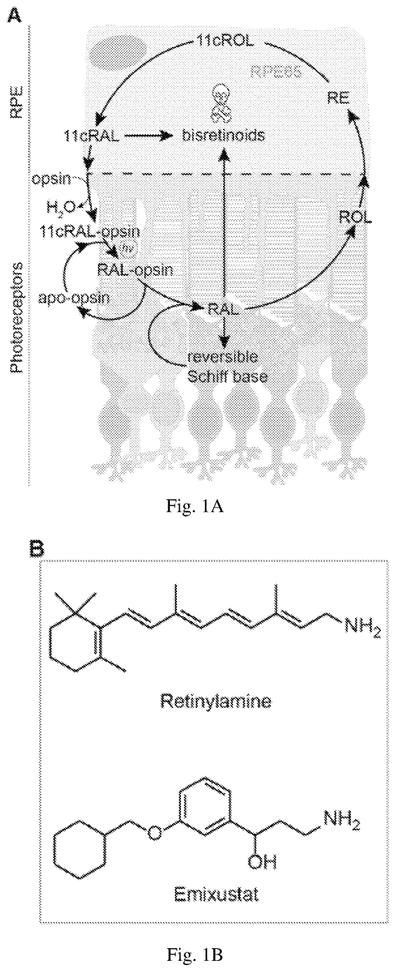

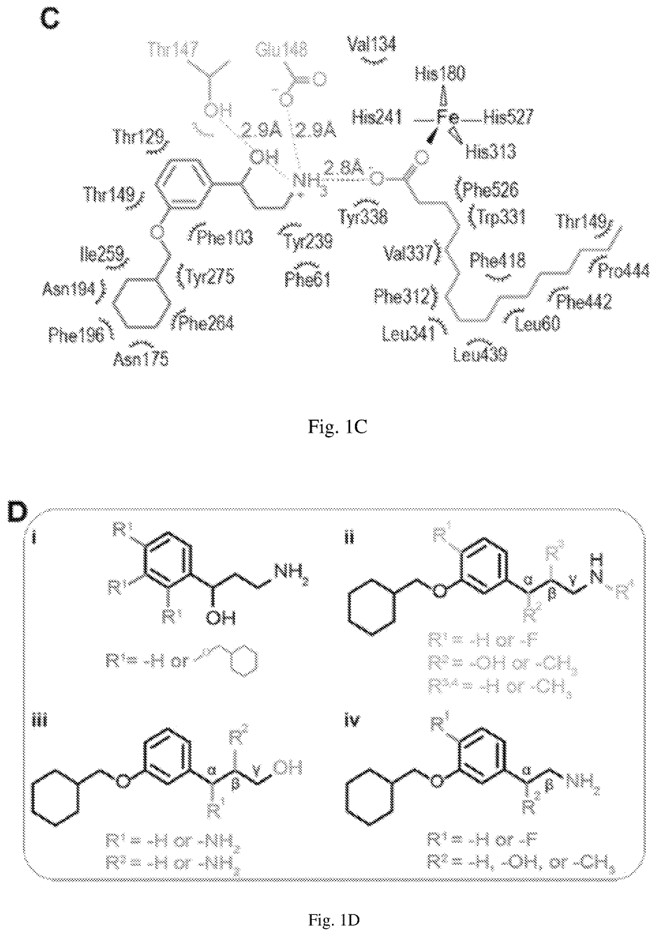

The concerted work of two molecular pathways is at the base of vision. First, phototransduction amplifies the signal derived from the capture of photons by a chromophore, 11-cis-retinal (11cRAL), covalently bound to visual opsins in the rod and cone photoreceptor cells. Second, the (retinoid) visual cycle, a multienzymatic process based largely within the retinal pigment epithelium (RPE), regenerates the spent chromophore, all-trans-retinal (RAL), to its 11-cis configuration (FIG. 1A). In addition, an incompletely defined cone/Müller cell pathway and the retinal G-coupled protein receptor (RGR)-mediate nonclassical visual cycle activity that also supports rod and cone function.

The classical visual cycle is essential to sustain phototransduction over time; however, compelling data indicate that this pathway can also drive disease progression in specific retinopathies including Stargardt disease (STGD), diabetic retinopathy (DR), light-induced retinopathy, and potentially age-related macular degeneration (AMD). Indeed, genetic ablation of essential enzymes and retinoid-binding proteins of the visual cycle, or their inhibition, protects the retina from degeneration induced by light.

In STGD and AMD, it is thought that the retinoid processing capability of the visual cycle may be overwhelmed as a consequence of prolonged exposure to light and/or attenuated metabolic state, hence allowing unbound RAL to form bisretinoids (e.g., A2E). The accumulation of bisretinoids in lipofuscin granules within the RPE is believed to be detrimental for the retina, even though they may also represent photoreceptor detoxification products. In the metabolically dysregulated diabetic eye, partial or total blockade of the visual cycle results in improvement of key markers of retinal stress likely due to modulation of photoreceptor activity.

Central to the economy of the visual cycle is an enzyme known as RPE 65 kDa protein (RPE65) (FIG. 1A). To date, RPE65 is the only retinoid isomerase known to cleave and isomerize all-trans-retinyl esters (RE) to produce 11-cis-retinol (11cROL), the precursor of 11cRAL. RPE65 is an obligate component of the visual cycle, and thus it stands as a key target for pharmacological inhibition in visual cycle-associated diseases. Rpe65 deficient mice are characterized by the absence of ocular 11-cis-retinoids resulting in dramatically impaired vision. Clinically, defects in the Rpe65 gene are associated with blinding diseases resulting from the absence of 11cRAL in the retina. As such, any effort to therapeutically inhibit RPE65 must avoid inducing diseases associated with visual chromophore deficiency. Thus, developing visual cycle inhibitors with ideal pharmacokinetic properties is crucial.

The first targeted pharmacological inhibitors of RPE65 were designed based on the hypothesis that the trans to cis isomerization reaction occurs through a carbocation intermediate. These inhibitors consisted of positively charged, amine derivatives of retinol that mimic the transition state of the retinoid isomerization reaction, with retinylamine being the most potent among them (FIG. 1B). A concern that metabolites of retinylamine may activate the receptors of retinoic acid together with the need to improve the solubility of these molecules prompted the development of inhibitors with a non-retinoid scaffold. Emixustat, first reported in 2012 by Acucela, is the best characterized RPE65 inhibitor of this second class. This compound is 10 times more potent than retinylamine in vitro. It is orally bioavailable with good ocular distribution and selectively inhibits the visual cycle as demonstrated in phase I and II clinical trials. In a phase III trial for AMD associated with geographic atrophy, emixustat therapy did not outperform the placebo control.

Persistent suppression of RPE65 leads to problematic night blindness (nyctalopia) and thus narrows the therapeutic window of emixustat. The γ-amino-α-aryl alcohol moiety of emixustat mediates crucial interactions with the residues Thr147 and Glu148 in the active site of RPE65 (FIG. 1C), but is also susceptible to rapid metabolism by a primary amine oxidase enzyme known as vascular adhesion protein 1 (VAP-1). Consequently, the critical barriers to generating clinically effective RPE65 inhibitors lie in finely tuning the metabolic aspects of their pharmacokinetic profile.

In some embodiments, the compounds described herein have strategically incorporated deuterium and/or fluorine to modulate the compounds' potency and metabolism. Previously we showed that selective fluorination of emixustat could be used to abrogate phase-I hydroxylation events. Moreover, these studies partially addressed the metabolic liability of emixustat by deuterating the carbon a to the primary amine, but did not ablate it.

Regioselective incorporation of fluorine can impact pKa modulation; alter target selectivity through conformational variations or changes in specific hydrophobic interactions; and alter tissue-specific penetration (e.g., central nervous system (CNS)), through modification of lipophilicity. These effects of fluorination are in addition to the well-established strategy of replacing metabolically labile hydrogens with C—F bonds. Regioselective incorporation of deuterium can be used to attenuate amine oxidation and rapid metabolic elimination via engineering a localized primary isotope effect. Collectively, the compounds have improved potency, absorption, selectivity, and metabolism to mitigate the toxicity of all-trans-retinal in age-related blindness.

In some embodiments, the ocular disorder associated with aberrant all-trans-retinal accumulation in the ocular tissue of a subject can include, for example, retinal degeneration, macular degeneration, including age-related macular degeneration, such as the dry form and the wet form of age related macular degeneration, Stargardt's disease, Stargardt macular degeneration, fundus flavimaculatus, geographic atrophy, retinitis pigmentosa, ABCA4 mutation related retinal dystrophies, vitelliform (or Best) macular degeneration, adult onset form of vitelliform macular dystrophy, Sorsby's fundus dystrophy, Malattia leventinese (Doyne honeycomb or dominant radial drusen), diabetic retinopathy, diabetic maculopathy, diabetic macular edema, retinopathy that is or presents geographic atrophy and/or photoreceptor degeneration, retinopathy that is a lipofuscin-based retinal degeneration, aberrant modulation of lecithin-retinol acyltransferase in an eye, Leber's congenital amaurosis, retinal detachment, hemorrhagic retinopathy, hypertensive retinopathy, hereditary or non hereditary optic neuropathy, inflammatory retinal disease, retinal blood vessel occlusion, retinopathy of prematurity, ischemia reperfusion related retinal injury, proliferative vitreoretinopathy, retinal dystrophy, uveitis, retinal disorders associated with Alzheimer's disease, retinal disorders associated with multiple sclerosis, retinal disorders associated with Parkinson's disease, retinal disorders associated with viral infection (cytomegalovirus or herpes simplex virus), retinal disorders related to light overexposure or myopia, retinal disorders associated with AIDS, glaucoma, genetic retinal dystrophies, traumatic injuries to the optic nerve, such as by physical injury, excessive light exposure, or laser light, neuropathies due to a toxic agent or caused by adverse drug reactions or vitamin deficiency, progressive retinal atrophy or degeneration, retinal diseases or disorders resulting from mechanical injury, chemical or drug-induced injury, thermal injury, radiation injury, light injury, or laser injury, hereditary and non-hereditary retinal dystrophy, ophthalmic injuries from environmental factors, such as light-induced oxidative retinal damage, laser-induced retinal damage, “flash bomb injury,” or “light dazzle”, refractive errors including but not limited to myopia, and retinal diseases related to A2E accumulation including RDS/PHRP2-related macular degeneration, Batten disease (juvenile neuronal ceroid lipofuscinosis), and central serous chorioretinopathy.

In some embodiments, the compounds described herein inhibit RPE65 enzymatic activity involved in retinoid metabolism in the eye of the subject. In certain embodiments, the compounds for use in a method described herein inhibit or at least partially inhibit RPE65 and but do not cause delayed dark adaptation (i.e., night blindness) in a subject.

In some embodiments, the pharmacokinetics of the compound upon administration to the subject is such that the compound does not promote night blindness when administered daily.

In other embodiments, the compound is not deaminated by VAP-1 enzyme.

In some embodiments, the compounds, which inhibit retinal degeneration upon administration to a subject, can be selected using an in vitro assays that measure the ability of a compound to inhibit retinoid isomerase activity in the ocular tissue and in vivo assays that measure chromophore regeneration and ERG as well as the optical coherence tomography score of retinas of Rdh8^{-/-}Abca4^{-/-} mice exposed to intense light-induced retinal degeneration.

In certain embodiments, the compounds, which inhibit retinal degeneration upon administration to a subject, at least partially inhibit RPE65 activity in a subject's ocular tissue. In some embodiments, the compounds when administered to a Rdh8^{-/-}Abca4^{-/-} mouse increase the optical coherence tomography score of the mouse in comparison to untreated control animal. In other embodiments, therapeutic efficacy of the compounds described can be determined using an in vitro assay that measures the ability of a compound to improve viability of RPE cells treated with retinal.

In some embodiments, a compound for use in a method described herein can include the formula (I):

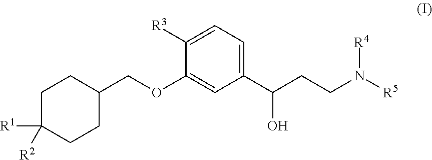

or a pharmaceutically acceptable salt, tautomer, or solvate thereof, wherein:
R¹, R², and R³ are each independently H or F;
R⁴ and R⁵ are each independently H, D, or an alkyl, which is optionally substituted with one or more D or F, and wherein at least one of R⁴ or R⁵ is an alkyl, which is optionally substituted with one or more D or F.

In some embodiments, at least one of R¹, R², or R³ is F or at least one of R⁴ or R⁵ is an alkyl substituted with one or more D or F.

In some embodiments, at least one of R¹ or R² is F.

In other embodiments, both of R¹ and R² are F.

In some embodiments, R³ is F.

In some embodiments, at least one R⁴ or R⁵ is an alkyl substituted with one or more D or F.

In other embodiments, at least one R⁴ or R⁵ is C₁-C₆ alkyl substituted with one or more D or F.

In other embodiments, both of R⁴ and R⁵ are C₁-C₆ alkyl substituted with one or more D or F.

In some embodiments, at least one R⁴ or R⁵ is methyl substituted with one or more D or F.

In other embodiments, both of R⁴ and R⁵ are methyl substituted with one or more D or F.

In still other embodiments, at least one R⁴ or R⁵ is —CD₃ or —CF₃.

In yet other embodiments, both of R⁴ and R⁵ are —CD₃ or —CF₃.

Other embodiments relate to a compound selected from:

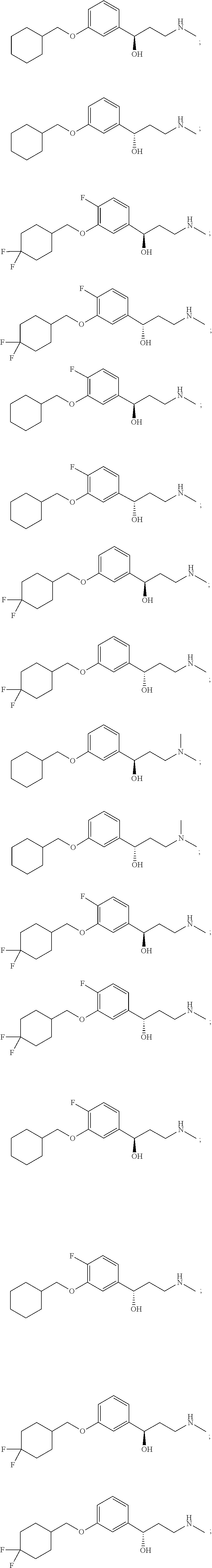

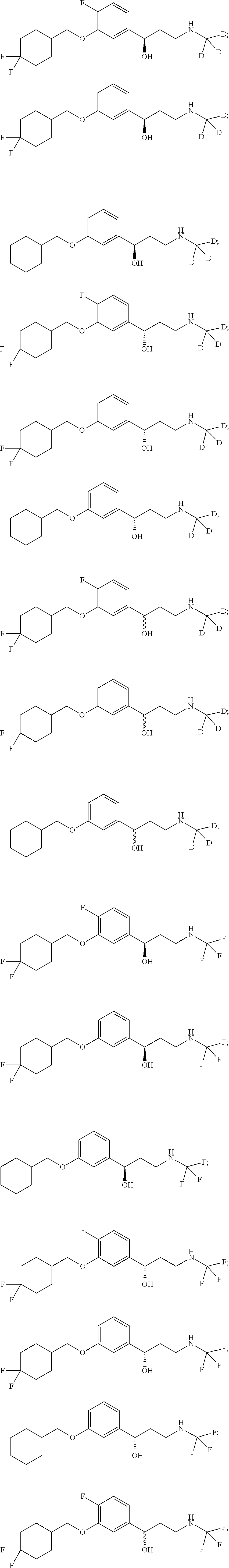

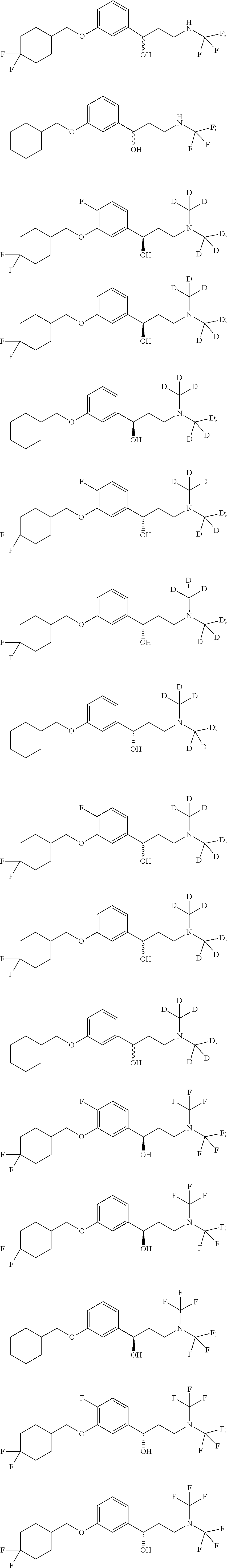

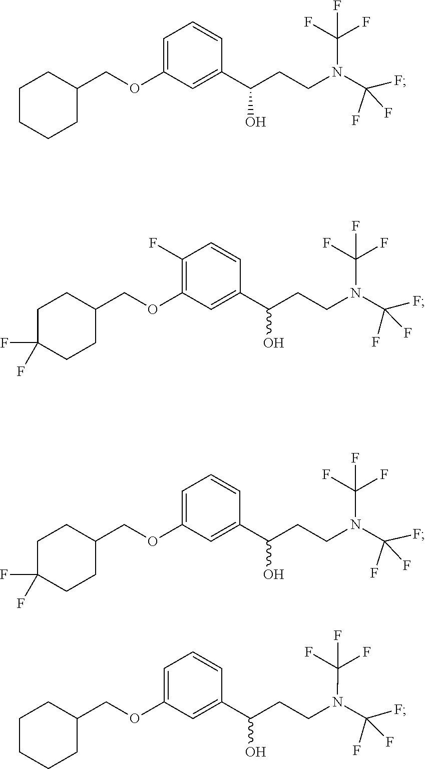 [SMILES: O[C@@H](CCN(C(F)(F)F)C(F)(F)F)c1cccc(OCC2CCCCC2)c1 ; OC(CCN(C(F)(F)F)C(F)(F)F)c1ccc(F)c(OCC2CCC(F)(F)CC2)c1 ; OC(CCN(C(F)(F)F)C(F)(F)F)c1cccc(OCC2CCC(F)(F)CC2)c1 ; OC(CCN(C(F)(F)F)C(F)(F)F)c1cccc(OCC2CCCCC2)c1]

or a pharmaceutically acceptable salt, tautomer, or solvate thereof.

The compounds used in the methods described herein can be administered to the subject to treat the ocular disorder (e.g., macular degeneration or Stargardt disease) using standard delivery methods including, for example, ophthalmic, topical, parenteral, subcutaneous, intravenous, intraarticular, intrathecal, intramuscular, intraperitoneal, intradermal injections, or by transdermal, enteral, buccal, oromucosal, oral routes or via inhalation. Preferably, the compounds described herein can be administered enterally, such as orally. The particular approach and dosage used for a particular subject depends on several factors including, for example, the general health, weight, and age of the subject. Based on factors such as these, a medical practitioner can select an appropriate approach to treatment.

Generally, the effective amount of the compound may be in the range of about 1 to about 1,000 mg in the oral administration, about 0.1 to about 500 mg in the intravenous administration, about 5 to about 1,000 mg in the topical administration. Generally, the daily dosage for adults is in the range of about 0.1 to about 5,000 mg, preferably about 1 to about 1,000 mg but cannot be determined uniformly because it depends on age, sex, body weight and the physical condition of the patients to be treated. The formulation may be administered once a day or several times a day with a divided dose.

Treatment according to the method described herein can be altered, stopped, or re-initiated in a subject depending on the status of ocular disorder. Treatment can be carried out as intervals determined to be appropriate by those skilled in the art. For example, the administration can be carried out 1, 2, 3, or 4 times a day. In another embodiment, the compound can be administered after induction of macular degeneration has occurred.

The treatment methods can include administering to the subject a therapeutically effective amount of the compound. Determination of a therapeutically effective amount is within the capability of those skilled in the art. The exact formulation, route of administration, and dosage can be chosen by the individual physician in view of the subject's condition.

Formulation of pharmaceutical compounds for use in the modes of administration noted above (and others) are described, for example, in Remington's Pharmaceutical Sciences (18th edition), ed. A. Gennaro, 1990, Mack Publishing Company, Easton, Pa. (also see, e.g., M. J. Rathbone, ed., Oral Mucosal Drug Delivery, Drugs and the Pharmaceutical Sciences Series, Marcel Dekker, Inc., N.Y., U.S.A., 1996; M. J. Rathbone et al., eds., Modified-Release Drug Delivery Technology, Drugs and the Pharmaceutical Sciences Series, Marcel Dekker, Inc., N.Y., U.S.A., 2003; Ghosh et al., eds., Drug Delivery to the Oral Cavity, Drugs and the Pharmaceutical Sciences Series, Marcel Dekker, Inc., N.Y. U.S.A., 1999.

In one example, the compound can be provided in an ophthalmic preparation that can be administered to the subject's eye. The ophthalmic preparation can contain the compound in a pharmaceutically acceptable solution, suspension or ointment. Some variations in concentration will necessarily occur, depending on the particular compound employed, the condition of the subject to be treated and the like, and the person responsible for treatment will determine the most suitable concentration for the individual subject. The ophthalmic preparation can be in the form of a sterile aqueous solution containing, if desired, additional ingredients, for example, preservatives, buffers, tonicity agents, antioxidants, stabilizers, nonionic wetting or clarifying agents, and viscosity increasing agents.

Examples of preservatives for use in such a solution include benzalkonium chloride, benzethonium chloride, chlorobutanol, thimerosal and the like. Examples of buffers include boric acid, sodium and potassium bicarbonate, sodium and potassium borates, sodium and potassium carbonate, sodium acetate, and sodium biphosphate, in amounts sufficient to maintain the pH at between about pH 6 and about pH 8, and for example, between about pH 7 and about pH 7.5. Examples of tonicity agents are dextran 40, dextran 70, dextrose, glycerin, potassium chloride, propylene glycol, and sodium chloride.

Examples of antioxidants and stabilizers include sodium bisulfite, sodium metabisulfite, sodium thiosulfite, and thiourea. Examples of wetting and clarifying agents include polysorbate 80, polysorbate 20, poloxamer 282 and tyloxapol. Examples of viscosity-increasing agents include gelatin, glycerin, hydroxyethylcellulose, hydroxmethylpropylcellulose, lanolin, methylcellulose, petrolatum, polyethylene glycol, polyvinyl alcohol, polyvinylpyrrolidone, and carboxymethylcellulose. The ophthalmic preparation will be administered topically to the eye of the subject in need of treatment by conventional methods, for example, in the form of drops or by bathing the eye in the ophthalmic solution.

The compound can also be formulated for topical administration through the skin. “Topical delivery systems” also include transdermal patches containing the ingredient to be administered. Delivery through the skin can further be achieved by iontophoresis or electrotransport, if desired.

Formulations for topical administration to the skin can include, for example, ointments, creams, gels and pastes comprising the retinal sequestering compound in a pharmaceutical acceptable carrier. The formulation of the compounds for topical use includes the preparation of oleaginous or water-soluble ointment bases, as is well known to those in the art. For example, these formulations may include vegetable oils, animal fats, and, for example, semisolid hydrocarbons obtained from petroleum. Particular components used may include white ointment, yellow ointment, cetyl esters wax, oleic acid, olive oil, paraffin, petrolatum, white petrolatum, spermaceti, starch glycerite, white wax, yellow wax, lanolin, anhydrous lanolin and glyceryl monostearate. Various water-soluble ointment bases may also be used, including glycol ethers and derivatives, polyethylene glycols, polyoxyl 40 stearate and polysorbates.

Subjects affected with or at risk of macular degeneration, which are not readily accessible or suitable for ophthalmic (e.g., eye-drops) and/or topical administration, can be treated by a systemic approach, such as oral, enteral, or intravenous infusion. For example, the compound can be administered at a low dosage by continuous intravenous infusion.

In another example, in which a patient requires longer-term care, the compound can be administered intermittently (e.g., every 12-24 hours). In a variation of this approach, the initial or loading dose can be followed by maintenance doses that are less than, (e.g., half) the loading dose or by continuous infusion. The duration of such treatment can be determined by those having skill in the art, based on factors, for example, the severity of the condition and the observation of improvements.

When administering the compound to the subject by intravenous infusion, devices and equipment (e.g., catheters, such as central or peripheral venous catheters, tubing, drip chambers, flashback bulbs, injection Y sites, stopcocks, and infusion bags) can be used that are compatible with the retinal sequestering compound.

As discussed above, the compounds may be administered to a subject in order to treat or prevent macular degeneration and other forms of retinal disease whose etiology involves elevated levels of toxic all-trans-retinal in a subject. Other diseases, disorders, or conditions characterized by increased or excessive all-trans-retinal in ocular tissue may be similarly treated.

In one embodiment, a subject is diagnosed as having symptoms of macular degeneration, and then a disclosed compound is administered. In another embodiment, a subject may be identified as being at risk for developing macular degeneration (risk factors include a history of smoking, age, female gender, and family history), and then a disclosed compound is administered. In another embodiment, a subject may have dry AMD in both eye, and then a disclosed compound is administered. In another embodiment, a subject may have wet AMD in one eye but dry AMD in the other eye, and then a disclosed compound is administered. In yet another embodiment, a subject may be diagnosed as having Stargardt disease and then a disclosed compound is administered. In another embodiment, a subject is diagnosed as having symptoms of other forms of retinal disease whose etiology involves increased all-trans-retinal in ocular tissue of a subject, and then the compound is administered. In another embodiment, a subject may be identified as being at risk for developing other forms of retinal disease whose etiology involves increased all-trans-retinal in ocular tissue, and then the disclosed compound is administered. In some embodiments, a compound is administered prophylactically. In some embodiments, a subject has been diagnosed as having the disease before retinal damage is apparent. In some embodiments, a human subject may know that he or she is in need of the macular degeneration treatment or prevention.

In some embodiments, a subject may be monitored for the extent of macular degeneration. A subject may be monitored in a variety of ways, such as by eye examination, dilated eye examination, fundoscopic examination, visual acuity test, and/or biopsy. Monitoring can be performed at a variety of times. For example, a subject may be monitored after a compound is administered. The monitoring can occur, for example, one day, one week, two weeks, one month, two months, six months, one year, two years, five years, or any other time period after the first administration of a compound. A subject can be repeatedly monitored. In some embodiments, the dose of a compound may be altered in response to monitoring.

In some embodiments, the disclosed methods may be combined with other methods for treating or preventing macular degeneration or other forms of retinal disease whose etiology involves increased all-trans-retinal accumulation in ocular tissue, such as photodynamic therapy. For example, a patient may be treated with more than one therapy for one or more diseases or disorders. For example, a patient may have one eye afflicted with dry form AMD, which is treated with a compound described herein, and the other eye afflicted with wet form AMD, which is treated with, e.g., photodynamic therapy.

In yet another embodiment, the compound described herein can be administered as part of a combinatorial therapy with additional therapeutic agents. The phrase “combinatorial therapy” or “combination therapy” embraces the administration of a compound, and one or more therapeutic agents as part of a specific treatment regimen intended to provide beneficial effect from the co-action of these therapeutic agents. Administration of these therapeutic agents in combination typically is carried out over a defined period (usually minutes, hours, days or weeks depending upon the combination selected). “Combinatorial therapy” or “combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example by administering to the subject an individual dose having a fixed ratio of each therapeutic agent or in multiple, individual doses for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissue. The therapeutic agents can be administered by the same route or by different routes. The sequence in which the therapeutic agents are administered is not narrowly critical.

The invention is further illustrated by the following example, which is not intended to limit the scope of the claims.

### EXAMPLE

This example delineates the structural requirements of the γ-amino-α-aryl alcohol (what we have termed the RPE65 recognition element) to maintain high affinity for RPE65 active site while circumventing VAP-1-associated metabolic liability. We generated a library of 15 compounds (FIG. 1D) to systematically test for RPE65 inhibition in vitro. The best inhibitors were tested in vivo by monitoring the kinetics of the visual cycle in response to a short period of bright light exposure. The most potent compound was subsequently tested for susceptibility to VAP-1 and cytochrome P-450 (CYP-450)-mediated clearance in vitro and its in vivo ocular distribution and serum levels were profiled. We also report detailed structural information on its complex with RPE65.

### General Synthetic Methods

All reactions were performed in oven-dried glassware under inert atmosphere. Diisopropylamine was fractionally distilled in CaH₂ prior to use. All reagents purchased from various vendors (Sigma-Aldrich, Fisher, Oakwood, Combi Blocks, and AA Blocks) were used as supplied without purification. Thin-layer chromatography (TLC) was performed on 0.25 mm glass-backed EMD Millipore 60 F254 plates. Visualization of TLC plates was accomplished with UV light (254 nm), and final amines were stained with permanganate (KMnO₄). Purification of all intermediates was achieved by the use of the CombiFlash Nextgen 100 (Teledyne Isco). All final target amines were purified manually by forced air-flow on silica gel (Merck, 230-400 mesh) using eluting solvents (reported as VN ratio mixture), and finally filtered through sodium sulfate after solvents were removed by rotary evaporation. The ¹H, ¹³C, and F NMR nuclear magnetic resonance (NMR) spectra were recorded at 25° C. on a Bruker AVANCE NMR spectrometer operating at 500 MHz. ¹⁹F NMR spectra were recorded without decoupling from protons. Chemical shifts were reported in δ units, part per million, with reference to the residual solvent peak CDCl₃ (δ 7.26) and DMSO-d₄ (δ 2.50) for ¹H and CDCl₃ (δ 77.3) and DMSO-d₄ (δ 39.5) for ¹³C NMR spectra. NMR data are presented in the following order: chemical shift, peak multiplicity (s=singlet, d=doublet, t=triplet, q=quartet, m=multiplet, dd=doublet of doublet, dt=doublet of triplet, ddd=doublet of doublet of doublet, dq=doublet of quartet), coupling constant (in Hz). Analytical HPLC analysis of final targets was carried out on an Agilent 1260 series system consisting of a G4204A quaternary pump, a G4226A ALS auto-sampler, and a G1316C column compartment. The separation was performed on a Shimadzu Premier C18 (5 μm, 100 mm×4.5 mm) column using a mobile phase consisting of 0.1% formic acid in water (A) and 0.1% formic acid in acetonitrile (B) at a flow rate of 1 mL/min, and the mobile-phase gradients and time course were as follows: 0-5 min, 95% A/5% B: 5-10 min, 95-5% A/5-95% B; 10-20 min, 5% A/95% B; 20-25 min, 5-95% A/95-5% B; 25-30 min, 95% A/5% B. The final biologically tested compounds displayed≥95% purity except for 11d and 16b (>90%). Synthesis of the target emixustat amines is detailed below.

3-Amino-1-(4-(cyclohexylmethoxy)phenyl)propan-1-ol (3b). General Procedure 1. Compound 2b (300 mg, 1.2 mmol) in THF (1 mL) was added dropwise to an ice-cold stirred solution of lithium aluminum hydride (175 mg, 4.6 mmol) in THF (4 mL) under argon. The resulting mixture was stirred at 0° C. for 1 h and then was slowly quenched by the dropwise addition of water (300 μL), 15% NaOH (300 μL), and water (900 μL) consecutively. The reaction mixture was allowed to stir at room temperature (rt) for 0.5 h, filtered through cotton, diluted with water (30 mL), extracted with dichloromethane (2×10 mL), dried over sodium sulfate, and filtered. Following concentration in vacuo, the crude product was then purified by flash column chromatography giving a tan solid (144 mg, 47%). HPLC: t_{R}=10.0 min (95%). ¹H NMR (500 MHz, CDCl₃) δ 7.28 (d, J=8.7 Hz, 2H), 6.86 (d, J=8.7 Hz, 2H), 4.90 (dd, J=8.7, 3.3 Hz, 1H), 3.74 (d, J=6.4 Hz, 2H), 3.10 (ddd, J=12.5, 5.7, 4.2 Hz, 1H), 2.94 (ddd, J=12.7, 9.2, 3.9 Hz, 1H), 1.91-1.65 (m, 9H), 1.35-1.14 (m, 4H), 1.04 (qd, J=12.2, 3.4 Hz, 2H). ¹³C NMR (126 MHz, CDCl₃) δ 158.1, 137.1, 126.9, 114.4, 75.3, 73.6, 40.7, 39.9, 37.8, 30.0, 26.6, 25.9.

HRMS (ESI⁺): (m/z) calculated for C₁₆H₂₆NO₂ [M+H]⁺ 264.1958; found 264.1957.

3-Amino-1-(2-(cyclohexylmethoxy)phenyl)propan-1-ol (3c). General procedure 1 was followed using nitrile 2c (660 mg, 2.54 mmol) to give a yellow solid (294 mg, 44%). HPLC: t_{R}=9.8 min (97%). HPLC: t_{R}=9.630 min (97%). ¹H NMR (500 MHz, CDCl₃) δ 7.50 (dd, J=7.5, 1.8 Hz, 1H), 7.20 (td, J=7.7, 1.8 Hz, 1H), 6.96 (td, J=7.5, 1.1 Hz, 1H), 6.82 (dd, J=8.3, 1.1 Hz, 1H), 5.23 (dd, J=8.0, 3.5 Hz, 1H), 3.77 (qd, J=9.0, 6.0 Hz, 2H), 3.04-2.98 (m, 1H), 2.97-2.91 (m, 1H), 2.51 (s, 2H), 2.03-1.91 (m, 1H), 1.90-1.67 (m, 8H), 1.36-1.16 (m, 4H), 1.15-1.03 (m, 2H). ¹³C NMR (126 MHz, CDCl₃) δ 155.6, 133.2, 127.8, 126.6, 120.5, 110.9, 73.3, 70.5, 40.5, 38.4, 37.9, 30.1, 30.1, 26.6, 26.0. HRMS (ESI⁺): (m/z) calculated for C₁₆H₂₆NO₂ [M+H]⁺ 264.1958; found 264.1957.

3-Amino-3-(3-(cyclohexylmethoxy)phenyl)propan-1-ol (6a). General Procedure 2. Compound 5a (300 mg, 0.76 mmol) in THF (1 mL) was added dropwise to an ice-cold stirred solution of lithium aluminum hydride (175 mg, 4.6 mmol) in THF (4 mL) under argon. The resulting mixture was stirred at 0° C. for 1 h and then was slowly quenched by the dropwise addition of water (300 μL), 15% NaOH (300 μL), and water (900 μL) consecutively. The reaction mixture was allowed to stir at rt for 0.5 h, filtered through cotton, diluted with water (30 mL), extracted with dichloromethane (2×10 mL), and dried over sodium sulfate. Following filtration, solvents were removed in vacuo to give a colorless syrup (140 mg). This solid was dissolved in DCM (5 mL) and TFA (0.6 mL, 7.83 mmol) was added and stirred for 1 h at room temperature. The solution was basified with 15% NaOH (20 mL) and extracted with DCM (2×10 mL). The combined layers were dried over Na₂SO₄, concentrated under reduced pressure, and purified by flash column chromatography to finally give a colorless syrup (57 mg, 28%). HPLC: t_{R}^{=9.8} min (95%). ¹H NMR (500 MHz, CDCl₃) δ 7.24 (t, J=7.8 Hz, 1H), 6.89-6.84 (m, 2H), 6.81 (dd, J=7.6, 2.0 Hz, 1H), 4.17 (dd, J=9.6, 3.6 Hz, 1H), 3.87-3.77 (m, 2H), 3.73 (d, J=6.4 Hz, 2H), 3.35 (s, 3H), 2.08-1.97 (m, 1H), 1.93-1.82 (m, 3H), 1.81-1.66 (m, 4H), 1.34-1.16 (m, 3H), 1.04 (qd, J=12.3, 3.4 Hz, 2H). ¹³C NMR (126 MHz, CDCl₃) δ 159.9, 1.30.1, 118.1, 113.9, 112.4, 73.6, 61.8, 56.7, 38.0, 37.8, 30.0, 26.6, 25.9. HRMS (ESI): (m/z) calculated for C₁₆H₂₆NO₂ [M+H]⁺ 264.1964; found 264.1957.

2-Amino-3-(3-(cyclohexylmethoxy)phenyl)propan-1-ol (6b). The general procedure 2 was applied to compound 5b (250 mg, 0.63 mmol) giving a colorless syrup (56 mg, 27%). HPLC: t_{R}^{=9.9} min (97%). ¹H NMR (500 MHz, CDCl₃) δ 7.20 (t, J=7.8 Hz, 1H), 6.78-6.71 (m, 3H), 3.74 (d, J=6.4 Hz, 2H), 3.64 (dd, J=10.5, 4.0 Hz, 1H), 3.37 (dd, J=10.5, 7.2 Hz, 1H), 3.17-3.08 (m, 1H), 2.76 (dd, J=13.4, 5.2 Hz, 1H), 2.49 (dd, J=13.4, 8.7 Hz, 1H), 1.91-1.84 (m, 2H), 1.80-1.67 (m, 5H), 1.58 (s, 3H), 1.35-1.16 (m, 4H), 1.05 (qd, J=12.2, 3.4 Hz, 2H). ¹³C NMR (126 MHz, CDCl₃) δ 159.7, 140.3, 129.6, 121.4, 115.6, 112.4, 73.5, 66.7, 54.2, 41.3, 37.9, 30.0, 26.6, 25.9. HRMS (ESI): (m/z) calculated for C₁₆H₂₆NO₂ [M+H]⁺ 264.1964; found 264.1958.

2-Amino-3-(4-(cyclohexylmethoxy)phenyl)propan-1-ol (6c). Following the general procedure 2, reduction then deprotection of 5c (300 mg, 0.77 mmol) afforded a white solid (75 mg, 37%). HPLC: t_{R}^{=9.9} min (99%). ¹H NMR (500 MHz, CDCl₃) δ 7.08 (d, J=8.5 Hz, 2H), 6.84 (d, J=8.5 Hz, 2H), 3.73 (d, J=6.3 Hz, 2H), 3.64 (dd, J=10.7, 3.9 Hz, 1H), 3.37 (dd, J=10.6, 7.2 Hz, 1H), 3.11 (s, 1H), 2.73 (dd, J=13.7, 5.3 Hz, 1H), 2.48 (dd, J=13.6, 8.5 Hz, 1H), 1.87 (d, J=14.7 Hz, 2H), 1.76 (d, J=12.5 Hz, 4H), 1.25 (d, J=89.0 Hz, 4H), 1.04 (qd, J=12.3, 3.4 Hz, 2H). ¹³C NMR (126 MHz, CDCl₃) δ 158.1, 130.3, 130.2, 114.7, 73.7, 66.5, 54.4, 40.1, 37.8, 30.0, 26.6, 25.9. HRMS (ESI): (m/z) calculated for C₁₆H₂₆NO₂ [M+H]⁺ 264.1964; found 264.1961.

2-(3-(Cyclohexylmethoxy)phenyl)ethan-1-amine (11b). Nitrile 9a (2.0 g, 8.7 mmol) and borane dimethylsulfide complex (5 M in ether, 5.3 mL, 26.5 mmol) in THF (40 mL) was stirred at reflux for 4 h under argon. The resulting mixture was cooled in an ice bath and quenched with MeOH until no bubbling was observed. Solvents were removed by rotary evaporation, and residue was diluted in water (30 mL) and extracted with DCM (3×10 mL). The combined organic layers were dried over sodium sulfate, and solvents were removed under reduced pressure. Crude material was purified by flash column chromatography to finally give a colorless syrup (133 mg, 5%). HPLC: t_{R}^{=10.0} min (99%). ¹H NMR (500 MHz, CDCl₃) δ 7.20 (dd, J=9.0, 7.5 Hz, 1H), 6.84-6.67 (m, 3H), 3.74 (d, J=6.4 Hz, 2H), 2.97 (t, J=6.8 Hz, 2H), 2.72 (t, J=6.8 Hz, 2H), 1.91-1.83 (m, 2H), 1.82-1.65 (m, 4H), 1.45 (s, 4H), 1.34-1.17 (m, 3H), 1.05 (qd, J=12.3, 3.4 Hz, 2H). ¹³C NMR (126 MHz, CDCl₃) δ 159.6, 141.5, 129.5, 121.1, 115.3, 112.1, 73.5, 43.6, 40.3, 37.8, 30.0, 26.6, 25.9. HRMS (EI): (m/z) calculated for C₁₆H₂₆NO₂ [M+H]⁺ 233.1780; found 233.1779.

2-(3-(Cyclohexylmethoxy)phenyl)propan-1-amine (1c). Borane dimethylsulfide complex (5.0 M in THF, 1.0 mL, 5.0 mmol) was added to an ice-cold solution of nitrile 9b (400 mg, 1.6 mmol) in THF (5 mL). The mixture was heated under reflux for 1 h when the reaction was placed in an ice bath and quenched by the slow addition of methanol until bubbling ceased. Solvents were removed under reduced pressure, and the crude product was purified by flash column chromatography giving a colorless oil (163 mg, 40%). HPLC: t_{R}=10.0 min (96%). ¹H NMR (500 MHz, CDCl₃) δ 7.22 (t, J=8.0 Hz, 1H), 6.81-6.70 (m, 3H), 3.74 (d, J=6.4 Hz, 2H), 2.84 (d, J=6.9 Hz, 2H), 2.71 (h, J=7.0 Hz, 1H), 1.92-1.84 (m, 2H), 1.82-1.65 (m, 4H), 1.36-1.27 (m, 2H), 1.24 (d, J=6.8 Hz, 3H), 1.21-1.11 (m, 2H), 1.10-1.00 (m, 2H). ¹³C NMR (126 MHz, CDCl₃) δ 159.6, 146.8, 129.5, 119.6, 114.0, 112.0, 73.5, 49.6, 43.8, 37.9, 30.1, 26.6, 25.9, 19.4. HRMS (ES): (m/z) calculated for C₁₆H₂₅NO [M]⁺ 247.1936; found 247.1933.

2-(3-(Cyclohexylmethoxy)-4-fluorophenyl)ethan-1-amine (11d). Nitro compound 10 (414 mg, 1.49 mmol) and lithium aluminum hydride (1 M in THF, 5.96 mL, 5.96 mmol) in THF (15 mL) were added to a 200 mL sealed tube to react 24 h at 50° C. with stirring. After cooling to room temperature, the reaction mixture was quenched with 20 mL of methanol until bubbling ceased. Solvents were removed under reduced pressure, and the crude product was purified by flash column chromatography giving a yellow oil (297 mg, 80%). HPLC: t_{R}=10.0 min (94%). ¹H NMR (500 MHz, CD₃OD) δ 7.03 (dd, J=11.2, 8.0 Hz, 1H), 6.96 (d, J=10.0 Hz, 1H), 6.80-6.76.18 (m, 1H), 3.86 (d, J=6.5 Hz, 2H), 2.91 (t, J=14.5 Hz, 2H), 2.76 (t, J=14.5 Hz, 2H), 1.94-1.74 (m, 6H), 1.42-1.10 (m, 5H). ¹³C NMR (126 MHz, CD₃OD) δ 153.8, 151.8, 148.7, 148.6, 137.5, 137.5, 122.0, 122.0, 116.9, 116.7, 116.7, 75.8, 44.3, 39.8, 39.3, 31.0, 27.8, 27.1. ¹⁹F NMR (471 MHz, CD₃OD) δ-140.08 HRMS (ES): (m/z) calculated for C₁₅H₂₂FNO [M]⁺ 251.1686; found 251.1689.

3-(3-(Cyclohexylmethoxy)phenyl)butan-1-amine (16b). Unsaturated nitrile 14 (818 mg, 3.2 mmol) and lithium aluminum hydride (1 M in THF, 16 mL, 16 mmol) in THF (40 mL) was added to a 200 mL sealed tube to react for 24 h at 80° C. with stirring. After cooling to room temperature, the reaction mixture was quenched with methanol (80 mL) until bubbling ceased. Solvents were removed under reduced pressure, and the crude product was purified by flash column chromatography giving a yellow oil (225 mg, 27%). HPLC: t_{R}=10.0 min (91%). ¹H NMR (500 MHz, CD₃OD) δ 7.17 (t, J=16.0 Hz, 1H), 6.77 (d, J=7.5 Hz, 1H), 6.75 (t, J=4.5 Hz, 1H), 3.75 (d, J=6.5 Hz, 2H), 2.77-2.70 (m, 1H), 2.57-2.45 (m, 2H), 1.90-1.71 (m, 8H), 1.38-1.06 (m, 8H). ¹³C NMR (126 MHz, CD₃OD) S 161.1, 150.1, 130.5, 120.3, 114.5, 113.0, 74.5, 42.5, 41.1, 39.4, 39.2, 31.2, 27.8, 27.2, 23.2. HRMS (ES): (m/z) calculated for C₁₇H₇NO [M]⁺ 261.2093; found 261.2090.

(3-(Cyclohexylmethoxy)phenyl)-2-methylpropan-1-amine (16c). Unsaturated nitrile 15a (458 mg, 1.8 mmol) and lithium aluminum hydride (1 M in THF, 9 mL, 9 mmol) in THF (20 mL) were added to a 200 mL sealed tube to react 24 h at 80° C. with stirring. After cooling to room temperature, the reaction mixture was quenched by methanol (40 mL) until bubbling ceased. Solvents were removed under reduced pressure, and the crude product was purified by flash column chromatography giving a colorless oil (88 mg, 19%). HPLC: t_{R}=9.8 min (96%). ¹H NMR (500 MHz, CD₃OD) δ 7.14 (t, J=16.0 Hz, 1H), 6.80-6.66 (m, 3H), 3.72 (d, J=6.5 Hz, 2H), 2.76-2.58 (m, 2H), 2.47-2.30 (m, 2H), 1.87-1.69 (m, 8H), 1.39-1.03 (m, 6H), 0.88 (d, J=6.5 Hz, 3H). ¹³C NMR (126 MHz, CD₃OD) S 160.9, 143.7, 130.3, 122.5, 116.6, 113.0, 74.5, 42.2, 39.3, 39.0, 31.1, 27.8, 27.1, 17.9. HRMS (ES): (m/z) calculated for C₁₇H₂₇NO [M]⁺ 261.2093; found 261.2093.

3-(3-(Cyclohexylmethoxy)-4-fluorophenyl)-2-methylpropan-1-amine (16d). Unsaturated nitrile 15b (805 mg, 2.95 mmol), palladium on activated carbon (161 mg, 20 wt %), and MeOH (20 mL) were added to a 50 mL round-bottom flask. The flask was placed in a high-pressure reaction vessel and hydrogenated (200 psi) at room temperature for 36 h with stirring. The reaction mixture was filtered, concentrated under reduced pressure, and purified by flash column chromatography giving a pale-yellow oil (320 mg, yield 39%). HPLC: t_{R}=10.0 min (97%). ¹H NMR (500 MHz, CDCl₃) δ 6.92 (dd, J=11.2, 8 Hz, 2H), 6.72 (d, J=10 Hz, 1H), 6.62-6.20 (m, 1H), 3.76 (d, J=5 Hz, 2H), 2.66-2.46 (m, 2H), 2.41-2.28 (m, 2H), 1.87-1.67 (m, 7H), 1.32-0.99 (m, 5H), 0.85 (dd, J=6.5, 4 Hz, 3H). ¹³C NMR (126 MHz, CDCl₃) δ 151.3 (d, J_{C-F}=243.2 Hz), 147.1 (d, J_{C-F}=45.0 Hz). 137.3 (d, J_{C-F}=3.8 Hz). 121.2 (d, J_{C-F}=6.3 Hz), 115.8, 115.7 (d, J_{C-F}=33.9 Hz), 75.0, 56.3 (d, J_{C-F}=2.5 Hz), 41.4 (d, J_{C-F}=6.3 Hz), 37.9, 35.7, 30.0, 26.7, 26.0, 18.1. ¹⁹F NMR (471 MHz, CDCl₃) S-138.92. HRMS (ES): (m/z) calculated for C₇H₂₆FNO [M]⁺ 279.1998; found 279.1996.

1-(3-(Cyclohexylmethoxy)phenyl)-3-(methylamino)propan-1-ol (16e). A solution of 1-(3-(cyclohexylmethoxy)phenyl)propane-1,3-diol (117 mg, 0.34 mmol) and methylamine (2.5 mL, 40% in water) in THF (2.5 mL) was heated at 70° C. for 4 h in a pressurized tube. After cooling, the solution was diluted with ether, washed with saturated aqueous sodium bicarbonate and brine, dried with anhydrous potassium carbonate, and concentrated to dryness. Purification by flash chromatography gave a colorless oil (80 mg, 85%). HPLC: t_{R}=10.8 min (95%). ¹H NMR (400 MHz, CDCl₃) δ 7.21 (t, J=8 Hz, 1H), 6.92-6.96 (m, 1H), 6.87-6.92 (m, 1H), 6.76 (dd, J=2.4, 8 Hz, 1H), 4.88 (dd, J=3.2, 8.4 Hz, 1H), 3.75 (d, J=6.4 Hz, 2H), 3.46-3.70 (bs, 2H), 2.77-2.91 (m, 2H), 2.42 (s, 3H), 1.82-1.91 (m, 3H), 1.65-1.82 (m, 5H), 1.12-1.35 (m, 3H), 0.98-1.11 (m, 2H); ¹³C NMR (100 MHz, CDCl₃) δ 159.5, 146.9, 129.2, 117.7, 113.1, 111.7, 75.4, 73.5, 50.4, 37.8, 36.9, 36.1, 30.0, 26.6, 25.9. HRMS (ES): (m/z) calculated for C₇H₂₈NO₂ [M]⁺ 277.2042; found 277.2051.

### Synthesis of Intermediates

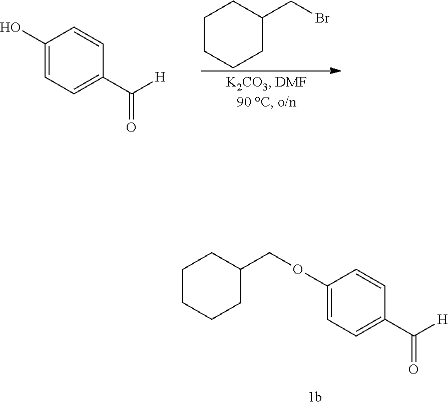 [SMILES: O=Cc1ccc(O)cc1 ; BrCC1CCCCC1 ; O=Cc1ccc(OCC2CCCCC2)cc1]

4-(cyclohexylmethoxy)benzaldehyde (1b). The solution of 4-hydroxybenzaldehyde (2.50 g, 20.5 mmol), potassium carbonate (5.8 g, 42.0 mmol), (bromomethyl)cyclohexane (3.5 mL, 25.1 mmol) and DMF (40 mL) was stirred overnight at 90° C. under argon. After allowing time to cool, the reaction mixture was diluted with water (100 mL) and extracted with ethyl acetate (3×30 mL). The combined organic layers were washed with water (100 mL), brine (100 mL), dried over anhydrous MgSO₄, filtered, and concentrated under reduced pressure. The crude oil was purified by flash column chromatography to give a yellow syrup (3.5 g, 78%). ¹H NMR (500 MHz, CDCl₃) δ 9.83 (s, ¹H), 7.82 (d, J=8.7 Hz, 2H), 6.98 (d, J=8.7 Hz, 2H) 3.83 (d, J=6.2 Hz, 2H), 1.88-1.70 (m, 6H), 1.35-1.15 (m, 3H), 1.10-1.03 (m, 2H). ¹³C NMR (126 MHz, CDCl₃) δ 190.9, 164.5, 132.1, 129.8, 114.8, 73.9, 37.6, 29.9, 26.5, 25.8. HRMS (ESI⁺): (m/z) calculated for C₁₄H₁₈O₂Na [M+Na]⁺ 241.1190; found 241.1187.

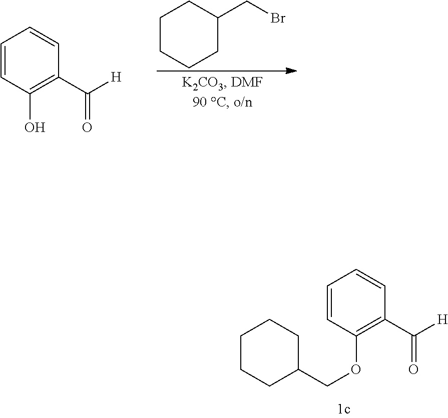 [SMILES: O=Cc1ccccc1O ; BrCC1CCCCC1 ; O=Cc1ccccc1OCC1CCCCC1]

2-(cyclohexylmethoxy)benzaldehyde (1c). The solution of 2-hydroxybenzaldehyde (2.2 mL, 20.6 mmol), potassium carbonate (5.8 g, 42.0 mmol), (bromomethyl)cyclohexane (3.5 mL, 25.1 mmol) and DMF (40 mL) was stirred overnight at 90° C. under argon. After allowing time to cool, the reaction mixture was diluted with water (100 mL) and extracted with ethyl acetate (3×30 mL). The combined organic layers were washed with water (100 mL), brine (100 mL), dried over anhydrous MgSO4, filtered, and concentrated under reduced pressure. The crude oil was purified by flash column chromatography to give a yellow syrup (4.3 g, 92%). ¹H NMR (500 MHz, CDCl₃) δ 10.54 (s, 1H), 7.83 (dd, J=7.7, 1.8 Hz, 1H), 7.52 (ddd, J=8.8, 7.3, 1.8 Hz, 1H), 7.00 (t, J=7.5 Hz, 1H), 6.97 (d, J=8.4 Hz, 1H), 3.87 (d, J=5.8 Hz, 2H), 1.91-1.84 (m, 3H), 1.82-1.68 (m, 3H), 1.37-1.18 (m, 4H), 1.16-1.06 (m, 2H). ¹³C NMR (126 MHz, CDCl₃) δ 190.0, 161.8, 136.0, 128.3, 125.1, 120.5, 112.6, 73.9, 37.8, 29.9, 26.5, 25.9. HRMS (ESI⁺): (m/z) calculated for C₁₄H₁₈O₂Na [M+Na]⁺241.1190; found 241.1198.

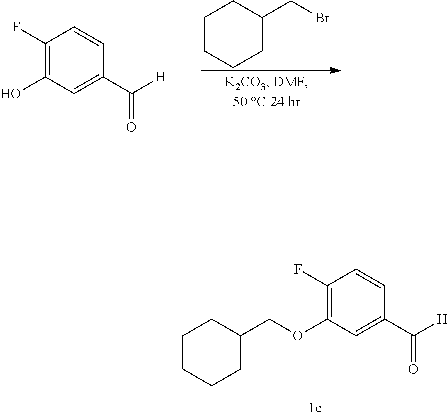 [SMILES: O=Cc1ccc(F)c(O)c1 ; BrCC1CCCCC1 ; O=Cc1ccc(F)c(OCC2CCCCC2)c1]

3-(cyclohexylmethoxy)-4-fluorobenzaldehyde (1e) 4-Fluoro-3-hydroxybenzaldehyde (1000 mg, 7.14 mmol), potassium carbonate (1568 mg, 11.36 mmol), and DMF (15 mL) were added to a 100 mL round bottom flask. Then (bromomethyl)cyclohexane (0.78 mL, 5.68 mmol) was added. The reaction was sealed by a septum and ran for 24 h at 50° C. After cooled to room temperature, the reaction mixture was poured to deionized water (300 mL) and extracted by EtOAc (3×100 mL). The combined organic layer was washed with deionized water (3×100 mL) and dried over Na₂SO₄. Solvents removed under reduced pressure and crude product was purified by flash column chromatography giving a white solid (782 mg, yield 58%). ¹H NMR (500 MHz, CDCl₃) δ 9.86 (s, 1H), 7.45 (dd, J=8.0, 2.0 Hz, 1H), 7.38 (ddd, J=8.2, 4.5, 2.0 Hz, 1H), 7.18 (dd, J=10.5, 8.0 Hz, 1H), 3.84 (d, J=6.0 Hz, 2H), 1.87-1.65 (m, 6H), 1.32-1.00 (m, 5H). ¹³C NMR (126 MHz, CDCl₃) δ 190.8, 157.7, 155.7, 148.4, 148.4, 133.2, 125.0, 124.9, 116.5, 116.4, 112.9, 112.9, 74.7, 37.5, 29.7, 26.4, 25.7. ¹⁹F NMR (471 MHz, CDCl₃) S-123.2. HRMS (ES): (m/z) calculated for C₁₄H₁₇FO₂ [M]⁺ 2326.1213; found 2326.1212.

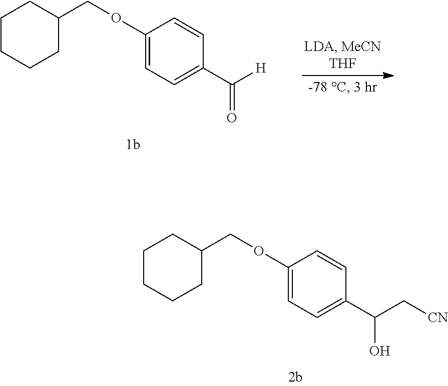 [SMILES: O=Cc1ccc(OCC2CCCCC2)cc1 ; N#CCC(O)c1ccc(OCC2CCCCC2)cc1]

3-(4-(cyclohexylmethoxy)phenyl)3-hydroxypropanenitrile (2b): nBuLi (1.6 M in hexanes, 8.0 mL, 12.8 mmol) was added dropwise to a stirred solution of diisopropylamine (1.6 mL, 11.4 mmol) and THF (30 mL) at −78° C. under argon. After stirring for 0.5 hr. acetonitrile (0.60 mL, 11.5 mmol) was added and stirred at −78° C. for 1 hr. 4-(cyclohexylmethoxy)benzaldehyde (1.5 g, 6.8 mmol) in THF (10 mL) was added to the round bottom flask in a dropwise manner then the resulting reaction mixture then was allowed to stir at −78° C. for 3 hr. The reaction mixture was allowed to warm to rt, quenched with sat. ammonium chloride (100 mL), and extracted with ethyl acetate (3×30 mL). The combined organic extracts were then washed with water (100 mL), brine (100 mL), dried over magnesium sulfate, filtered, and concentrated in vacuo. The crude oil was purified by flash column chromatography to afford a yellow solid (1.13 g, 63%). ¹H NMR (500 MHz, CDCl₃) δ 7.31 (d, J=8.7 Hz, 2H), 6.90 (d, J=8.7 Hz, 2H), 5.00 (td, J=6.3, 3.0 Hz, 1H), 3.75 (d, J=6.3 Hz, 2H), 2.82-2.70 (m, 2H), 2.16 (s, 1H), 1.90-1.66 (m, 7H), 1.37-1.15 (m, 4H), 1.05 (qd, J=12.3, 3.5 Hz, 2H). ¹³C NMR (126 MHz, CDCl₃) δ 159.78, 132.78, 126.82, 117.32, 114.87, 73.60, 69.97, 37.64, 29.88, 27.89, 26.50, 25.78. HRMS (ESI⁺): (m/z) calculated for C₁₆H₂₁NaNO₂ [M+Na]⁺ 282.1465; found 282.1465.

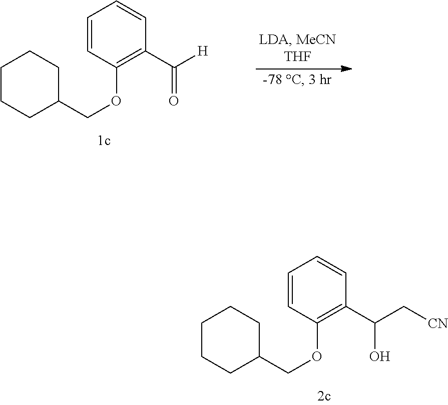 [SMILES: O=Cc1ccccc1OCC1CCCCC1 ; N#CCC(O)c1ccccc1OCC1CCCCC1]

3-(2-(cyclohexylmethoxy)phenyl)3-hydroxypropanenitrile (2c). nBuLi (1.6 M in hexanes, 8.0 mL, 12.8 mmol) was added dropwise to a stirred solution of diisopropylamine (1.6 mL, 11.4 mmol) and THF (30 mL) at −78° C. under argon. After stirring for 0.5 hr, acetonitrile (0.60 mL, 11.5 mmol) was added and stirred at −78° C. for 1 hr. 2-(cyclohexylmethoxy)benzaldehyde (1.5 g, 6.8 mmol) in THF (10 mL) was added to the round bottom flask in a dropwise manner then the resulting reaction mixture then was allowed to stir at −78° C. for 3 hr. The reaction mixture was allowed to warm to rt, quenched with sat. ammonium chloride (100 mL), and extracted with ethyl acetate (3×30 mL). The combined organic extracts were then washed with water (100 mL), brine (100 mL), dried over magnesium sulfate, filtered, and concentrated in vacuo. The crude oil was purified by flash column chromatography to afford a yellow syrup (1.0 g, 59%). ¹H NMR (500 MHz, CDCl₃) δ 7.39 (dd, J=7.5, 1.7 Hz, 1H), 7.29 (td, J=7.8, 1.7 Hz, 1H), 6.99 (td, J=7.6, 1.1 Hz, 1H), 6.89 (dd, J=8.3, 1.1 Hz, 1H), 5.22 (td, J=6.9, 5.1 Hz, 1H), 3.83 (qd, J=9.0, 5.8 Hz, 2H), 2.94 (d, J=6.4 Hz, 1H), 2.90 (dd, J=16.7, 5.1 Hz, 1H), 2.81 (dd, J=16.6, 7.4 Hz, 1H), 1.88-1.69 (m, 7H), 1.37-1.18 (m, 4H), 1.14-1.04 (m, 2H). ¹³C NMR (126 MHz, CDCl₃) δ 155.7, 129.7, 128.6, 126.8, 121.0, 117.8, 111.4, 73.6, 67.5, 37.7, 30.1, 26.5, 26.3, 25.8. HRMS (ESI⁺): (m/z) calculated for C₁₆H₂₁NaNO₂ [M+Na]⁺ 282.1465; found 282.1465.

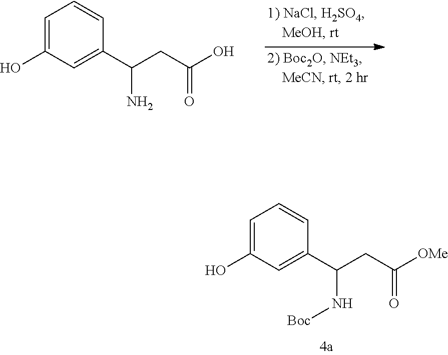 [SMILES: NC(CC(=O)O)c1cccc(O)c1 ; COC(=O)CC(NC(=O)OC(C)(C)C)c1cccc(O)c1]

Methyl 3-(tert-butyloxycarbonyl)amino)-3-(3-hydroxylphenyl)propanoate (4a). HCl gas (generated in situ by sulfuric acid addition to NaCl) was bubbled through a suspension of 3-amino-3-(3-hydroxyphenyl)propanoic acid (3.65 g, 20.1 mmol) was in methanol (20 mL). The solid dissolved after 0.5 hr of reacting and solvents were concentrated under reduced pressure to yield a white solid (4.99 g, 107%) which was used without purification. The mixture of amine salt, di-tertbuyl dicarbonate (9.5 mL. 42.1 mmol), triethylamine (3.0 mL, 21.5 mmol), and acetonitrile (20 mL) stirred for 2 hr at rt. The reaction mixture was diluted in water (100 mL) and extracted with ethyl acetate (3×30 mL). The combined organic layers were washed with 10% Na₂SO₃ (100 mL), sat. NaHCO₃ (100 mL), brine (100 mL), and dried over sodium sulfate. The solvents were concentrated under reduced pressure, and crude material was purified by flash column chromatography to yield a white solid (4.25 g. 71%, 76% over 2 steps). ¹H NMR (500 MHz, DMSO-d₆) δ 9.34 (s, 1H), 7.41 (d, J=8.8 Hz, 1H), 7.08 (t, J=7.8 Hz, 1H), 6.72-6.67 (m, 2H), 6.61 (ddd, J=8.1, 2.4, 1.0 Hz, 1H), 4.90-4.74 (m, 1H), 3.55 (s, 3H), 2.71 (dd, J=15.5, 8.8 Hz, 1H), 2.63 (dd, J=15.4, 6.3 Hz, 1H), 1.35 (s, 9H). ¹³C NMR (126 MHz, DMSO-d₆) δ 170.7, 157.2, 154.7, 144.3, 129.2, 116.8, 113.9, 113.2, 77.8, 51.3, 50.9, 40.9, 28.2. HRMS (ESI): (m/z) calculated for C₁₆H₂±NO₅Na [M+Na]⁺ 318.1317; found 318.1312.

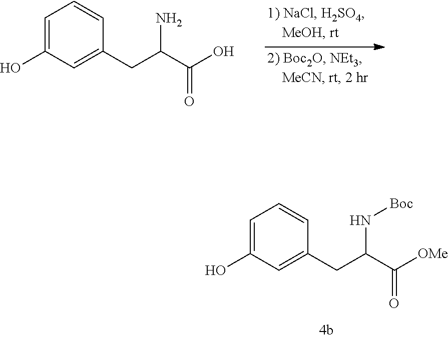 [SMILES: NC(Cc1cccc(O)c1)C(=O)O ; COC(=O)C(Cc1cccc(O)c1)NC(=O)OC(C)(C)C]

Methyl 2-(tert-butyoxycarbonyl)amino)-3-(3-hydroxylphenyl)propanoate (4b). HCl gas (generated in situ by sulfuric acid addition to NaCl) was bubbled through a suspension of 2-amino-3-(3-hydroxyphenyl)propanoic acid (600 mg, 3.3 mmol) was in methanol (5 mL). The solid dissolved after 0.5 hr of reacting and solvents were concentrated under reduced pressure to yield an amber foam (750 mg, 98%) which was used without purification. The mixture of amine salt, di-tertbuyl decarbonate (1.1 mL. 4.8 mmol), triethylamine (0.5 mL, 3.6 mmol), and acetonitrile (6 mL) stirred for 2 hr at rt. The reaction mixture was diluted in water (50 mL) and extracted with ethyl acetate (2×30 mL). The combined organic layers were washed with 10% Na₂SO₃ (50 mL), sat. NaHCO₃ (50 mL), brine (50 mL), and dried over sodium sulfate. The solvents were concentrated under reduced pressure, and crude material was purified by flash column chromatography to yield a white solid (681 mg, 76%; 74% over 2 steps). ¹H NMR (500 MHz, DMSO-d₆) δ 9.28 (s, ¹H), 7.25 (d, J=8.1 Hz, 1H), 7.05 (t, J=7.6 Hz, 1H), 6.67-6.56 (m, 3H), 4.11 (td, J=8.7, 8.2, 5.2 Hz, 1H), 3.60 (s, 3H), 2.88 (dd, J=13.8, 5.1 Hz, 1H), 2.75 (dd, J=13.7, 9.9 Hz, 1H), 1.33 (s, 9H). ¹³C NMR (126 MHz, DMSO-d₆) δ 172.6, 157.2, 155.4, 138.9, 129.1, 119.6, 115.9, 113.4, 78.2, 55.2, 51.7, 36.4, 28.1. HRMS (ESI): (m/z) calculated for C₁₆H₂₆NO₅Na [M+Na]⁺ 318.1317; found 318.1311.

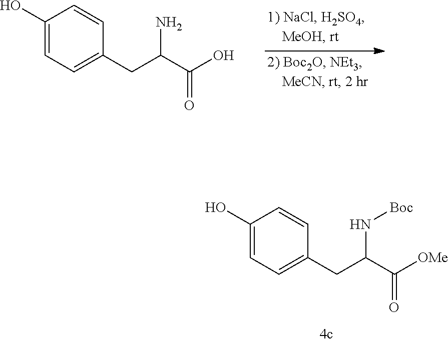 [SMILES: NC(Cc1ccc(O)cc1)C(=O)O ; COC(=O)C(Cc1ccc(O)cc1)NC(=O)OC(C)(C)C]

Methyl 2-(tert-butyoxycarbonyl)amino)-3-(4-hydroxylphenyl)propanoate (4c). HCl gas (generated in situ by sulfuric acid addition to NaCl) was bubbled through a suspension of L-tyrosine (3.65 g, 20.1 mmol) was in methanol (20 mL). The solid dissolved after 0.5 hr of reacting and solvents were concentrated under reduced pressure to yield a brown syrup (5.54 g) which was used without purification. The mixture of syrup, di-tertbuyl dicarbonate (9.5 mL. 41.4 mmol), triethylamine (3.0 mL, 21.5 mmol), and acetonitrile (20 mL) stirred for 2 hr at rt. The reaction mixture was diluted in water (100 mL) and extracted with ethyl acetate (3×30 mL). The combined organic layers were washed with 10% Na₂SO₃ (100 mL), sat. NaHCO₃ (100 mL), brine (100 mL), and dried over sodium sulfate. The solvents were concentrated under reduced pressure, and crude material was purified by flash column chromatography to yield a colorless syrup (4.50 g, 75%, 89 over 2 steps). ¹H NMR (500 MHz, CDCl₃) δ 9.21 (s, 1H), 7.22 (d. J=8.0 Hz. 1H), 7.00 (d, J=8.0 Hz, 2H), 6.65 (d, J=8.4 Hz, 2H), 4.11-4.01 (m, 1H), 2.84 (dd, J=13.9, 5.3 Hz, 1H), 2.72 (dd, J=13.4, 8.8 Hz, 1H), 1.33 (s, 9H). ¹³C NMR (126 MHz, CDCl₃) δ 172.7, 155.9, 155.4, 129.9, 127.5, 114.9, 78.2, 55.6, 51.7, 35.7, 28.1.

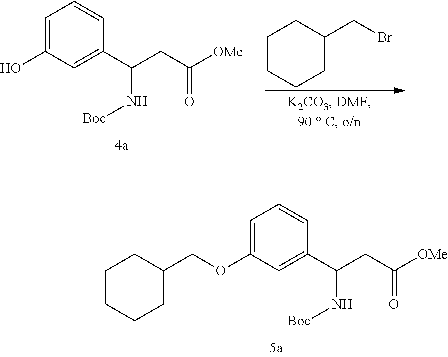 [SMILES: COC(=O)CC(NC(=O)OC(C)(C)C)c1cccc(O)c1 ; BrCC1CCCCC1 ; COC(=O)CC(NC(=O)OC(C)(C)C)c1cccc(OCC2CCCCC2)c1]

Methyl 3-((tert-butoxycarbonyl-3-(3-cyclohexylmethoxy)phenyl)propanoate (5a). The solution of compound 4a (800 mg, 2.7 mmol), potassium carbonate (500 mg, 3.6 mmol), (bromomethyl)cyclohexane (0.45 mL, 3.2 mmol) and DMF (10 mL) was stirred overnight at 90° C. under argon. After allowing time to cool, the reaction mixture was diluted with water (30 mL) and extracted with ethyl acetate (3×31 mL). The combined organic layers were washed with water (30 mL), brine (30 mL), dried over anhydrous Na₂SO₄, filtered, and concentrated under reduced pressure. The crude oil was purified by flash column chromatography to give a colorless syrup (426 mg, 40%). ¹H NMR (500 MHz, DMSO-d₆) δ 7.43 (d, J=8.9 Hz, 1H), 7.19 (t, J=7.9 Hz, 1H), 6.87 (s, 1H), 6.83 (d, J=7.6 Hz, 1H), 6.77 (dd, J=8.2, 2.5 Hz, 1H), 4.93-4.83 (m, 1H), 3.75 (d, J=6.4 Hz, 2H), 3.56 (s, 3H), 2.88-2.77 (m, 1H), 2.72 (dd, J=15.4, 8.8 Hz, 1H), 2.66 (dd, J=15.4, 6.2 Hz, 1H), 1.84-1.77 (m, 2H), 1.76-1.62 (m, 5H), 1.36 (s, 9H), 1.30-1.20 (m, 4H), 1.04 (qd, J=12.3, 3.2 Hz, 2H). HRMS (EI): (m/z) calculated for C₂₂H₃₃NO₅ [M]⁺ 391.2359; found 391.2358.

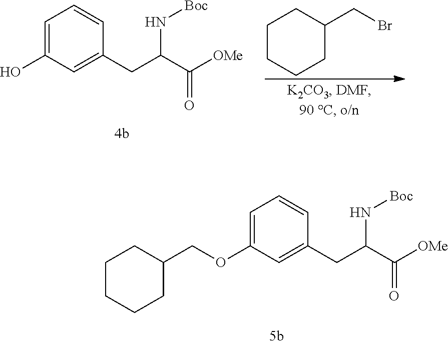 [SMILES: COC(=O)C(Cc1cccc(O)c1)NC(=O)OC(C)(C)C ; BrCC1CCCCC1 ; COC(=O)C(Cc1cccc(OCC2CCCCC2)c1)NC(=O)OC(C)(C)C]

Methyl 2-((tert-butoxycarbonyl)-3-(3-cyclohexylmethoxy)phenyl)propanoate (5b). The solution of compound 4b (1.12 g, 3.8 mmol), potassium carbonate (600 mg, 4.3 mmol), (bromomethyl)cyclohexane (0.60 mL, 4.3 mmol) and DMF (10 mL) was stirred overnight at 90° C. under argon. After allowing time to cool, the reaction mixture was diluted with water (50 mL) and extracted with ethyl acetate (3×30 mL). The combined organic layers were washed with water (100 mL), brine (100 mL), dried over anhydrous Na₂SO₄, filtered, and concentrated under reduced pressure. The crude oil was purified by flash column chromatography to give a white solid (656 mg, 44%). ¹H NMR (500 MHz, DMSO-d₆) δ 7.24 (d, J=8.1 Hz, 1H), 7.11 (d, J=8.5 Hz, 2H), 6.81 (d, J=8.6 Hz, 2H), 4.09 (ddd, J=9.9, 8.0, 5.2 Hz, 1H), 3.72 (d, J=6.4 Hz, 2H), 3.60 (s, 3H), 2.90 (dd, J=13.9, 5.2 Hz, 1H), 2.76 (dd, J=13.8, 9.9 Hz, 1H), 1.82-1.60 (m, 6H), 1.32 (s, 8H), 1.28-1.13 (m, 5H), 1.07-0.96 (m, 2H). ¹³C NMR (126 MHz, DMSO-d₆) δ 172.7, 157.5, 155.4, 130.0, 129.2, 114.1, 78.2, 72.6, 55.5, 51.7, 37.0, 35.6, 29.2, 28.1, 26.0, 25.2. HRMS (ESI): (m/z) calculated for C₂₂H₃₃KNO₅ [M+K]⁺ 430.1996; found 430.1991.

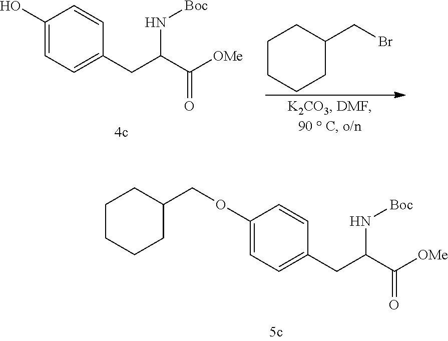 [SMILES: COC(=O)C(Cc1ccc(O)cc1)NC(=O)OC(C)(C)C ; BrCC1CCCCC1 ; COC(=O)C(Cc1ccc(OCC2CCCCC2)cc1)NC(=O)OC(C)(C)C]

Methyl 2-((tert-butoxycarbonyl)-3-(4-cyclohexylmethoxy)phenyl)propanoate (5c). The solution of compound 4c (1.12 g, 3.8 mmol), potassium carbonate (600 mg, 4.3 mmol), (bromomethyl)cyclohexane (0.60 mL, 4.3 mmol) and DMF (10 mL) was stirred overnight at 90° C. under argon. After allowing time to cool, the reaction mixture was diluted with water (50 mL) and extracted with ethyl acetate (3×30 mL). The combined organic layers were washed with water (100 mL), brine (100 mL), dried over anhydrous Na₂SO₄, filtered, and concentrated under reduced pressure. The crude oil was purified by flash column chromatography to give a white solid (656 mg, 44%). ¹H NMR (500 MHz, DMSO-d₆) δ 7.24 (d, J=8.1 Hz, 1H), 7.11 (d, J=8.5 Hz, 2H), 6.81 (d, J=8.6 Hz, 2H), 4.09 (ddd, J=9.9, 8.0, 5.2 Hz, 1H), 3.72 (d, J=6.4 Hz, 2H), 3.60 (s, 3H), 2.90 (dd, J=13.9, 5.2 Hz, 1H), 2.76 (dd, J=13.8, 9.9 Hz, 1H), 1.82-1.60 (m, 6H), 1.32 (s, 8H), 1.28-1.13 (m, 5H), 1.07-0.96 (m, 2H). ¹³C NMR (126 MHz, DMSO-d₆) δ 172.7, 157.5, 155.4, 130.0, 129.2, 114.1, 78.2, 72.6, 55.5, 51.7, 37.0, 35.6, 29.2, 28.1, 26.0, 25.2.

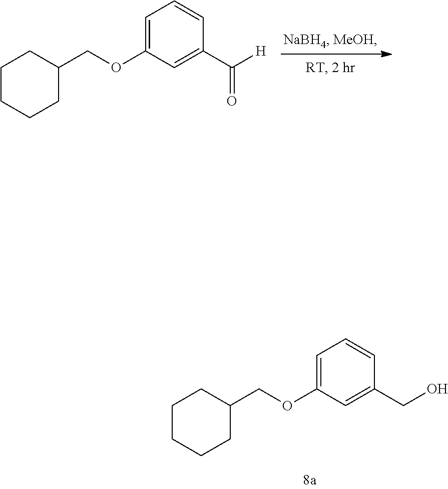 [SMILES: O=Cc1cccc(OCC2CCCCC2)c1 ; OCc1cccc(OCC2CCCCC2)c1]

(3-(cyclohexylmethoxy)phenyl)methanol (8a). 3-(cyclohexylmethoxy) benzaldehyde (1.10 g, 5.0 mmol) in THF (5 mL) was added dropwise to an ice-cold stirred solution of lithium aluminum hydride (400 mg, 10.54 mmol) in THF (10 mL) under argon. The resulting mixture stirred at 0° C. for 1 hr then was slowly quenched by the dropwise addition of water (1.1 mL), 15% NaOH (1.1 mL), and water (3.3 mL) consecutively. The reaction mixture was allowed to stir at rt for 0.5 hr, filtered through cotton, diluted with water (30 mL), and extracted with ethyl acetate (2×20 mL). All organic extracts were combined, washed with water (100 mL), brine (100 mL), dried over sodium sulfate, and filtered. Following concentration in vacuo, the crude product was then purified by flash column chromatography giving a colorless oil (705 mg, 63%). ¹H NMR (500 MHz, CDCl₃) δ 7.29-7.23 (m, 2H), 6.96-6.88 (m, 2H), 6.82 (dd, J=8.9, 2.2 Hz, 1H), 4.67 (d, J=6.0 Hz, 2H), 3.76 (d, J=6.4 Hz, 2H). 1.90-1.84 (m, 2H), 1.80-1.67 (m, 4H), 1.62 (t, J=6.1 Hz, 1H), 1.36-1.15 (m, 3H), 1.05 (qd, J=12.1, 3.4 Hz, 2H). ¹³C NMR (126 MHz, CDCl₃) δ 159.7, 142.5, 129.7, 118.9, 114.0, 113.0, 73.6, 65.5, 37.8, 30.0, 26.6, 25.9.

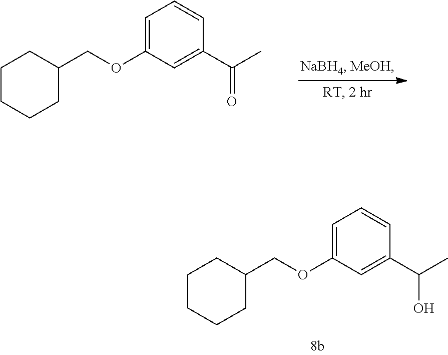 [SMILES: CC(=O)c1cccc(OCC2CCCCC2)c1 ; CC(O)c1cccc(OCC2CCCCC2)c1]

1-(3-(cyclohexylmethoxy)phenyl)ethan-1-ol (8b). Sodium borohydride (1.4 g, 37.0 mmol) was added in portions to a stirred solution of 1-(3-(cyclohexylmethoxy)phenyl)ethan-1-one (4.20 g, 18.0 mmol) in MeOH (30 mL). The resulting mixture stirred at RT for 2 hr then was slowly quenched by the dropwise addition of water (100 mL) and extracted with ethyl acetate (3×30 mL). All organic extracts were combined, washed with brine (100 mL), dried over sodium sulfate, and filtered. Following concentration in vacuo, the crude product was then purified by flash column chromatography giving an amber syrup (3.13 g, 74%). ¹H NMR (500 MHz, CDCl₃) δ 7.24 (s, 1H), 6.92 (d, J=10.9 Hz, 2H), 6.80 (d, J=7.5 Hz, 1H), 4.87 (q, J=6.9 Hz, 1H), 3.76 (d, J=6.4 Hz, 2H), 1.92-1.66 (m, 7H), 1.49 (d, J=6.5 Hz, 3H), 1.27 (d, J=73.6 Hz, 3H), 1.05 (qd, J=12.3, 3.2 Hz, 2H). ¹³C NMR (126 MHz, CDCl₃) δ 159.7, 147.6, 129.6, 117.5, 113.5, 111.6, 73.6, 70.5, 37.8, 30.0, 26.6, 25.9, 25.2.

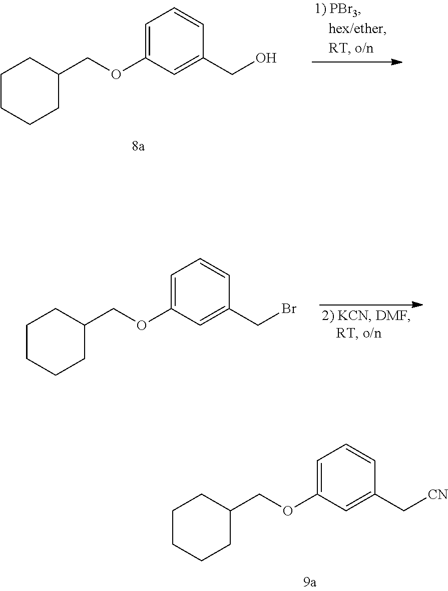 [SMILES: OCc1cccc(OCC2CCCCC2)c1 ; BrCc1cccc(OCC2CCCCC2)c1 ; N#CCc1cccc(OCC2CCCCC2)c1]

2-(3-(cyclohexylmethoxy)phenyl)acetonitrile (9a). Phosphorus tribromide (100 μL, 1.0 mmol) in hexanes/ether mixture (1:1, 2 mL) was added dropwise to a solution of alcohol 8a (650 mg, 2.95 mmol) in hexanes/ether (1:1, 8 mL) at −30° C. The resulting mixture then warmed to rt and stir overnight. The reaction mixture was quenched with sat. bicarbonate (30 mL), extracted with ethyl acetate (3×20 mL) and all organic layers were combined. The organic extracts were washed with water (50 mL), brine (50 mL), dried over sodium sulfate, and filtered. Solvents were removed in-vacuo giving a benzylic bromide as a colorless oil (671 mg, 80%) which was used without purification. ¹H NMR (500 MHz, CDCl₃) δ 7.23 (t, J=7.9 Hz, 1H), 6.95 (d, J=7.8 Hz, 1H), 6.92 (s, 1H), 6.82 (dd, J=8.2, 2.5 Hz, 1H), 4.46 (s, 2H), 3.75 (d, J=6.3 Hz, 2H), 1.90-1.83 (m, 2H), 1.80-1.66 (m, 4H), 1.36-1.17 (m. 3H), 1.05 (qd, J=12.2, 3.5 Hz, 2H). ¹³C NMR (126 MHz, CDCl₃) δ 159.6, 139.2, 129.9, 121.1, 115.1, 114.8, 73.6, 37.8, 33.7, 30.0, 26.6, 25.9.

The aforementioned synthesized bromide (570 mg, 2.0 mmol) was dissolved in DMF (15 mL) along with potassium cyanide (400 mg, 6.1 mmol) and allowed to stir overnight at RT. The solution was diluted in water (30 mL) and extracted with EtOAc (2×20 mL) and organic extracts were combined and consecutively washed with water (50 mL), brine (50 mL), and dried over sodium sulfate. Solvents were removed in-vacuo and crude material was purified by flash column chromatography to give a yellow syrup (320 mg, 69%). ¹H NMR (500 MHz, CDCl₃) δ 7.29-7.24 (m, 1H), 6.90-6.82 (m, 3H), 3.75 (d, J=6.4 Hz, 2H), 3.71 (s, 2H), 1.90-1.83 (m, 2H), 1.82-1.66 (m, 4H), 1.36-1.15 (m, 3H), 1.05 (qd, J=12.2, 3.4 Hz, 2H). ¹³C NMR (126 MHz, CDCl₃) δ 160.0, 131.3, 130.2, 119.9, 117.9, 114.3, 114.2, 73.7, 37.8, 30.0, 26.6, 25.9, 23.7. HRMS (EI): (m/z) calculated for C₁₅H₁₉NO [M]⁺ 229.1467; found 229.1472.

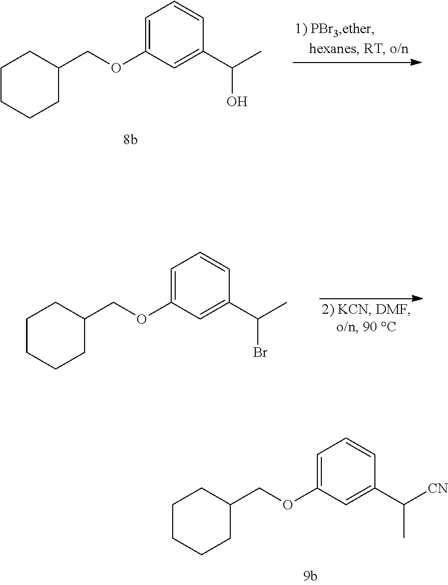 [SMILES: CC(O)c1cccc(OCC2CCCCC2)c1 ; CC(Br)c1cccc(OCC2CCCCC2)c1 ; CC(C#N)c1cccc(OCC2CCCCC2)c1]

2-(3-(cyclohexylmethoxy)phenyl)propanenitrile (9b). Phosphorus tribromide (0.55 mL, 5.8 mmol) in hexane/ether (1:1, 10 mL) was added dropwise to a solution of the alcohol 8b (3.0 g, 12.8 mmol) in hexane/ether (1:1, 30 ml). The reaction mixture was stirred overnight at RT then carefully quenched with sat. aq. NaHCO₃ (100 mL) was added dropwise until no bubbling was observed. The solution was extracted with ethyl acetate (2×30 ml) and the combined extracts washed with water (100 ml) and brine (100 ml), dried and concentrated under reduced pressure to obtain a light amber syrup (3.39 g, 89%) which was used without further purification. ¹H NMR (500 MHz, CDCl₃) δ 7.23 (t, J=8.0 Hz, 1H), 6.99 (d, J=7.8 Hz, 2H), 6.81 (d, J=8.2 Hz, 1H), 5.17 (q, J=7.0 Hz, 1H), 3.75 (d, J=6.4 Hz, 2H), 2.03 (d, J=6.9 Hz, 3H), 1.88 (d, J=13.0 Hz, 2H), 1.74 (d, J=42.4 Hz, 4H), 1.27 (d, J=73.3 Hz, 3H), 1.06 (qd, J=12.1, 3.3 Hz, 2H). ¹³C NMR (126 MHz, CDCl₃) δ 159.5, 144.7, 129.7, 118.9, 114.4, 113.2, 73.6, 49.7, 37.8, 30.0, 26.9, 26.6, 25.9.

Synthetized benzylic bromide (3.39 g, 11.4 mmol), potassium cyanide (2.25 g, 34.5 mmol) in DMF (30 mL) was stirred at 90° C. for 24 hrs. The resulting mixture was cooled to RT then diluted with water (100 mL) and extracted with ethyl acetate (3×20 mL). All organic extracts were combined, washed with water (50 mL), brine (50 mL), dried over sodium sulfate, and filtered. Following concentration in vacuo, the crude product was then purified by flash column chromatography to give a yellow syrup (628 mg, 22%). ¹H NMR (500 MHz, CDCl₃) δ 7.28 (d, J=8.1 Hz, 2H), 6.93-6.87 (m, 2H), 6.84 (d, J=8.4 Hz, 1H), 3.86 (q, J=7.3 Hz, 1H), 3.76 (d, J=6.3 Hz, 2H), 1.87 (d, J=13.2 Hz, 2H), 1.74 (d, J=42.3 Hz, 4H), 1.64 (d, J=7.3 Hz, 3H), 1.27 (d, J=73.2 Hz, 3H), 1.06 (qd, J=12.3, 3.3 Hz, 2H). ¹³C NMR (126 MHz, CDCl₃) δ 160.0, 138.5, 130.2, 121.7, 118.8, 114.0, 113.2, 73.7, 37.8, 31.4, 30.0, 26.6, 25.9, 21.5. HRMS (EI): (m/z) calculated for C₆H₂₁NO [M]⁺ 243.1623; found 243.1619.

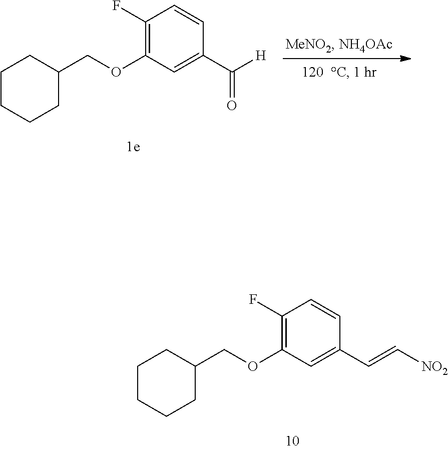 [SMILES: O=Cc1ccc(F)c(OCC2CCCCC2)c1 ; O=[N+]([O-])/C=C/c1ccc(F)c(OCC2CCCCC2)c1]

(E)-2-(cyclohexylmethoxy)-1-fluoro-4-(2-nitrovinyl)benzene (10). Aldehyde 1e (782 mg, 3.31 mmol), ammonium acetate (332 mg, 4.31 mmol), and nitromethane (3.54 mL, 66.2 mmol) were added to a 50 mL sealed tube react 1 h at 120° C. with stirring. After cooled to room temperature, solvent was removed under reduced pressure and crude product was purified by flash column chromatography giving a white solid (414 mg, yield 45%). ¹H NMR (500 MHz, CDCl₃) δ 7.92 (d, J=15.0 Hz, 1H), 7.50 (d, J=15.0 Hz, 1H), 7.13=7.06 (m, 3H), 3.83 (d, J=6.5 Hz, 2H), 1.88-1.68 (m, 6H), 1.34-1.02 (m, 5H). ¹³C NMR (126 MHz, CDCl₃) δ 156.1, 154.1, 148.3, 148.2, 138.4, 136.8, 136.7, 126.6, 126.5, 122.9, 122.8, 117.2, 117.0, 114.4, 114.3, 74.9, 37.6, 29.7, 26.4, 25.7. ¹⁹F NMR (471 MHz, CDCl₃) δ-126.5. HRMS (ES): (m/z) calculated for C₁₅H₁₈FNO₃ [M]⁺ 279.1271; found 279.1270.

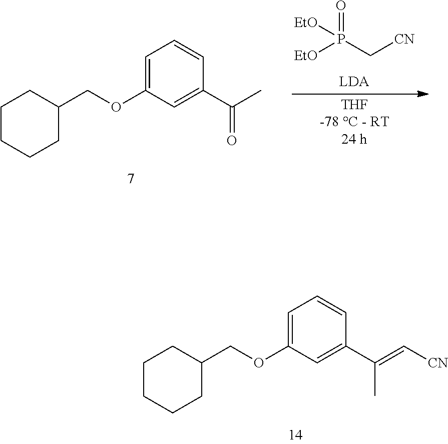 [SMILES: CC(=O)c1cccc(OCC2CCCCC2)c1 ; CCOP(=O)(CC#N)OCC ; C/C(=C\C#N)c1cccc(OCC2CCCCC2)c1]

3-(3-(cyclohexylmethoxy)phenyl)but-2-enenitrile (14) (trans:cis=10:1). Diethyl (cyanomethyl)phosphonate (1.13 mL, 7 mmol) and THF (10 mL) were added to a 100 mL round bottom flask. The flask was sealed with a septum and transferred to −78° C. followed by injecting lithium diisopropylamide (2M in THF, 3.5 mL, 7 mmol). The reaction was ran for 1 h under −78° C. with stirring, then acetophenone 7 (810 mg, 3.5 mmol) in THF (10 mL) was injected. The reaction was ran for another 23 h by slowly warming up to room temperature. After the reaction is complete, the crude reaction mixture was concentrated under reduced pressure and purified by flash column chromatography giving a white solid (818 mg, 92%). ¹H NMR (500 MHz, CDCl₃) δ7.27 (t, J=8.0 Hz, 1H), 6.99 (d, J=8.0 Hz, 1H), 6.94-6.91 (m, 2H), 5.58+5.35 (s, 1H), 3.74 (d, J=6.5 Hz, 2H), 2.42+2.24 (s, 3H), 1.87-1.67 (m, 6H), 1.33-1.00 (m, 5H). ¹³C NMR (126 MHz, CDCl₃) δ 159.8, 159.6, 139.6, 139.1, 129.82, 129.7, 119.2, 118.0, 117.6, 117.5, 116.1, 115.9, 113.2, 112.5, 95.7, 95.5, 73.6, 73.6, 37.7, 37.7, 29.9, 26.5, 25.8, 24.7, 20.3. HRMS (ES): (m/z) calculated for C₁₇H₂₁NO [M]+255.1623; found 255.1622.

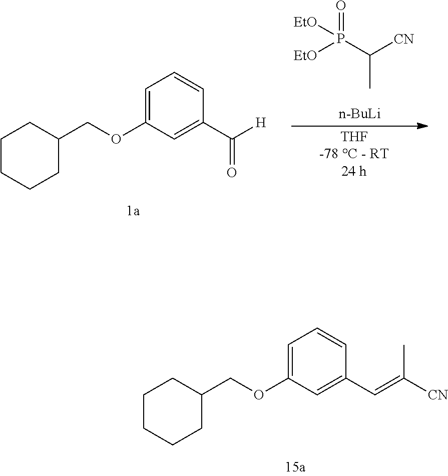

3-(3-(cyclohexylmethoxy)phenyl)-2-methylacrylonitrile (15a) (trans:cis=2.5:1). Diethyl (1-cyanoethyl)phosphonate (0.76 mL, 4.3 mmol) and THF (10 mL) were added to a 100 mL round bottom flask. The flask was sealed with a septum and transferred to −78° C. followed by injecting n-butyllithium (2.5 M in THF, 1.73 mL, 4.3 mmol). The reaction was ran for 1 h under −78° C. with stirring, then compound 1a (470 mg, 2.2 mmol) in THF (10 mL) was injected. The reaction was ran for another 23 h by slowly warming up to room temperature. After the reaction is complete, the crude reaction mixture was concentrated under reduced pressure and purified by flash column chromatography giving a pale yellow oil (458 mg, yield 83%). ¹H NMR (500 MHz, CDCl₃) δ 7.37 (t, J=7.8 Hz, 1H), 7.34-7.29 (m, 1H), 6.99-6.95 (m, 2H), 6.90 (s, 1H), 3.85+3.82 (d, J=6.5 Hz, 2H), 2.22+2.21 (s, 3H), 1.95-1.76 (m, 6H), 1.42-1.09 (m. 5H). ¹³C NMR (126 MHz, CDCl₃) δ 159.7, 159.6, 144.6, 144.2, 135.5, 135.2, 129.9, 129.9, 121.6, 121.4, 121.0, 119.4, 116.8, 115.6, 115.5, 113.9, 110.0, 106.3, 73.8, 73.7, 37.8, 37.8, 30.0, 30.0, 26.7, 26.7, 26.0, 26.0, 22.4, 17.1. HRMS (ES): (m/z) calculated for C₁₇H₂₁NO [M]+ 255.1623; found 255.1622.

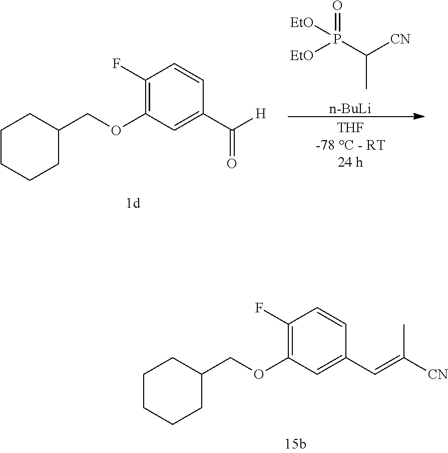

3-(3-(cyclohexylmethoxy)-4-fluorophenyl)-2-methylacrylonitrile (15b) (trans:cis=3:1). Diethyl (1-cyanoethyl)phosphonate (1.33 mL, 7.53 mmol) and THF (10 mL) were added to a 100 mL round bottom flask. The flask was sealed with a septum and transferred to −78° C. followed by injecting n-butyllithium (2.5 M in THF, 3.0 mL, 7.53 mmol). The reaction was ran for 1 h under −78° C. with stirring, then compound id (888 mg, 3.76 mmol) in THF (10 mL) was injected. The reaction was ran for another 23 h by slowly warming up to room temperature. After the reaction is complete, the crude reaction mixture was concentrated under reduced pressure and purified by flash column chromatography giving a pale yellow oil (878 mg, yield 86%). ¹H NMR (500 MHz, CDCl₃) δ 7.44-7.02 (m, 2H), 6.87-6.83 (m, 1H), 3.84+3.78 (d, J=6.5 Hz, 2H), 2.12+2.11 (s, 3H), 1.88-1.67 (m, 6H), 1.33-1.00 (m, 5H). ¹³C NMR (126 MHz, CDCl₃) δ 153.5 (d, J_{C-F}=252.0 Hz), 153.2 (d, J_{C-F}=252.0 Hz), 147.7 (d, J_{C-F}=11.3 Hz), 143.8, 143.4, 130.7 (d, J_{C-F}=3.8 Hz), 130.4 (d, J_{C-F}=3.8 Hz), 122.3 (d, J_{C-F}=7.6 Hz), 122.0 (d, J_{C-F}=7.6 Hz), 121.4, 119.5, 116.5 (d, J_{C-F}=18.9 Hz), 116.4 (d, J_{C-F}=18.9 Hz), 116.02 (d, J_{C-F}=2.5 Hz), 114.07 (d, J_{C-F}=2.5 Hz), 109.6 (d, J_{C-F}=2.5 Hz), 105.7 (d, J_{C-F}=2.5 Hz), 75.1, 74.9, 37.8, 37.7, 29.9, 29.9, 26.6, 26.6, 25.9, 25.9, 22.2. ¹⁹F NMR (471 MHz, CDCl₃) δ-130.83, -131.52. HRMS (ES): (m/z) calculated for C₁₇H₂₀FNO [M]+ 273.1529; found 273.1527.

### Transformation, Growth, and Purification of CRALBP

Plasmid (pET19b-CRALBP) was transformed by heat shock into BL-21, which was then grown in LB media at 37° C. until an OD₆₀₀ of 0.5 was obtained. CRALBP expression was then induced with the addition of IPTG (0.1 mM final concentration) followed by culturing for 14 h at 22° C. with shaking (200 rpm). Cells were pelleted by centrifugation (10 000 g at 4° C. for 15 min), resuspended in buffer, and then subjected to nitrogen cavitation (2×900 psi) with stirring. Lysed cells were centrifuged (30 000 g at 4° C. for 30 min), and the supernatant was collected. CRALBP was purified by Ni-sepharose chromatography and de-salted on a HiPrep 26/10 column. Fractions were monitored by 280 nm absorbance and collected. Protein concentration was determined by the Bradford assay.

### RPE65 Activity Assay In Vitro

Synthesized amines in DMF (1 μL) were added to a suspension of microsomal protein (20 mg), 1% bovine serum albumin, 5 mM disodium pyrophosphate, and 16.4 M apo-retinaldehyde binding protein (CRALBP) in 10 mM BTP (pH=7.4) buffer to a final concentration between 0 and 10 μM. After 2 min, all-trans retinol (5 mM in DMF, 0.5 μL) was added and the resulting mixture was incubated at 37° C. for 1 h in the dark. Under dim light, the reaction was quenched with methanol (300 L), extracted with hexanes (300 μL), and all samples were shaken violently by hand for 5 min and then stirred at 13 000 g for 5 min. The organic layer was collected and 11-cis-retinol was quantified by normal-phase HPLC using a LUNA prep silica (10 μm, 250×4.6 mm²; Phenomenex) column with an isocratic gradient of 10% (v/v) ethyl acetate in hexanes at a flow rate of 1.4 mL/min. Retinoids were detected by monitoring absorbance at 325 and 360 nm and quantified by comparison with a standard curve generated with authentic 11-cis-retinal and retinyl palmitate and area under the curve. Results were normalized by dividing the activity obtained without inhibitor. IC₅₀ and relative SD were obtained by fitting the results from each inhibitor using the [inhibitor] vs normalized response—variable slope function of GraphPad Prism software.

### Study Animals and Approvals

7-week-old BALB/cJ albino mice were purchased from the Jackson Laboratory (Jackson Laboratory; Bar Harbor; strain #000651). Equal numbers of males and females were used for experiments. All mice were housed in the vivarium at the University of California, Irvine, where they were maintained on a normal mouse chow diet and a 12 h/12 h light (<10 lux)/dark cycle. All animal procedures were approved by the Institutional Animal Care and Use Committees (IACUC) of the University of California, Irvine, and the VA Long Beach Health Care System and were conducted in accordance with the Association for Research in Vision and Ophthalmology Statement for the Use of Animals in Ophthalmic and Visual Research.

In vivo Visual Chromophore Regeneration Assays

All mice were dark-adapted for 24 h prior experiments, and all drug administration procedures were performed under dim red light. One hour before the bleaching light exposure, the animals were administered test compounds (3a, 10b, 15e, and 15b-d) by intraperitoneal (IP) injection at a dose of 10 mg/kg in 50 μL of DMSO vehicle. Vehicle-only and nontreated animals served as controls. 15 min prior to the bleaching light exposure, the mice were administered 0.5% tropicamide ophthalmic drops (Patterson Veterinary Supply, Inc.) to dilate their pupils. The mice were placed in a 2 L glass beaker contained within a ventilated white bucket and exposed to 10 000 lux white light for 10 min. Three animals per group were immediately euthanized, and their eyes were enucleated, transferred in foil-wrapped tubes, and placed on dry ice. The remaining animals were placed back in their home cages and transferred to a dark room to allow retinoid recovery to occur for either 4 or 24 h before collecting the eyes. Three animals were used for each time point.

The eyes of each animal were homogenized in 10 mM sodium phosphate buffer (pH 8.0) containing 50% methanol (vol/vol) (Sigma-Aldrich; 34860-1L-R) and 100 mM hydroxylamine (Sigma-Aldrich: 159417-100G). After 15 min incubation at room temperature, 2 mL of 3 M sodium chloride was added to the homogenate. The resulting sample was extracted twice with 3 mL of ethyl acetate (Fisher Scientific; E195-4). The combined organic phase was dried in vacuo and reconstituted in 450 μL of hexane. Retinoids extracts (100 μL) were separated on a normal-phase HPLC column (Zorbax Sil; 5 μm; 4.6 mm×250 mm; Agilent Technologies) with a stepwise gradient of 0.6% v/v ethyl acetate in hexane (Fisher Scientific; H302-4) at a flow rate of 1.4 mL/min for 17 min and 10% v/v ethyl acetate in hexane at a flow rate of 1.4 mL/min for 25 min. Retinoids were detected by monitoring absorbance at 325 and 360 nm on an Agilent 1200 HPLC system equipped with a diode array detector (DAD). Peaks were identified based on their absorbance spectra and retention times relative to authentic standards. Absolute quantification of retinoids was carried out by peak integration with reference to standard curves generated using authentic standards.

### VAP-1 Oxidation Assay

Mouse aorta homogenates were used as the source of VAP-1 for this study. Aortas were removed from mice (4-6 weeks old) that had been euthanized by CO₂ asphyxiation followed by cervical dislocation. The aortas were dissected and the blood was removed by rinsing the tissue with phosphate-buffered saline. Aorta samples were used immediately or stored at −80° C. until needed. Two aortas were minced using a stainless-steel single-edge blade and homogenized in a KONTES Potter-Elvehjem tissue grinder/homogenizer glass pestle in 1 mL of 10 mM HEPES NaOH, pH 7.6. The homogenate was collected into a 1.5 mL Eppendorf tube. 5 μL of a 20 mM ethanolic stock solution of 3a or 16e was added to the aorta homogenate to give a final substrate concentration of 100 μM. The sample was mixed and then incubated at 28° C. with 300 rpm shaking in an Eppendorf thermomixer. 200 μL samples were taken at 0, 1, and 2 h after the initiation of the reaction. At each time point, the reactions were immediately quenched with 100 μL of 100% MeOH, vortexed for 3 s, and stored at −20° C. After samples from all time points were collected and frozen, the samples were thawed and centrifuged at 15 000 rpm for 10 min; 250 μL of each supernatant was collected, placed into a borosilicate tube, and dried in a Speedvac rotary evaporator. Each dried sample was redissolved in 300 μL of a 1:1 MeOH/H₂O solution, centrifuged to remove particulates, and then transferred to an HPLC vial. 50 μL of the sample was used for analysis on an Agilent 1260 Infinity series HPLC equipped with a Proshell EC-18 column and a diode array detector. The sample was separated using a mobile phase consisting of 0.1% (v/v) formic acid in H₂O and acetonitrile at the following ratios and time intervals: 95:5 for 2 min, a gradient from 95:5 to 15:85 over 8 min, a gradient from 15:85 to 2:98 over 0.5 min, continued 2:98 for 4 min, and then a gradient from 2:98 to 95:5 over 0.5 min. The reaction substrate and product were assessed by monitoring absorbance at 275 nm. 3a and 16e were eluted at ~8.5 min, while the assay product (ACU-5201) was eluted at ~13.25 min. A dilution series of known concentrations of authentic ACU-5201 in 1:1 MeOH/H₂O was run to generate a standard curve and facilitate the conversion of product AUCs to absolute concentration.

### Liver Microsomes Assay

Mouse liver homogenates were used as the source for this study following a published protocol. Briefly, livers were removed from mice (4-6 weeks old) that had been euthanized by CO₂ asphyxiation followed by cervical dislocation. The livers were dissected, and the blood was removed by rinsing the tissue with phosphate-buffered saline. Liver samples were used immediately or stored at −80° C. until needed. Two livers were minced using a stainless-steel single-edge blade and homogenized in a KONTES Potter-Elvehjem tissue grinder/homogenizer glass pestle in 1 mL of 10 mM PBS/10 mM KCl, pH 7.4. The microsomes were subsequently stored in 0.1 M PBS/20% glycerol, pH 7.4. Assay: to a tube containing 415.6 μL of 0.1 M PBS buffer at pH 7.4 prewarmed at 37° C., 50 μL of NADPH 10 mM, and the appropriate volumes of a 10 mM ethanolic stock solution of 3a, 16e, or atomoxetine to achieve a final concentration of 15-200 μM in 500 μL was added 30 μL of liver homogenate at the concentration of 8.5 mg of protein/mL to achieve a final protein concentration of 0.5 mg/mL. The sample was mixed and then incubated at 37° C. with 300 rpm shaking in an Eppendorf thermomixer. 100 μL samples were taken at 0, 5, 15, 30, and 60 min after the initiation of the reaction. At each time point, the reactions were immediately quenched with 100 μL of 100% MeOH, vortexed for 3 s, and stored at −20° C. After samples from all time points were collected and frozen, the samples were thawed and centrifuged at 15 000 rpm for 10 min; each supernatant was collected, placed into a borosilicate tube, and dried in a Speedvac rotary evaporator. Each dried sample was redissolved in 200 μL of a 1:1 MeOH/H₂O solution, centrifuged to remove particulates, and then transferred to an HPLC vial. 50 μL of the sample was used for analysis on an Agilent 1260 Infinity series HPLC equipped with a Gemini 5 μM C-18 110 Å column (250×4.6 mm²) and a diode array detector. The sample was separated using a mobile phase consisting of 1% (v/v) triethylamine in H₂O and acetonitrile at the following ratios and time intervals: 80:20 for 2 min, a gradient from 80:20 to 2:98 over 15 min, continued 2:98 for 2 min, and then a gradient from 2:98 to 80:20 over 8 min continued 80:20 for 2 min. The reaction substrate and product were assessed by monitoring absorbance at 275 nm. 3a, 16e, and Atomoxetine were eluted at ~14, 12, and 15 min. A dilution series of known concentrations of authentic 3a, 16e, and Atomoxetine in 1:1 MeOH/H₂O was run to generate a standard curve and facilitate the conversion of product AUCs to absolute mass.

Quantification of 16e and 3a Levels in the Serum and Eves of Mice after Intraperitoneal Injection

8-week-old C57BL/6J mice were treated with 380 nmol of 16e in dimethylsulfoxide (50 μL) by intraperitoneal injection and sacrificed at 1, 4, or 24 h later. Vehicle alone (dimethylsulfoxide) and 380 nmol of 3a were injected as control, and the animals were sacrificed at 1 h. Blood and eyeball samples were collected. After clotting at room temperature for 30 min, the blood samples were centrifuged for 10 min at 17 000 g in a temperature-controlled benchtop centrifuge (Eppendorf AG). Each serum sample (100 μL) was carefully removed to avoid disturbing loose clots, precipitated with 100 μL of precooled methanol, and centrifuged at 1500 g for 15 min at 4° C. The supernatant was carefully transferred to a SpinX centrifuge tube filter with a 0.45 μm cellulose acetate membrane (Costar, Salt Lake City, UT) and centrifuged at 7000 g for 2 min. The filtered samples were dried under vacuum, reconstituted in 100 μL of 50% methanol/water, and centrifuged at 23 000 g for 15 min at 4° C. The resulting supernatants were ready for LC/MS analyses. The two eyeballs from each mouse were homogenized in precooled methanol (2×800 μL). The resulting mixture was centrifuged at 23 000 g for 15 min at 4° C. The supernatant was dried under vacuum, reconstituted in 100 μL of 50% methanol/water, and centrifuged at 23 000 g for 15 min at 4° C. Five microliters of the supernatant extracted from the serum or eye samples was injected into a Vanquish HPLC system coupled with a Q-Exactive mass spectrometer (Thermo Fisher Scientific, Waltham, MA) with an ESI unit. The separation was performed on a Proshell EC-18 column (2.7 μm, 3.0×150 mm², Agilent, Santa Clara, CA) using a mobile phase consisting of 0.1% aqueous formic acid (A) and acetonitrile (B) at a flow rate of 600 μL·min⁻¹, and the mobile-phase gradients and time course were as follows: 0-2 min, 95% A/5% B: 2-10 min, 95-15% A/5-85% B. The signals of emixustat were detected in the parallel reaction monitoring mode with 25% collision energy. The peaks represent the fragment at m/z 217.2 were quantified. The signals of 16e and 3a were detected in the selected ion monitoring mode. The peaks at 7.61 min with m/z 264.2 were quantified. The standards were prepared by using the matrix obtained from the plasma and the eye of untreated animals.

Data are presented as mean t SD, with the number of biological or technical replicates indicated in the figure legends. Curve fitting was performed by nonlinear least-squares methods as implemented in GraphPad Prism.

### RPE65 Crystallization and Structure Determination

Crystals of RPE65 in the complex with 16e were obtained using previously described procedures. Briefly, isolated bovine RPE membranes were incubated with 2.5 mM 16e |delivered in dimethylformamide (DMF)| for 15 min prior to solubilization with 24 mM hexaethylene glycol mono-octyl ether (C8E6). After anion-exchange chromatography, purified RPE65 was concentrated to 10-15 mg/mL and 16e was added again to a concentration of 2.5 mM prior to crystallization. Crystals were grown by the hanging-drop vapor diffusion method by mixing 2 μL of a 10 mg/mL RPE65 sample with 2 μL of crystallization solution consisting of 30% v/v PEG 400, 100 mM CAPS, pH 10.5, 500 mM (NH₄)₂SO₄, and 10% v/v glycerol. After incubation for 1-2 weeks at 8° C., crystals of ~100×100×300 μm³ in size were observed. Mature crystals were harvested directly into liquid nitrogen for X-ray data collection. X-ray diffraction data were collected at the APS NE-CAT 24-ID-E beamlines. Data were processed using XDS, and the initial model was obtained by direct refinement using published RPE65 coordinates in which ligands had been removed (PDB accession code: 4RYX). The structure was refined by alternating reciprocal space refinement in REFMAC and manual building and adjustments in Coot. Ligand coordinates and geometry dictionary files were generated using the Grade server. The models were validated using MolProbity and the wwPDB validation server.

### Results

### Synthesis

We organized our approach into four families of molecules. First, we aimed to investigate the geometric requirements of the γ-amino-α-alcohol recognition element with respect to the ether moiety. We were able to obtain all substituted (ortho and para) emixustat (here named 3a for simplicity) via a previously reported synthetic pathway (Scheme 1). Briefly, the commercially available phenols were reacted with potassium carbonate, (bromomethyl)-cyclohexane, and DMF to give benzaldehydes 1a-c. Subsequent aldol reaction between LDA and acetonitrile gave nitriles 2a-c, which were finally reduced via LAH reduction to give amines 3a-c.

### Scheme 1—Synthesis of Emixustat γ-Amino-α-Aryl Propanols

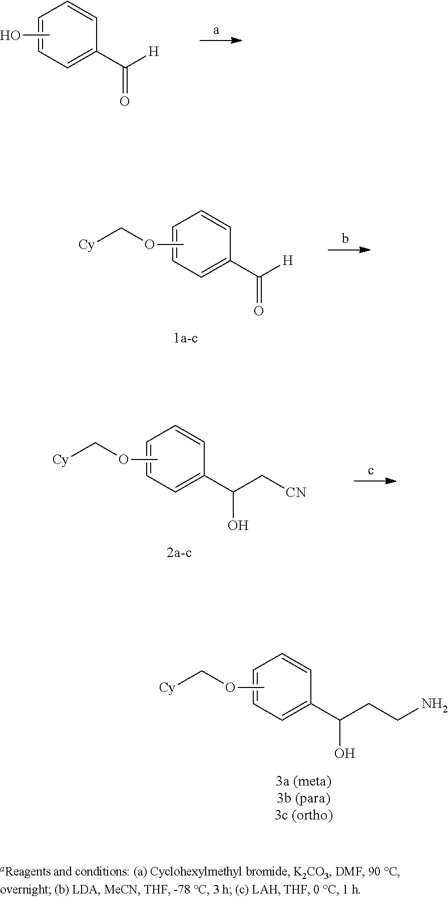

Next, we shifted our focus to probe how perturbations to the γ-aminopropanol moiety would affect RPE65 recognition. The second subset of molecules would relocate the hydroxyl to a different position on the propylene chain while at the same time conserving the amine moiety on the chain. A novel approach (Scheme 2) was employed to synthesize these three additional amino alcohols. Anhydrous HCl gas (generated in situ) was bubbled through a suspension of commercially available tyrosine derivatives in methanol. Subsequent Boc protection afforded the N-Boc-protected methyl esters 4a-c. Phenols 4a-c were deprotonated to undergo S_{N}2 displacement of (bromomethyl)cyclohexane to give esters 5a-c. Finally, consecutive ester reduction with LAH and Boc deprotection using trifluoroacetic acid gave target amino-propan-1-ols 6a-c.

### Scheme 2—Synthesis of γ- and β-Amino-γ-aryl Propanols^{α}

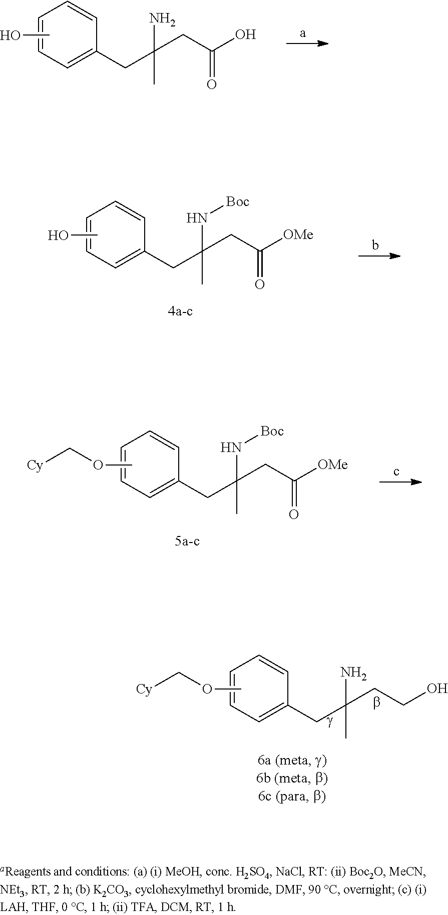

A third subfamily of molecules were synthesized that all possess a truncated aliphatic chain compared to emixustat. Amine 11a was synthesized following a previously reported method. Three amines were synthesized using a general method illustrated in Scheme 3. In short, benzaldehyde 1a or acetophenone 7 were reduced with sodium borohydride to give the corresponding benzylic alcohols 8a-b. These benzylic alcohols were converted to the analogous benzylic nitriles in two steps: first by reaction of alcohols with phosphorus tribromide, then substitution with potassium cyanide to give benzylic nitriles 9a-b. Final nitrile reduction using borane yielded the desired short-chain derivatives 11b-c. Finally, the fluorinated version of 11b was synthesized. We employed a Henry reaction between benzaldehyde 1e, nitromethane, and ammonium acetate to give the unsaturated nitro compound 10. Simultaneous olefin and nitro reduction was achieved by the addition of LAH under heat to give truncated amine 11d.

### Scheme 3—Synthesis of (3-Cyclohexylmethoxy)phenylethanamines^{α}

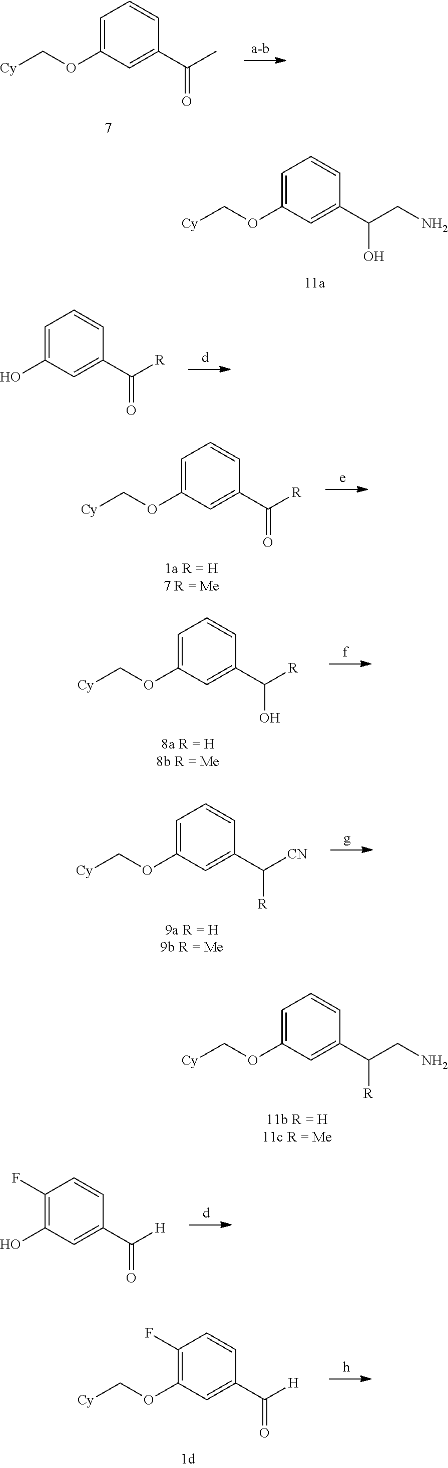

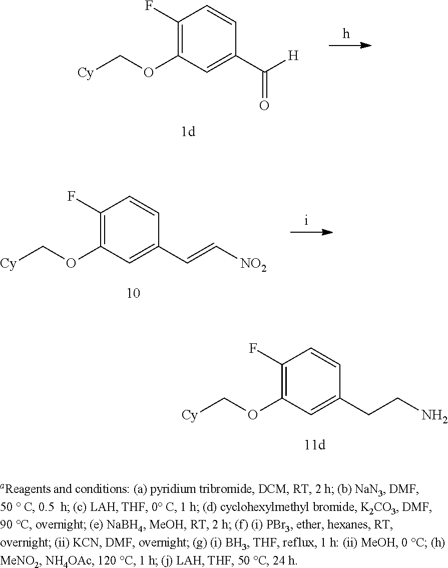

Lastly, we aimed to synthesize methylated derivatives of emixustat to maximize the structural correspondence between 3a and the retinoid backbone. First, amine 16a was synthesized according to literature precedent. In short, Sonogashira coupling of 2-(prop-2-yn-1)isoindoline-1,3-dione with aryl iodide 12 gave alkyne 13. Consecutive hydrogenation of the alkyne and then phthalimide deprotection with hydrazine gave propylamine 16a. As displayed in Scheme 4, novel synthetic pathways were taken to synthesize 16b-16e. We started with a Horner-Wadsworth-Emmons condensation between aceto-phenone 7, benzaldehydes 1a, or 1e and, with the appropriate phosphonates, the reaction afforded unsaturated nitriles 14-15a/b. Subsequent addition of LAH simultaneously reduced both the alkene and nitrile to give target amines 16b and 16c, respectively, and hydrogenation of nitrile 15b gave the desired fluorinated molecule 16d. Finally, methylamine 16e was readily made in two steps starting with the selective mesylation of diol MB-002 followed by displacement with methylamine in a sealed tube.

### Scheme 4—Synthesis of (3-Cyclohexylmethoxy)phenylpropanamines^{α}

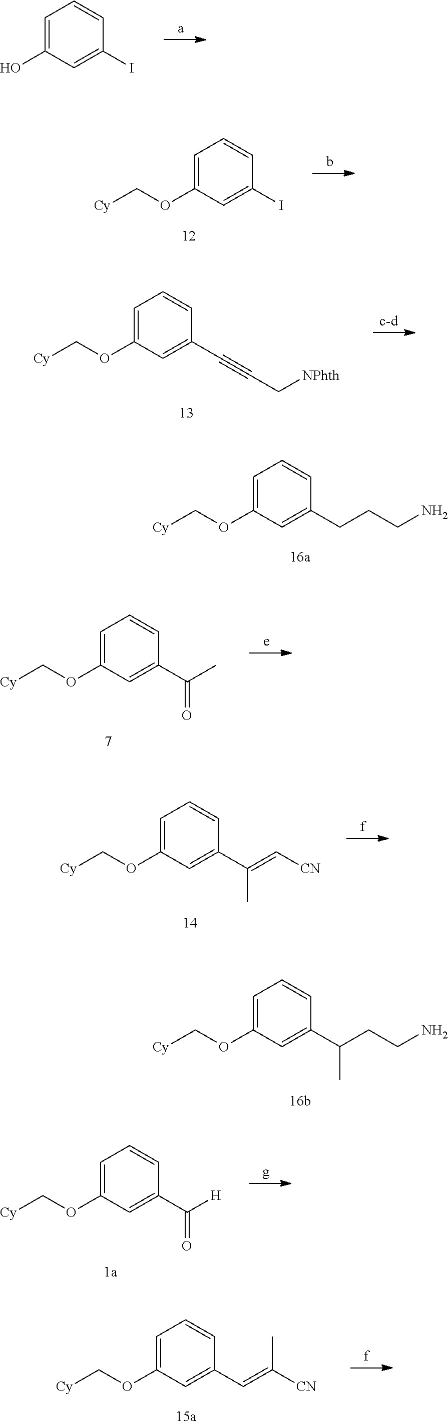

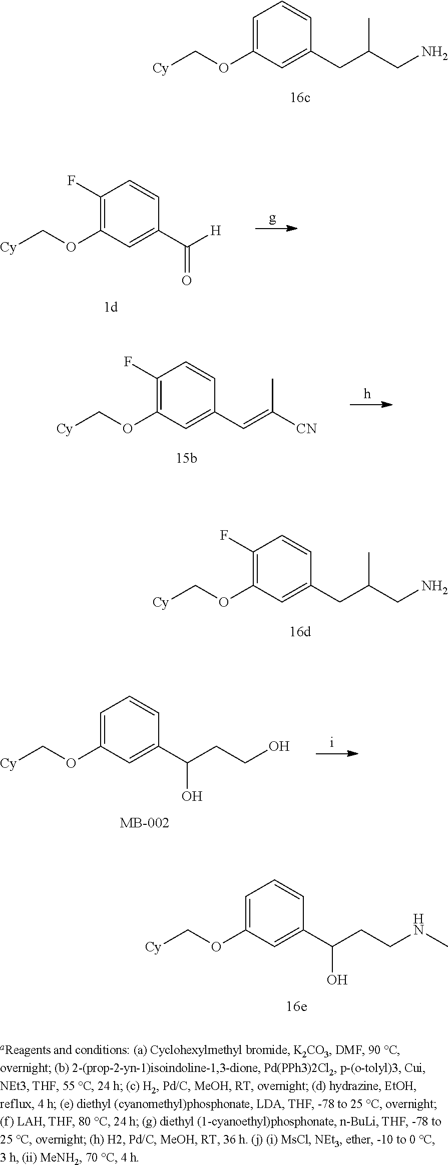

### RPE65 Inhibitory Properties of Tested Compounds

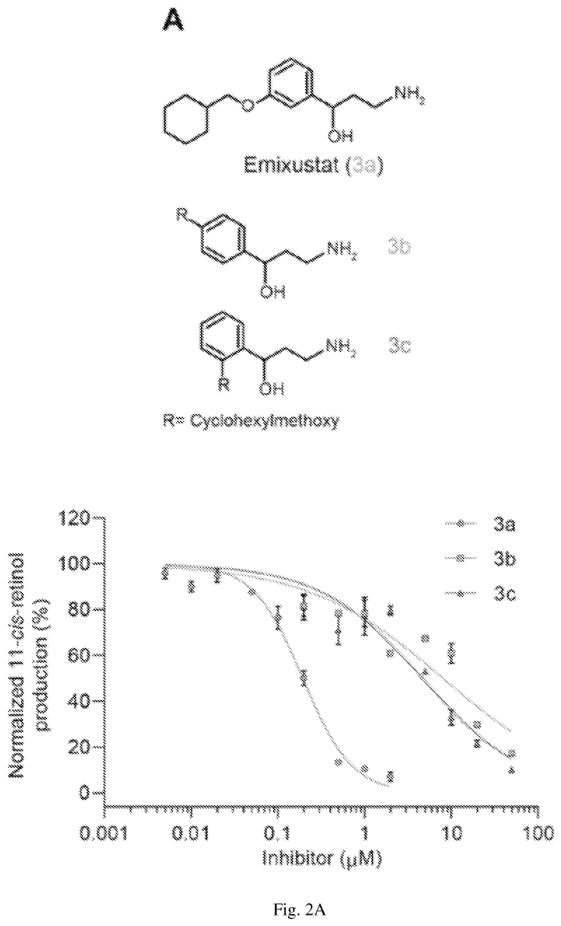

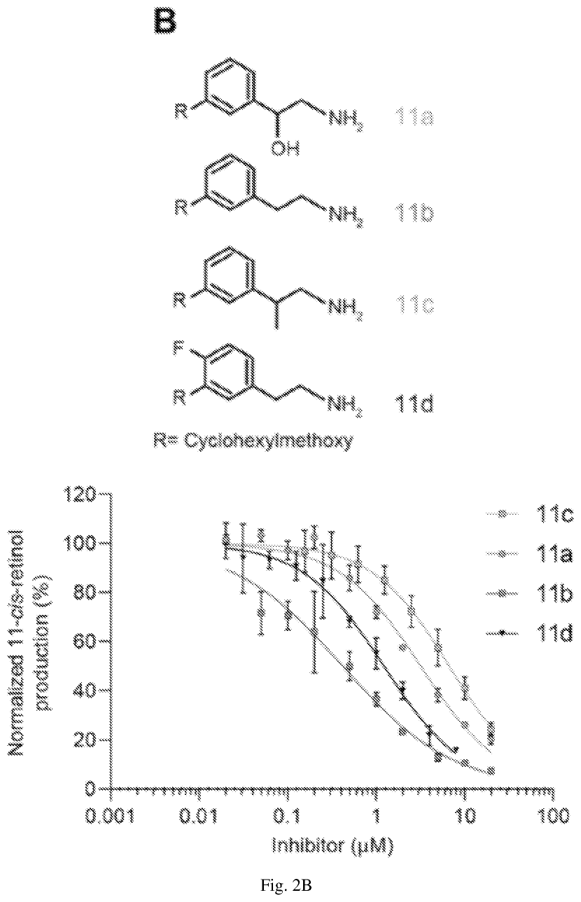

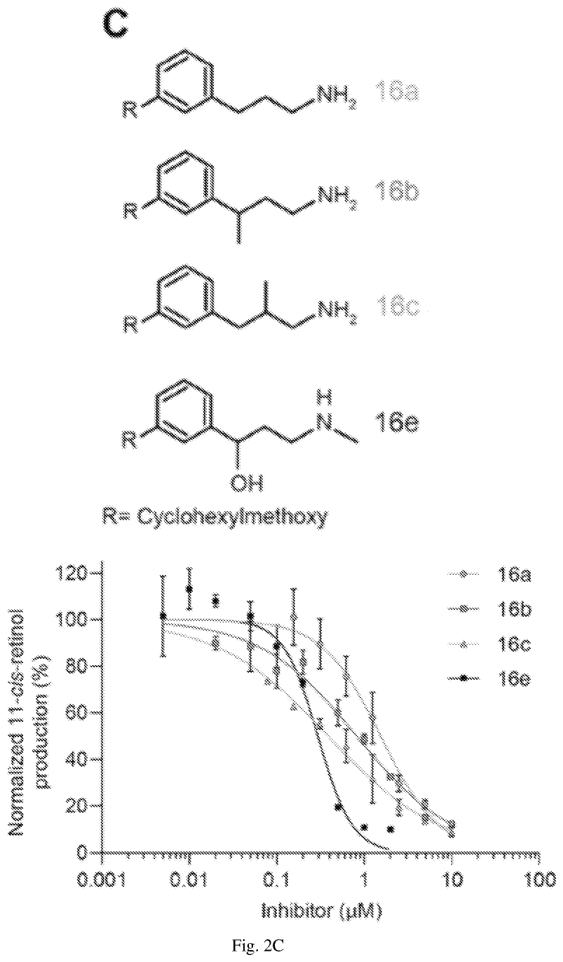

A total of 15 derivatives were tested for their inhibitory effects on RPE65 in vitro by monitoring their impact on 11-cis-retinol production by bovine RPE microsomes using established assay conditions (Table 1). First, because almost all studied emixustat derivatives to date have the γ-amino-α-aryl alcohol moiety meta relative to the six-membered ring, we assessed the effect of phenyl ring substitution pattern on RPE65 inhibition (FIG. 2A). Emixustat (3a) had a half-maximal inhibitory concentration (IC₅₀) of 200±20 nM, consistent with published data, whereas 3b (para) and 3c (ortho) analogues exhibited substantially higher IC₅₀ values (8.08 t 3.44 and 4.67 t 1.45 μM, respectively) suggesting that the bent geometry imparted through the meta configuration of emixustat is ideally complementary to the RPE65 active site, supporting our overall hypothesis that the most effective RPE65 inhibitors are isosteres of the 11-cis-retinyl cation transition state. Next, we tested the effect of swapping the position of the primary amine with the alcohol in the γ-amino-α-aryl alcohol moiety. Compounds 6a-c showed no inhibition of RPE65 within the concentration range of 0.1-50 μM. The lack of inhibitory activity observed for 6a was particularly striking given the known tolerance of alcohol substitution at the γ position. This outcome may be due to relatively poor stabilization of the amine functionality within the RPE65 active site, which is predicted to be protonated in our assay conditions, in the γ position compared to the α position. Interestingly, the lack of dose-response relationship observed for amine 6c also suggests that compounds in this family have no added potency enhancement by being in the para configuration.

TABLE 1
Structures and IC₅₀ Values of Tested Emixustat Derivatives^{a}

| | IC₅₀ ± |
|---|---|
| | SD |
| Structure | (μM) |
| 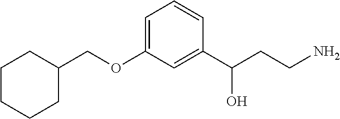 [SMILES: NCCC(O)c1cccc(OCC2CCCCC2)c1] | 6.20 ± 0.92 |
| 3a | |
| 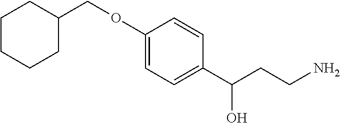 [SMILES: NCCC(O)c1ccc(OCC2CCCCC2)cc1] | 8.98 ± 3.44 |
| 3b | |
| 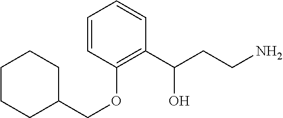 [SMILES: NCCC(O)c1ccccc1OCC1CCCCC1] | 4.67 ± 1.23 |
| 3c | |
| 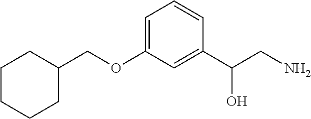 [SMILES: NCC(O)c1cccc(OCC2CCCCC2)c1] | 3.19 ± 0.81 |
| 11a | |
| 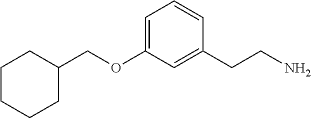 [SMILES: NCCc1cccc(OCC2CCCCC2)c1] | 0.42 ± 0.11 |
| 11b | |
| 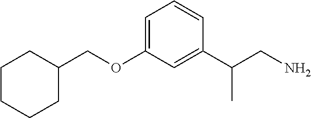 [SMILES: CC(CN)c1cccc(OCC2CCCCC2)c1] | 6.73 ± 0.26 |
| 11c | |
| 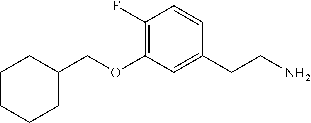 [SMILES: NCCc1ccc(F)c(OCC2CCCCC2)c1] | 1.23 ± 0.13 |
| 11d | |
| 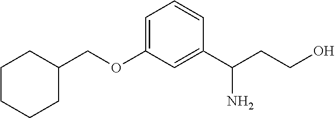 [SMILES: NC(CCO)c1cccc(OCC2CCCCC2)c1] | No Inhibition |
| 6a | |
| 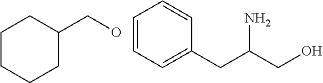 | No Inhibition |
| 6b-meta | |
| 6c-para | |
| 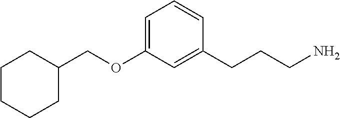 [SMILES: NCCCc1cccc(OCC2CCCCC2)c1] | 1.55 ± 0.3 |
| 16a | |
| 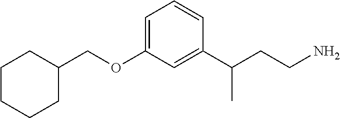 [SMILES: CC(CCN)c1cccc(OCC2CCCCC2)c1] | 0.82 ± 0.5 |
| 16b | |
| 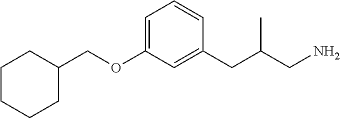 [SMILES: CC(CN)Cc1cccc(OCC2CCCCC2)c1] | 0.59 ± 0.07 |
| 16c | |
| 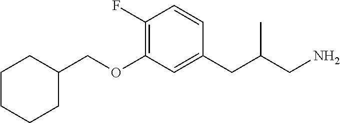 [SMILES: CC(CN)Cc1ccc(F)c(OCC2CCCCC2)c1] | No Inhibition |
| 16d | |
| 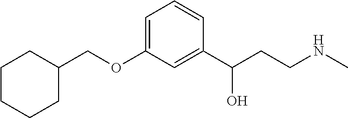 [SMILES: CNCCC(O)c1cccc(OCC2CCCCC2)c1] | 0.29 ± 0.05 |
| 16e | |

^{a}Results are expressed as mean ± SD, n = 3.

Next, we investigated the series of emixustat derivatives with truncated γ-amino-α-aryl alcohol (FIG. 2B). We hypothesized that this modification may impact inhibitory activity by increasing or decreasing the distance between the primary amine of the inhibitor and Glu148 and palmitoyl-associated carboxylate moieties (FIG. 1C). Compounds 11a-d were tested in the concentration range of 0.02-20 μM. Amine 11a was substantially less potent than 3a (IC₅₀=3.19±0.81 μM) suggesting that the proper positioning of the cationic amine is crucial for optimal active site interaction. With this observation, we sought to probe the 2-amino-1-hydroxyethane chain of amine 11a to determine if further modifications would affect inhibitory potency. Removal of the hydroxyl moiety gave 11b (IC₅₀=420±110 nM), a compound with 7.6-times more potent activity toward RPE65 relative to amine 11a. Substitution of the alcohol with a methyl group yielded 11c, with weaker inhibition (IC₅₀=6.73±0.26 μM) likely arising from nonfavorable interactions with the Thr147 hydroxyl moiety. This result was somewhat unexpected as one of the hypotheses we sought to test throughout the series described here was whether a mimic of the retinoid C20 methyl group would impart enhanced activity. Instead, it resulted in low RPE65 inhibition, suggesting that this molecule cannot occupy RPE65's active site like the retinyl cation does. Fluorinated compound 11d (IC₅₀=1.23±0.13 μM) notably showed a 3-fold decrease in RPE65 inhibition relative to 11b. This result was incongruent with our previous findings that aryl fluorination at this position should impart higher affinity toward RPE65. These results together suggest that 11b and 11d do not occupy the same binding position as emixustat relative to the iron center of RPE65. Also, these data indicate that, at least in vitro, the hydroxyl group of the γ-amino-α-aryl alcohol moiety is not obligatory for RPE65 inhibition.

Next, we applied the same logical considerations of 11a-d to the γ-amino-α-aryl alcohol recognition element. Specifically, we tested the regiospecific incorporation of a methyl group to generate compounds 16a-d. These compounds were tested in the concentration range between 0.02-10 μM. Plotted in FIG. 2C, amine 16a (IC₅₀=1.55 t 0.30 μM) and 16b (IC₅₀=820 t 150 nM), showed decreased activity toward RPE65 relative to 3a with an 8-fold and 4-fold decrease in inhibition of RPE65, respectively. These results indicate that the addition of only a methylene unit compared to 11b decreases RPE65 inhibition, suggesting the critical nature of the proper placement of the amino group. Again, in aiming to mimic the retinoid C20 methyl group (as with 11b and 11c) we found that methylation at the α and β-carbons (16b and 16c, respectively) roughly doubled and quadrupled potency relative to 16a. The enhanced potency of 16c relative to 16b may arise from the more ideal structural correspondence between the β-methyl and retinoid C20 methyl groups. It is important to note that 16c achieves this level of potency without the α-hydroxyl moiety found in emixustat. Again, using the rationale as above, we generated a fluorinated derivative of 16c (16d) but it was inactive.

Collectively, these results demonstrate that a γ-amino-β-methyl moiety is sufficient to attain significant RPE65 inhibition in vitro. However, they also highlight the critical spacing and composition of the γ-amino-α-aryl alcohol recognition element of 3a with RPE65 (FIG. 1C).

Lastly, we explored the effect of methylating the primary amine of 3a on RPE65 inhibition. We were initially surprised that amine 16e (IC₅₀=290±50 nM) retains most of the activity of emixustat, as our prediction was that the N-methyl functionality would significantly obstruct the crucial interaction of the ammonium as described in our previous structure of emixustat with RPE65. Nevertheless, this was a crucial result as it showed that amine methylation does not prevent potent inhibition of RPE65 in vitro.

### Inhibition of RPE65 In Vivo

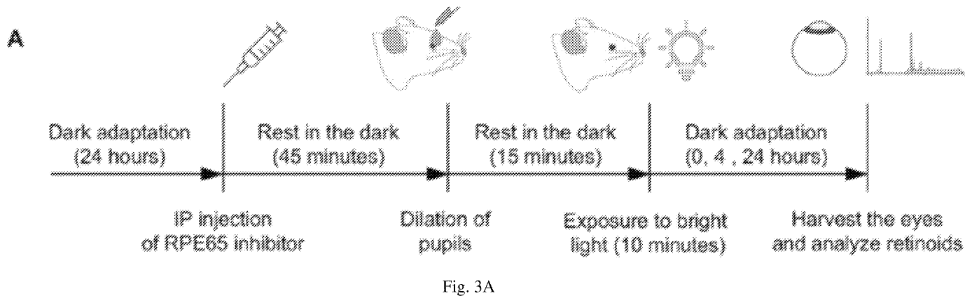

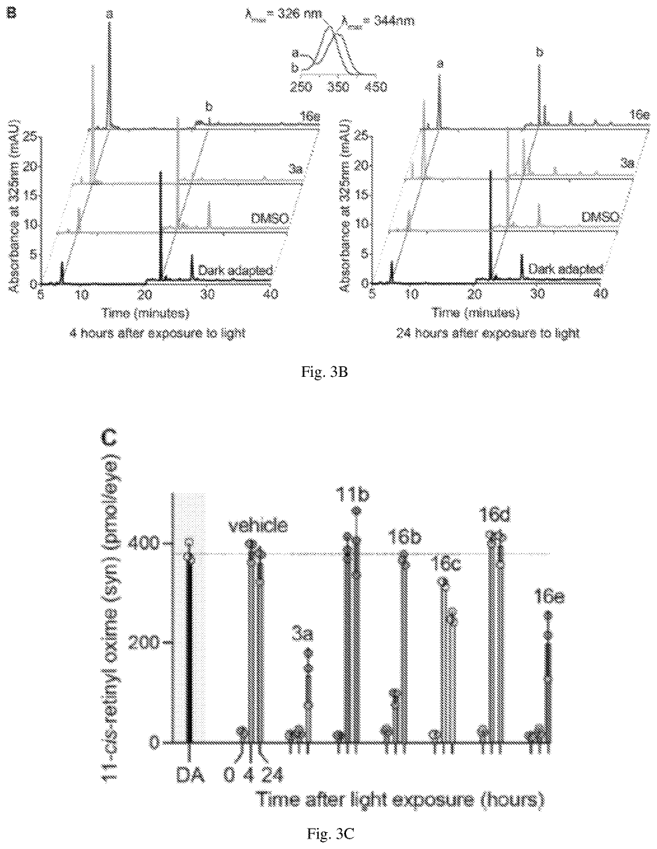

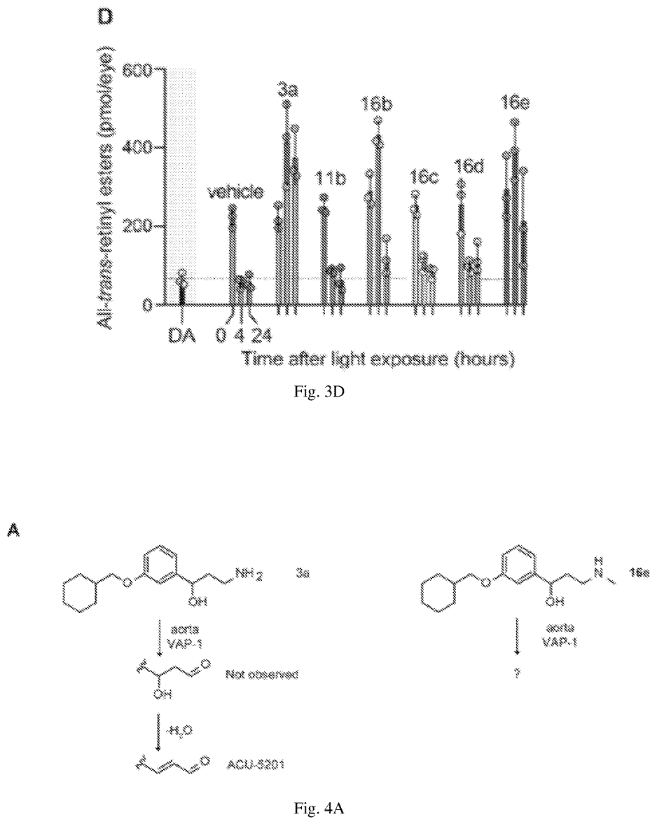

Next, we tested the effect of the most potent compounds (11b and 16b-e) on visual cycle function in BALB/cJ mice. FIG. 3A illustrates the experimental procedure used in this study. FIG. 3B (left) shows representative chromatograms obtained from animals 4 h after treatment with 3a (blue trace), 16e (purple trace), and DMSO (green trace). Compared to the dark-adapted (DA) (black trace) and the vehicle-treated chromatograms, both 3a and 16e groups showed large accumulation of RE accompanied by significantly reducing recovery of 11cRAL. Peaks corresponding to 11cRAL and RE were identified by retention time and UV-Vis absorption spectrum (FIG. 3B, inset). FIG. 3B (right) shows representative chromatograms obtained from the same groups but 24 h after treatment with 3a (blue trace), 16e (purple trace), and DMSO (green trace). One day after injection, 11cRAL and RE levels were not yet recovered in the groups treated with 3a and 16e.

The quantification of 11cRAL and RE for each group is summarized in Table 2. Baseline levels of 11cRAL (FIG. 3C, black bar) and RE (FIG. 3D, black bar) were established in dark-adapted (DA) animals were 384.1±15.2 and 67.4±12.4 pmol/eye, respectively. In the groups treated with vehicle (FIG. 3C,D, green bars) and 16d (FIG. 3C,D, gray bars), 4 h of dark adaptation were sufficient to restore baseline levels of both 11cRAL and RE, 389.5±17.2 and 58.2±11.0 pmol/eye, in the vehicle group, respectively; 398.0±26.6 and 122.7±30.6 pmol/eye in the 16d group, respectively.

TABLE 2
^{a}Concentrations of 11cRAL and RE in Mice Eyes Quantified by HPLC

| | Time | Mean ± SD | Mean ± SD |
|---|---|---|---|
| Group | (hours) | 11cRAL (pmol/eye) | RE (pmol/eye) |
| dark adapted | DA | 384.1 ± 15.2 | 67.4 ± 12.4 |
| vehicle | 0 | 22.7 ± 2.6 | 223.9 ± 20.4 |
| | 4 | 389.5 ± 17.2 | 58.2 ± 11.0 |
| | 24 | 363.5 ± 26.5 | 60.0 ± 15.0 |
| 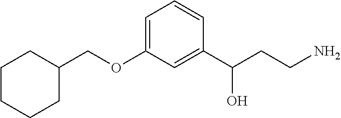 [SMILES: NCCC(O)c1cccc(OCC2CCCCC2)c1] | 0 4 24 | 16.2 ± 16.2 22.3 ± 4.0 137.4 ± 44.5 | 225.1 ± 29.5 416.8 ± 86.4 377.3 ± 54.5 |
| 3a | | | |
| 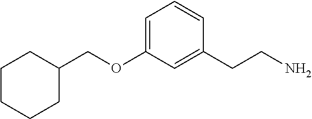 [SMILES: NCCc1cccc(OCC2CCCCC2)c1] | 0 4 24 | 16.2 ± 16.2 22.3 ± 4.0 137.4 ± 44.5 | 225.1 ± 29.5 416.8 ± 86.4 377.3 ± 54.4 |
| 11b | | | |
| 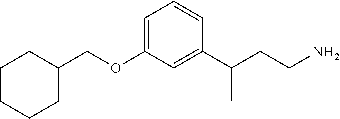 [SMILES: CC(CCN)c1cccc(OCC2CCCCC2)c1] | 0 4 24 | 25.6 ± 2.7 94.4 ± 11.4 370.5 ± 9.1 | 291.5 ± 33.1 435.5 ± 27.5 123.9 ± 36.4 |
| 16b | | | |
| 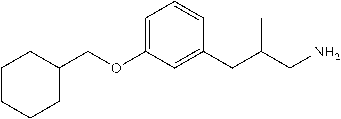 [SMILES: CC(CN)Cc1cccc(OCC2CCCCC2)c1] | 0 4 24 | 17.4 ± 0.8 323.0 ± 5.1 253.5 ± 9.0 | 253.5 ± 22.5 111.9 ± 18.5 85.6 ± 12.7 |
| 16c | | | |
| 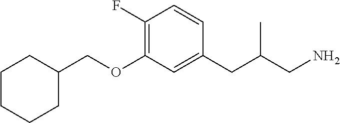 [SMILES: CC(CN)Cc1ccc(F)c(OCC2CCCCC2)c1] | 0 4 24 | 23.5 ± 2.3 413.2 ± 8.1 398.0 ± 26.6 | 259.9 ± 54.6 101.1 ± 12.9 122.7 ± 30.6 |
| 16d | | | |
| 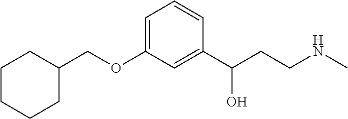 [SMILES: CNCCC(O)c1cccc(OCC2CCCCC2)c1] | 0 4 24 | 13.8 ± 1.5 22.5 ± 4.5 201.8 ± 53.2 | 296.6 ± 65.2 393.5 ± 60.0 213.4 ± 99.2 |
| 16e | | | |

^{a}Results are expressed as mean ± SD, n = 3 mice.

By contrast, 24 h post-treatment with 3a (FIG. 3C, D, blue bars), the level of 11cRAL was significantly lower compared to the DA group and vehicle groups, 137.4144.5 pmol/eye, which corresponds to 36% of the DA level. Consistent with suppressed 11cRAL synthesis, RE levels were significantly increased compared to the DA group at 377.3±54.5 pmol/eye. This result corroborated the inhibition of RPE65 activity we observed in bovine RPE microsomes (FIG. 2A) and demonstrated strong RPE65 inhibition in vivo as reported previously. Treatment with 16e (FIG. 3C, D) also showed potent RPE65 inhibition, consistent with in vitro measurements. 24 h after administration of 16e, 11cRAL and RE levels were 201.8±53.2 and 215.4±99.2 pmol/eye, respectively. Notably, the concentration of 11cRAL per eye was 53% of the DA. This result suggested that mono-methylation of the emixustat did not alter the ocular targeting. Treatment with compound 11b (FIGS. 3C, D) did not display hallmarks of inhibited RPE65 activity in vivo. Both 11cRAL and RE levels were completely recovered 4 h after exposure to light, 393.2±18.2 and 89.5±5.2 pmol/eye for 11b, respectively. This result differed from our measurements of RPE65 inhibition in vitro (FIG. 2B) indicating that 11b did not accumulate sufficiently in the RPE in vivo. Thus, truncation of γ-amino-α-aryl alcohol moiety together with the removal of the alcohol is not a viable strategy for the inhibition of RPE65 in vivo.

Compounds 16c (FIGS. 3C, D, yellow bars) and 16b (FIGS. 3C, D) showed minor RPE65 inhibition 4 h after treatment, but animals were fully dark-adapted after 24 h. 11cRAL and RE levels were 370.5±9.1 and 123.9±36.4 pmol/eye for 16c, respectively, and 253.5±9.0 and 85.6±12.7 pmol/eye for 16b, respectively. We note that the visual cycle inhibition observed for 16b may be due to nonspecific toxicity as all animals showed impaired body movement even 24 h after treatment.

To summarize, all tested compounds that failed to inhibit RPE65 in vivo were primary amines without an alcohol. By contrast, the inhibition profile of the secondary amine 16e resembled that of emixustat. Taken together, these results demonstrated that both the amine and alcohol of the γ-amino-α-aryl alcohol moiety are required to attain sustained RPE65 inhibition in vivo.

### Pharmacokinetics of Compound 16e

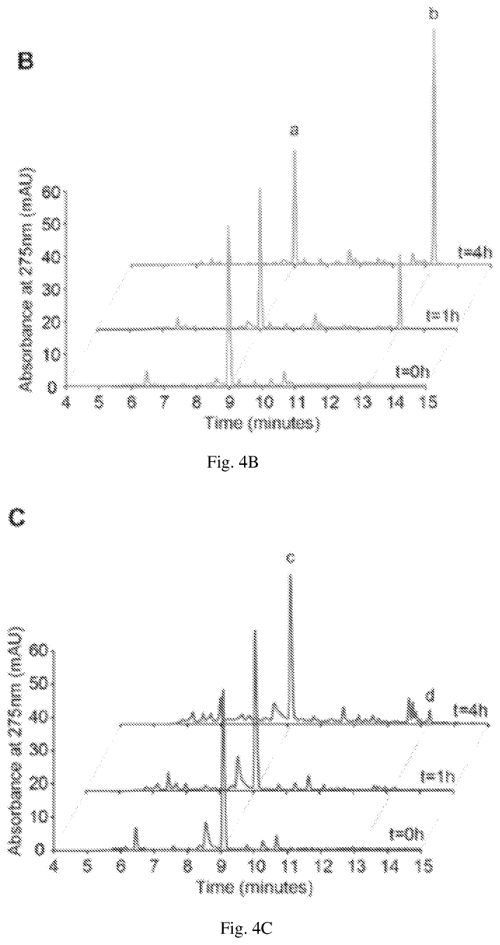

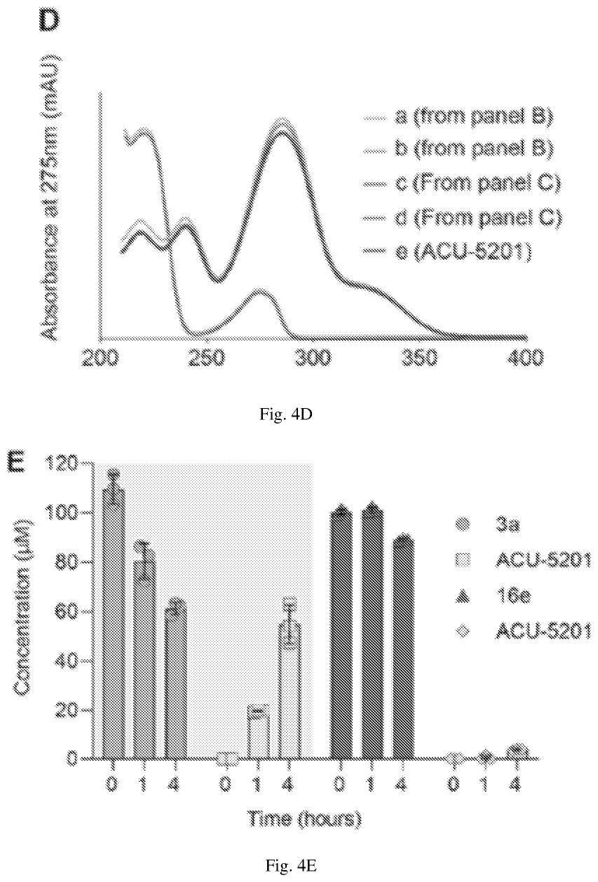

Next, we investigated the stability of 16e to oxidative metabolism in vitro with an HPLC-based activity assay using mouse aorta homogenates as the source of VAP-1 enzyme. Previous investigations highlighted that the elimination of emixustat in vivo is prominently driven by VAP-1 present in the vasculature. In vitro experiments identified ACU-5201 aldehyde as the major emixustat product of VAP-1 catalytic activity (FIG. 4A). Because VAP-1 exerts oxidative deamination selectively toward primary amines, the secondary amine of compound 16e would be predicted to be insensitive to VAP-1 enzymatic activity. We tested this hypothesis by comparing the rate of oxidative deamination of 3a vs that of compound 16e. The formation of the dehydrated aldehyde product (ACU-5201) was monitored by reverse-phase HPLC (FIGS. 4B, C) by comparison of its retention time and UV-vis spectra to that of the authentic synthetic standard ACU-5201 (FIG. 4D) (synthesis of ACU-5201 reported previously by us).

Quantitative analysis is illustrated in FIG. 4E, and numerical results are summarized in Table 3.

TABLE 3
Concentration of Tested Compounds and Their Deaminated
Product in Mouse Aorta Homogenates Quantified by HPLC^{a}

| | | mean ± SD | | mean ± SD |
|---|---|---|---|---|
| time | mean ± SD | ACU- [symbol] 201 | mean ± SD | ACU- [symbol] 201 |
| (h) | 3a (μM) | (μM) | 16e (μM) | (μM) |
| 0 | 109.47 ± 4.95 | 0.00 ± 0.00 | 100.32 ± 0.95 | 0.00 ± 0.00 |
| 1 | 80. [symbol] 3 ± [symbol] .85 | 19.71 ± 0.29 | 101.20 ± 0.89 | 0.48 ± 0.68 |
| 4 | 61.32 ± 2.06 | [symbol] 4.81 ± 6.38 | 89.07 ± 0.49 | 3.76 ± 0.29 |

^{a}Results are expressed as mean ± SD, n = 3 technical replicates. [symbol]
indicates data missing or illegible when filed

As expected, 3a was rapidly metabolized by VAP-1. After 1 h of incubation. 18% of emixustat was metabolized to ACU-5201, which increased to 50% after 4 h, similar to our previous findings. By contrast, mono-methylation of the γ-amino-α-aryl alcohol moiety (compound 16e) reduced the formation of ACU-5201 by about 99.5% after 1 h and 96.25% after 4 h.

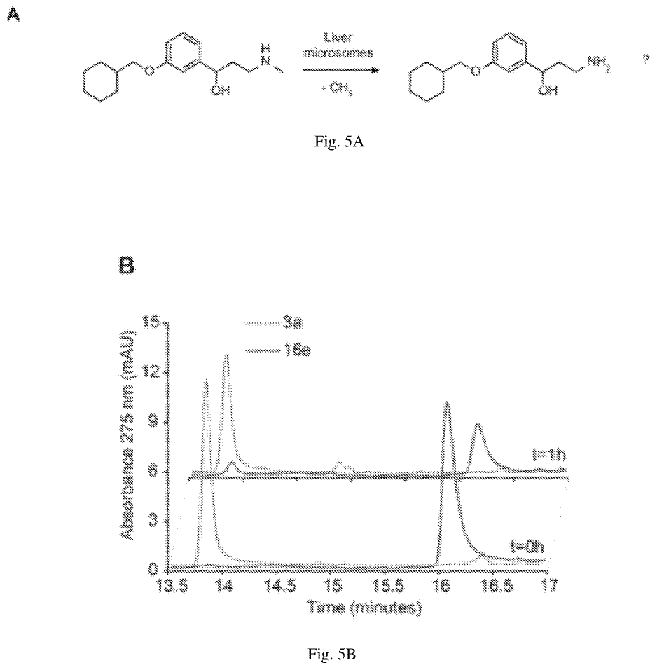

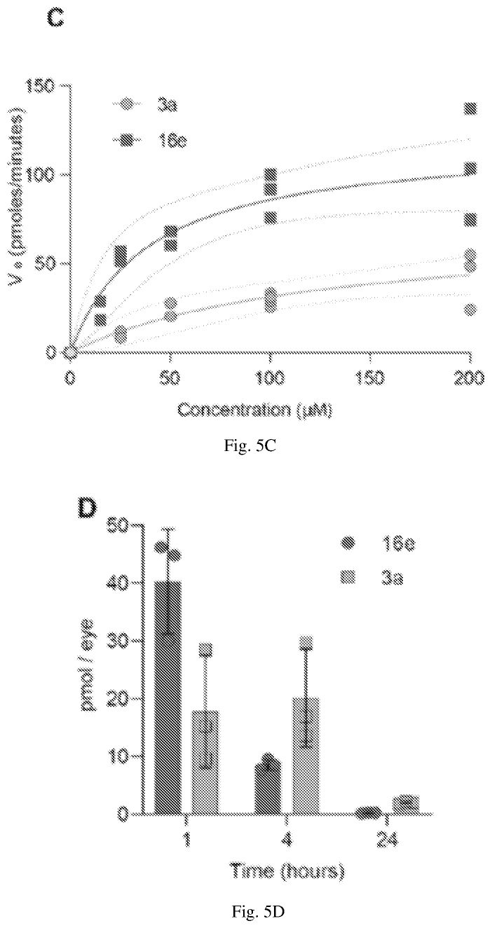

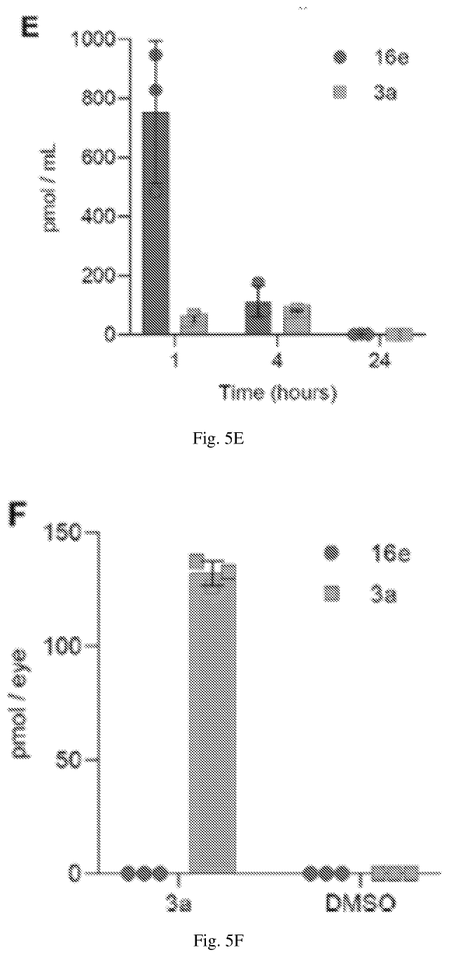

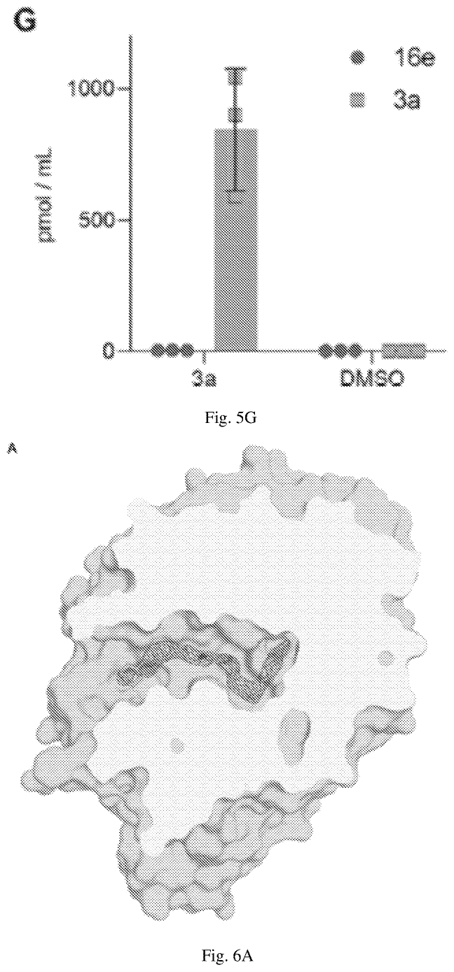

We next studied whether 16e is a prodrug of 3a, as demethylation of the secondary amine by enzymes of the CYP-450 family would generate 3a (FIG. 5A) which is a strong RPE65 inhibitor per se (FIG. 3B,C and Table 2). To test this possibility, we investigated the metabolism of compound 16e in vitro with an HPLC-based activity assay using mouse liver homogenates as the source of CYP-450 enzymes. Compounds 16e or 3a were added at different concentrations (0.15-200 μM) to a fixed amount of mouse liver microsomes and the kinetics of the reactions were followed at time points ranging from 0 to 1 h (FIGS. 5B, C). This assay was validated by a negative control reaction in which the NAPDH cofactor required for CYP-450 activity was omitted. The numerical results from this experiment are summarized in Table 4.

TABLE 4
Estimated V₀ of Tested Compounds in Mouse
Liver Microsomes Quantified by HPLC^{a}

| concentration | mean ± SD 3a | mean ± SD 16e |
|---|---|---|
| (μM) | (pmol/min) | (pmol/min) |
| 0 | 0.00 ± 0.00 | 0.00 ± 0.00 |
| 15 | not tested | 23.3 [symbol] ± [symbol] .32 |
| 25 | 10.08 ± 1.71 | 54.17 ± 2.78 |
| 50 | 24.05 ± 3.69 | 64.21 ± 3.84 |
| 100 | 29.67 ± 2.97 | 89.82 ± 8.70 |
| 200 | 4 [symbol] .92 ± 11.77 | 97.4 [symbol] ± 2 [symbol] .77 |

^{a}Results are expressed as mean ± SD. [symbol]
indicates data missing or illegible when filed

One-hour exposure of 200 μM 16e to liver microsomes (purple trace) elicited a minor amount of demethylation as demonstrated by the presence of the peak corresponding to 3a compared to the chromatogram of the reaction run with 3a (blue trace). However, in vitro, this metabolic pathway accounted for a small amount of the clearance of 16e. We observed the rapid appearance of another metabolite of 16e, which eluted earlier compared to 16e, indicative of a higher hydrophilicity compared to that of the parent compound 16e. However, this metabolite was not readily amenable to precise chemical identification by mass spectrometry. Notably, this metabolite of 16e was not observed for emixustat, indicating that mono-methylation of the primary amine in the RPE65 recognition motif conferred susceptibility to a new elimination pathway. Additionally, we did observe the formation of ACU-5201 from emixustat in this mouse liver microsome assay, indicating that the liver may also play a role in the oxidative deamination of this molecule in addition to the general vasculature although ACU-5201 was observed only in trace amount.

Our microsomal metabolism data allowed us to determine the intrinsic clearances (Cᵢₙₜ) of 16e and 3a. We derived the initial velocity (V₀) values at substrate concentrations in the range of 0.15-200 μM and fit these data with the Michaelis-Menten equation to derive steady-state kinetic constants and intrinsic clearance (Vₘₐₓ/Kₘ) values (Table 5). As shown in FIG. 5C, 16e is characterized by a higher Vₘₐₓ compared to 3a, 70.85 and 119.5 pmol/min, respectively, hence indicating that in the liver 16e was metabolized at a 1.7 times faster rate than 3a. This finding was consistent with a larger Kₘ for 3a compared to 16e, 126.3 and 39.01 pmol/mL. Finally, the Cᵢₙₜ of 16e was 1.83 L/mg while that of 3a was 0.34 L/mg. Thus, the methylation of the primary amine in the RPE65 recognition motif of emixustat imparted a 5.5 times higher hepatic clearance of 16e as compared to 3a.

TABLE 5
Pharmacokinetic Parameters of Tested Compounds
from Testing with Liver Microsomes^{a}

| parameter | mean ± S [symbol] 3a | mean ± S [symbol] 16e |
|---|---|---|
| V [symbol] (pmol/min) | 70.85 ± 48.97 | 119. [symbol] ± 15.99 |
| [symbol] (pmol/mL) | 126. [symbol] ± 192.3 [symbol] | 39. [symbol] ± 1 [symbol] .8 [symbol] |
| C [symbol] (L/h/mg) | 0.34 ± 0.5 [symbol] | 1.8 [symbol] ± 0.88 |

^{a}Results are expressed as mean ± S [symbol] .
C [symbol] values are normalized to the grams of protein in the amount of mouse liver microsomes used in assay. [symbol]
indicates data missing or illegible when filed

This result raised the question about the extent to which this minor in vitro conversion of 16e to 3a was relevant in vivo. To answer this question, we followed the progress of 16e elimination in the mouse eye and serum after a single IP injection of 16e in DMSO (50 μL, 10 mg/kg). In this experiment, samples were collected at 1, 4, and 24 h post-administration. We also included two control groups, animals treated with a single IP injection of 3a in DMSO (50 μL, 10 mg/kg) or vehicle alone (50 μL, DMSO). The collection of the sample from the control groups was limited to 1 h after IP injection. Numerical results from these experiments are summarized in Table 6.

TABLE 6
Quantification of 16e and 3a after Intraperitoneal
Injection of 16e or 3a^{a}

| | eye | | serum | |
|---|---|---|---|---|
| | mean ± | mean ± | mean ± | mean ± |
| time | SD 16e | SD 3a | SD 16e | SD 3a |
| ( [symbol] ) | (pmol/eye) | (pmol/eye) | (pmol/mL serum) | (pmol/mL serum) |

Quantification of 16e and 3a After Single IP of 16e

| | | | | |
|---|---|---|---|---|
| 1 | [symbol] . [symbol] 78 ± | 17.0 [symbol] ± | [symbol] .42 [symbol] = | [symbol] .34 [symbol] ± |
| | [symbol] .270 | [symbol] 885 | 196.201 | [symbol] .247 |
| 4 | [symbol] . [symbol] 09 ± | 19.454 ± | 111.11 [symbol] ± | [symbol] .5 [symbol] ± |
| | 0. [symbol] | 6.889 | 4 [symbol] . [symbol] | [symbol] .448 |
| 24 | [symbol] 000 ± | [symbol] 9 ± | 0.000 ± | 0.000 ± |
| | 0. [symbol] 00 | 0.2 [symbol] 8 | 0.000 | 0.000 |

Quantification of 16e and 3a After Single IP of 3a

| | | | | |
|---|---|---|---|---|
| 1 | 0.000 ± | 13 [symbol] . [symbol] 92 ± | 0.00 ± | [symbol] 40.9 [symbol] 7 ± |
| | 0.000 | 4. [symbol] | 0.000 | 189.28 [symbol] |

^{a}Abbreviations: IP, intraperitoneal [symbol] [symbol]
indicates data missing or illegible when filed

One hour after injection, 16e was found in eye (FIG. 5D, purple bars) and in plasma (FIG. 5E, purple bars) extracts, thus confirming the distribution of this visual cycle modulator in the ocular tissue. 16e levels decreased significantly at 4 h and approached baseline levels in both eye and plasma at 24 h after injection, suggesting elimination of 16e from these compartments. In the same animals, 3a (blue bars) was quantifiable at all of the time points that we investigated. This result demonstrated 16e demethylation in vivo, but its RPE65 inhibition was not representative of 16e being a prodrug of 3a, most clearly illustrated by the 4 h time point where 16e was still present at significant concentrations in the eye. FIGS. 5D, E also illustrates the most problematic pharmacokinetic property of 3a/emixustat, namely that this molecule will persist in ocular tissues for long time periods, leading to the main side effect noted in clinical trials, nyctalopia. Although we found that the conversion to 3a was significant, as illustrated by the relative concentrations at the 4 h time point, what was converted to 3a persisted for only 24 h. In contrast, in a previous study, we showed that IP injection of an equivalent dose of 3a led to a persistence of significant 3a concentrations in the eye for up to 7 days post-injection. Hence, the almost absent levels of 3a in mouse eyes 24 h after 16e administration were indicative of shorter duration of action of this compound compared to 3a. Altogether, these results demonstrate that compound 16e, besides being a potent RPE65 inhibitor, was also resistant to VAP-1 oxidative deamination. However, VAP-1 resistance came at the cost of increased hepatic metabolism and minor conversion to 3a.

Crystal Structure of RPE65 in Complex with 16e

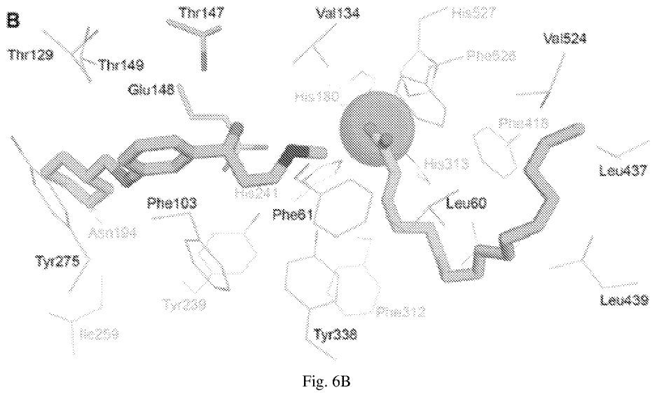

To gain an understanding of how 16e can be accommodated within the RPE65 active site cavity, we determined the crystal structure of RPE65 in complex with 16e in space group P6₅22 to a nominal resolution of 2.1 Å (FIG. 6A and Table 7). The data showed clear evidence for binding of 16e and a palmitate ligand in the proximal and distal regions of the RPE65 active site, respectively (FIG. 6B), similar to the modes of binding observed in other RPE65-ligand complexes. The terminal methyl group was observed to point toward the palmitate carboxylate carbon atom with a C—C interaction distance of ~3 Å. To confirm the methyl group positioning, we deleted that region of the model, performed 10 cycles of restrained refinement, and then inspected the resulting difference map. Consistent with our initial interpretation, we observed a strong difference map peak at the position of the deleted methyl group that remained despite shifts in the ligands that were made by the refinement program in an attempt to quench the difference map peak. Comparison of our final refined RPE65-16e complex with a previously reported RPE65-emixustat complex in the same space group (PDB accession code: 4RYX) revealed a similar mode of inhibitor binding indicating that the terminal methyl group is readily accommodated within the RPE65 active site.

The results discussed herein address three important aspects of targeted RPE65 inhibition. First, we have comprehensively defined in vitro and in vivo structure-activity relationships of the γ-amino-α-aryl alcohol recognition element of emixustat, a region of the molecule we have argued is critical for RPE65 active site recognition. Second, we have identified a key modification to the emixustat molecule that largely abrogates a major phase 1 metabolic pathway for emixustat elimination that is mediated by VAP-1 amine oxidase, though enhancing overall hepatic intrinsic clearance. Third, we have demonstrated with 16e that the key pharmacophore for RPE65 can be expanded to include modifications of the amine portion of the recognition element. Together, these data provide a capstone to our efforts to tune the PD/PK properties of emixustat to achieve a more therapeutically viable molecule. Indeed, emixustat-induced RPE65 inhibition is extremely long-lived due to ocular accumulation, a property that complicates the posology of emixustat. The faster metabolism of 16e in vitro and in vivo is possibly due in part to the increased hydrophobicity of this compound compared to emixustat. Therefore, our results establish a new strategy to control the duration of action of visual cycle modulators based on emixustat scaffold. Primary amine visual cycle modulators have been proposed to exert protective effects against light-induced retinal damage through two distinct mechanisms: (1) suppression of visual cycle kinetics via direct inhibition of RPE65 and (2) sequestration of reactive retinaldehyde through Schiff base formation. Because 16e is a secondary amine, Schiff base formation is expected to be less efficient compared to primary amine compounds. Therefore, we expect any protective effect exerted by 16e would occur through a direct visual cycle suppression mechanism. Likewise, we also expect that the secondary amine of 16e will reduce or exclude its susceptibility to LRAT-mediated amidation, which is proposed to be responsible for the long duration of action of retinylamine and emixustat. In the future, the complete elucidation of 16e's pharmacological effects on the physiology of vision will establish if this compound holds translational potential for the treatment of retinopathies.

In this example, we explored the structural requirements of what we have termed the RPE65 recognition element. Given the hypothesis that effective inhibition of RPE65 is most easily achieved via an amine-containing molecule to mimic the carbocation transition state of the retinoid isomerization reaction, better strategies were needed to address the metabolic liability of the primary amine of emixustat. The metabolism of primary amine drugs by VAP-1 is a lesser known phase I pathway, but has nevertheless been shown important for a number of primary amine-containing drugs besides emixustat including primaquine and tresperimus. Previously, we argued that deuteration at the α-position to the amine could attenuate VAP-1 oxidation, but here we present an effective strategy by way of generating a secondary amine that almost completely abrogates VAP-1 activity. While the VAP-1 results were in line with defined substrate requirements, the potent nature of 16e was unexpected. Based on our previous structures of RPE65, we would have predicted that the additional methyl group of 16e would interfere with critical interactions with the iron center. Another important observation described here is the RPE65 inhibition results seen with compounds 6a and 6b. When considering previous inhibition data of MB-002, we predicted a level of flexibility at the position a to the aryl moiety. Given that 6a and 6b showed no inhibition in vitro, we can now define this position as critical to the recognition element. It is likely that 6a and 6b are protonated at physiological pH values and enter the RPE65 active site in an ionized form. Assuming this is the case, we speculate that the positively charged amine group is poorly stabilized in the γ and β positions relative to the α position, which is predicted to reside in a region of high electronegativity arising from palmitate and Glu148 carboxylate moieties. This lack of electrostatic complementarity could explain the poor RPE65 inhibitory activity of 6a and 6b. Interestingly, a recent study demonstrated that sulfoximine substitution for the alcohol group of emixustat also yields a potent inhibitor, indicating that other neutral hydrogen-bond donors are tolerated at this position.

In light of the analysis above, methylated derivatives of γ-amino-α aryl alcohol chain generally matched with prediction. The α-methyl derivatives 11c and 16b displayed the lowest level of inhibition, likely due to the loss of the Thr147 hydrogen bond. Integration of a β-methyl group afforded promising compound 16c. Had this lead molecule displayed more promising in vivo properties it would have represented a new direction in the development of RPE65 inhibitors, specifically, molecules that mimic the C20 retinoid methyl group. Moving forward, the clear vertical progress we report here is 2-fold. First, we have shown how secondary amines can be accommodated into the RPE65 binding pocket and afford a new family of inhibitors that are metabolically stable to VAP-1. Second, we defined the importance of the α-aryl hydrogen-bond acceptor in RPE65 inhibition.

Most of the molecules in this example were synthesized using novel routes. Facile functional group manipulation of commercially available tyrosine derivatives led ultimately to the γ-aryl propanols 6a-c. This subtle protection strategy bypasses the potentially problematic selective installation of amino moiety to the γ or β-position of the propanol chain. The syntheses of 11d and 16b-d demonstrate the generation of the primary amine moiety by the simultaneous reduction of unsaturated nitro/cyano molecules. This strategy is comparable in length to the known aldol and Mannich-type routes employed to synthesize derivatives of emixustat. We believe the synthetic strategies displayed in this work are efficient, yet high yielding, and can be powerful general approaches for medicinal chemists interested in primary-amine-containing drug scaffolds.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A compound of formula (I): 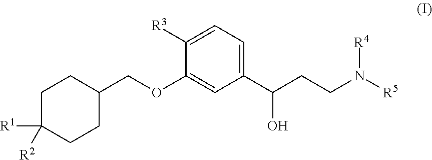
or a pharmaceutically acceptable salt, tautomer, or solvate thereof, wherein:
R¹, R², and R³ are each independently H or F;
R⁴ and R⁵ are each independently H, D, or an alkyl, which is optionally substituted with one or more D or F, and wherein at least one of R⁴ or R⁵ is an alkyl, which is optionally substituted with one or more D or F.

2. The compound of claim 1, wherein at least one of R¹, R², or R³ is F or at least one of R⁴ or R⁵ is an alkyl substituted with one or more D or F.

3. The compound of claim 1, wherein at least one of R¹ or R² is F.

4. The compound of claim 1, wherein both of R¹ and R² are F.

5. The compound of claim 1, wherein R³ is F.

6. The compound of claim 1, wherein at least one R⁴ or R⁵ is an alkyl substituted with one or more D or F.

7. The compound of claim 1, wherein at least one R⁴ or R⁵ is C₁-C₆ alkyl substituted with one or more D or F.

8. The compound of claim 7, wherein both of R⁴ and R⁵ are C₁-C₆ alkyl substituted with one or more D or F.

9. The compound of claim 7, wherein at least one R⁴ or R⁵ is methyl substituted with one or more D or F.

10. The compound of claim 7, wherein both of R⁴ and R⁵ are methyl substituted with one or more D or F.

11. The compound of claim 7, wherein at least one R⁴ or R⁵ is —CD₃ or —CF₃.

12. The compound of claim 7, wherein both of R⁴ and R⁵ are —CD₃ or —CF₃.

13. A compound selected from: 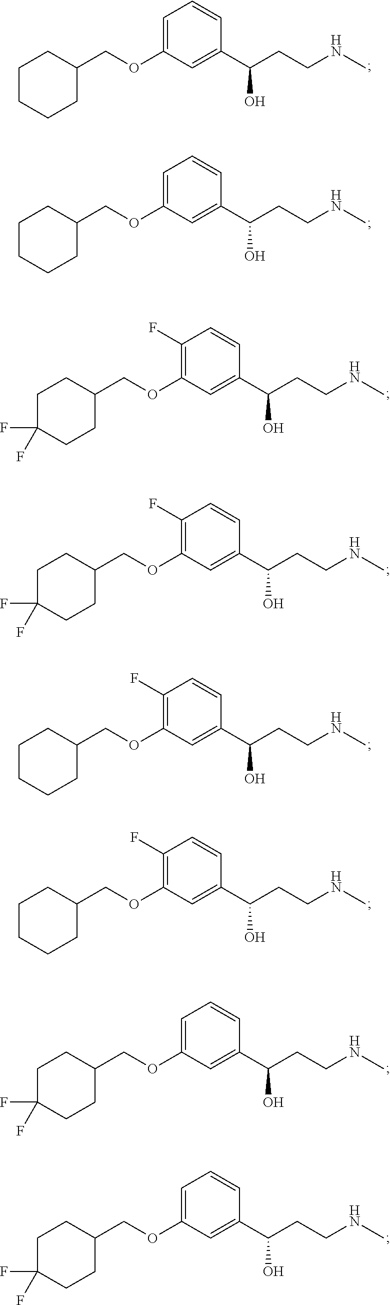 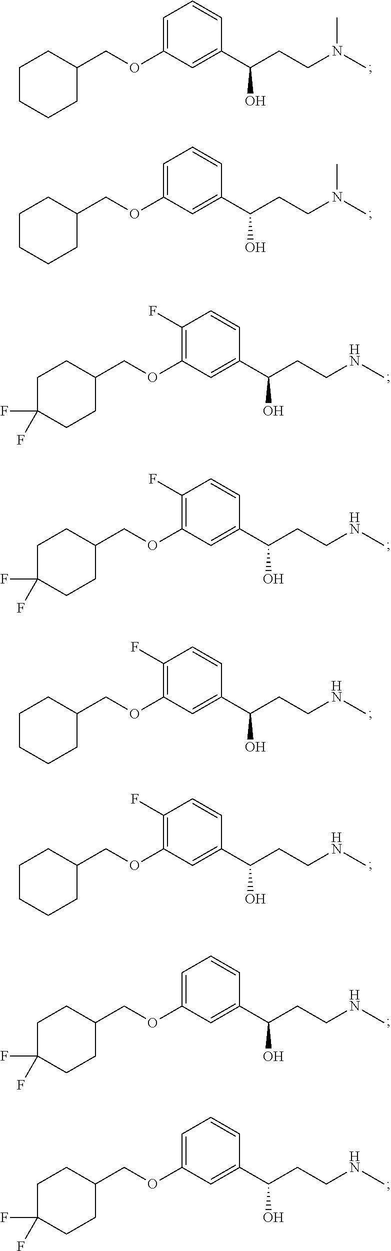 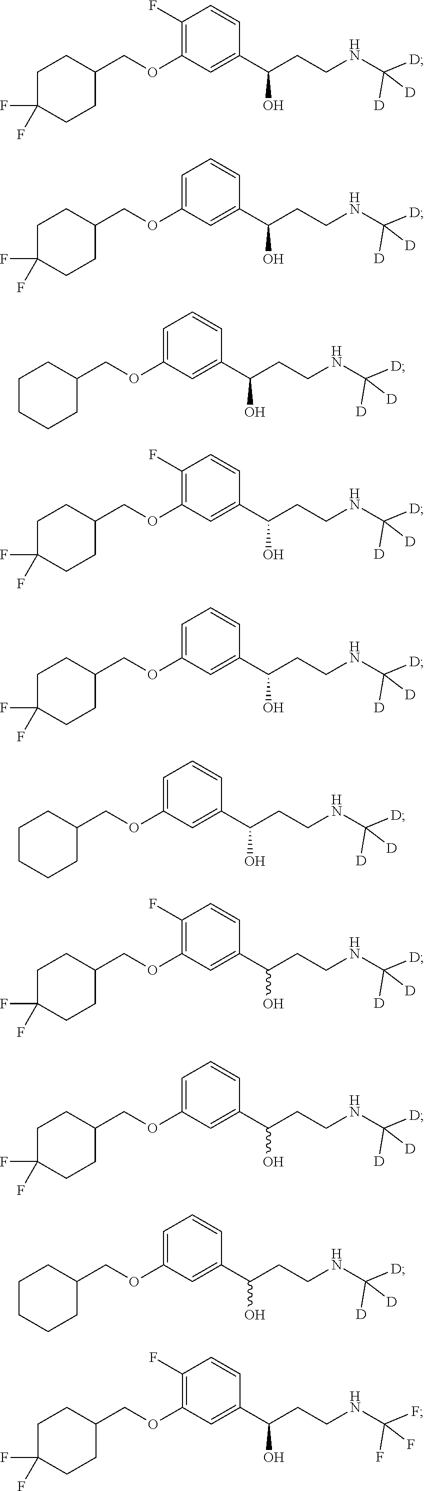 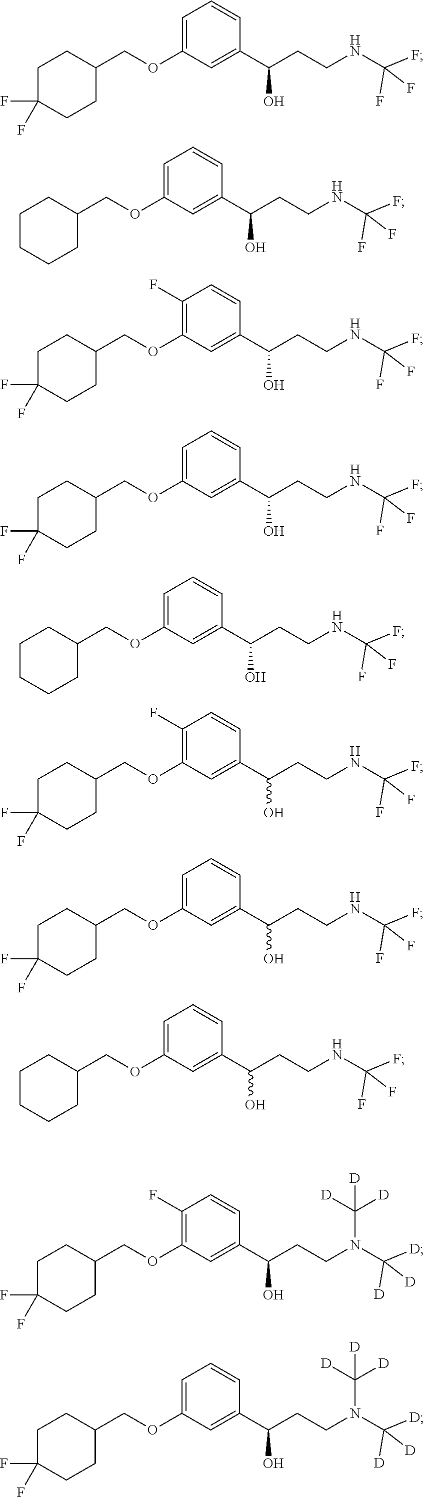 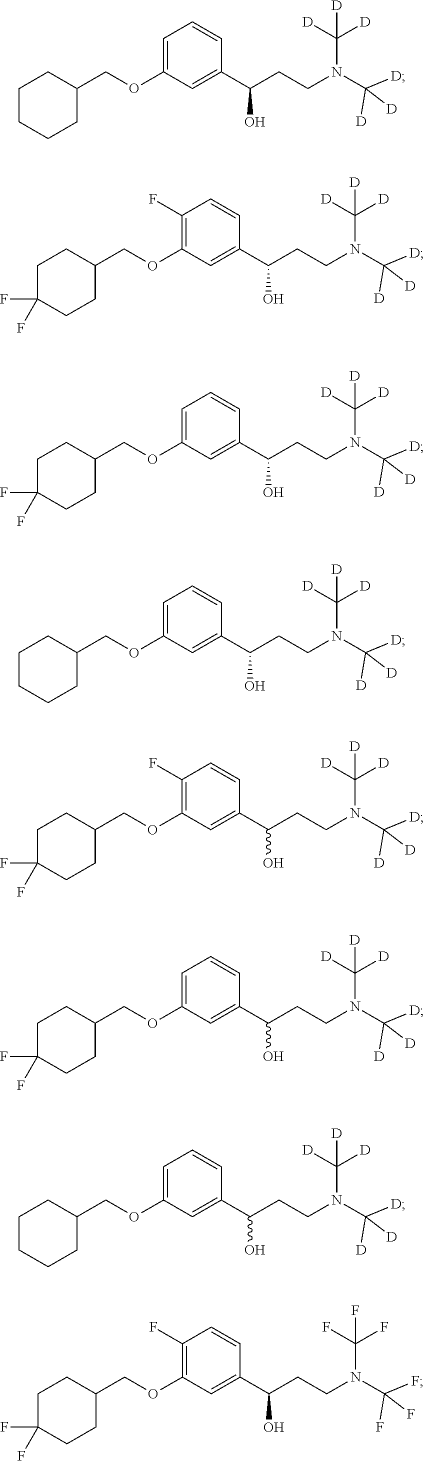 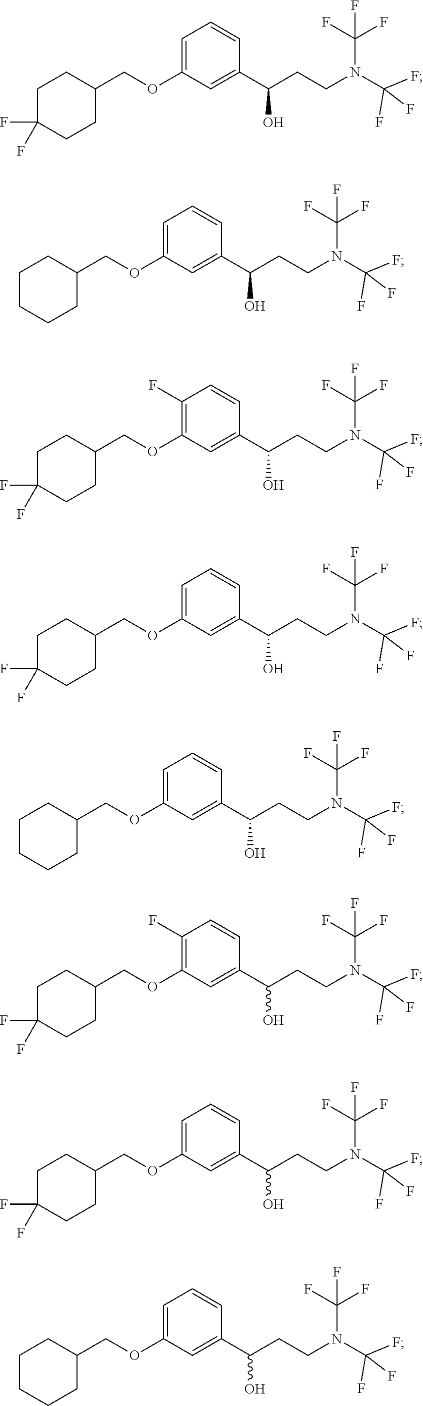
or a pharmaceutically acceptable salt, tautomer, or solvate thereof.

14. A pharmaceutical composition comprising a compound of claim 13, and a pharmaceutically acceptable carrier.

15. A method of treating an ocular disorder in a subject in need thereof, the method:
administering to the subject a therapeutically effective amount of a compound of claim 13.

16. The method of claim 15, wherein the pharmacokinetics of the compound upon administration to the subject is such that the compound does not promote night blindness when administered daily.

17. The method of claim 15, wherein the compound is not deaminated by VAP-1 enzyme.

18. The method of claim 15, the compound inhibiting RPE65 enzymatic activity in the subject.

19. The method of claim 15, the compound being administered by enteral administration.

20. The method of claim 15, the ocular disorder comprising at least one of light induced retinal degeneration, macular degeneration, Stargardt's disease, geographic atrophy, and retinitis pigmentosa.
